(19) United States
(12) Patent Application Publication
(10) Pub. No.: US 2026/0146062 A1
(43) Pub. Date: May 28, 2026

(54) COMPOUNDS AND METHODS FOR TREATING DISEASES CAUSED BY VIRUSES AND BACTERIA

(71) Applicant: The Trustees of Indiana University, Bloomington, IN (US)

(72) Inventors: William Leonard Scott, Indianapolis, IN (US); Jack Geno Samaritoni, Avon, IN (US); Martin James O'Donnell, Indianapolis, IN (US)

(21) Appl. No.: 19/122,820

(22) PCT Filed: Oct. 20, 2023
(86) PCT No.: PCT/US2023/077392

§ 371 Date: Apr. 21, 2025

Related U.S. Application Data
(60) Provisional application No. 63/380,418, filed on Oct. 21, 2022.
(60) Provisional application No. 63/491,001, filed on Mar. 17, 2023.

(51) Int. Cl. C07K 5/093; A61K 38/00; A61P 31/14; C07K 5/083

(52) U.S. Cl. CPC C07K 5/0819; A61P 31/14; C07K 5/0806; A61K 38/00

(57) ABSTRACT

Compositions and methods are provided for inhibiting the replication of severe acute respiratory syndrome coronavirus 2 (SARS-CoV-2).

### CROSS-REFERENCE TO RELATED APPLICATIONS

This application claims priority to U.S. Provisional Application Ser. No. 63/380,418, filed Oct. 21, 2022, and U.S. Provisional Application Ser. No. 63/491,001, filed Mar. 17, 2023, the entire contents of each are incorporated herein by reference.

### BACKGROUND

The severe acute respiratory syndrome coronavirus 2 (SARS-CoV-2) is a positive-sense single-stranded RNA virus that is contagious in humans and is the cause of the coronavirus disease 2019 (COVID-19) that affects all populations, worldwide, including adults and children with normal or compromised immune systems. While often asymptomatic in young healthy individuals, SARS-CoV-2 can become life-threatening in the elderly or immunocompromised individuals.

SARS-CoV-2 is a member of the subgenus Sarbecovirus (beta-CoV lineage B). SARS-CoV-2 is unique among known beta-coronaviruses in its incorporation of a polybasic cleavage site, a characteristic known to increase pathogenicity and transmissibility in other viruses.

One strategy for treating SARS-CoV-2 infections is to identify inhibitors of the main protease (Mpro) of severe acute respiratory syndrome Coronavirus-2 (SARS-CoV-2). For many viruses, protease enzymes plays a critical role in viral protein maturation by cleaving proproteins after their translation into the host cell cytosol. As a result, viral proteases are often potential drug targets. The inhibition of viral protease can reduce the assembly of mature viral particles. To date, many antiviral drugs have been developed against viral infections via targeting proteases. For instance, HIV-1 protease inhibitors and hepatitis C virus (HCV) NS3/4A protease inhibitors are amongst the FDA approved drugs.

The SARS-CoV-2 genome comprises about 30,000 nucleotides: the replicase gene of SARS-CoV-2 encodes two overlapping polyproteins—pp1a and pp1ab—that are required for viral replication and transcription. The functional polypeptides are released from the polyproteins by extensive proteolytic processing, predominantly by the 33.8-kDa M^{pro} (also known as 3C-like protease). M^{pro} digests the polyprotein at 11 conserved sites, but there may be more. The functional importance of M^{pro} in the viral life cycle, combined with the absence of closely related homologues in humans, identify M^{pro} as an attractive target for the design of antiviral drugs Therefore, formulating antiviral drugs inhibiting SARS-CoV-2 M^{pro} are anticipated to have potential clinical use.

Although promising results (such as Nirmatrelvir) have been achieved in searching for drugs inhibiting the M^{pro}, work remains to be done on designing structure-based improved drugs that inhibit M^{pro} but have improved absorption and/or distribution, metabolism, excretion and toxicological properties. The present disclosure is directed to small molecule inhibitors of M^{pro}.

### SUMMARY OF THE INVENTION

The present invention relates to the identification of antiviral compounds and antiviral compositions that exhibit potent antiviral activity against SARS-CoV-2 and have a good safety profile and thus provide an opportunity for a broad clinical use. More particularly, compounds having the general structure of Formula I

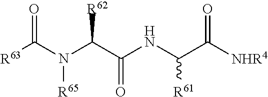

### or Formula II

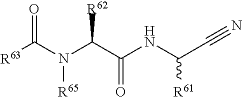

wherein
R⁶¹ is C₁-C₆ alkyl, (C₁-C₄ alkyl)R²⁵,

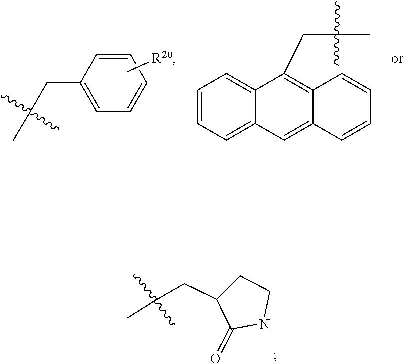

R⁶² is selected from the group consisting of C₁-C₅ alkyl,

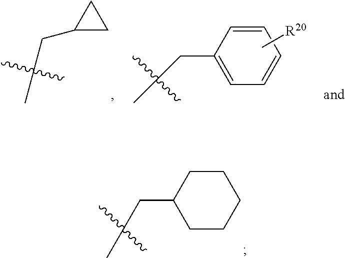

R⁶³ is selected from the group consisting of

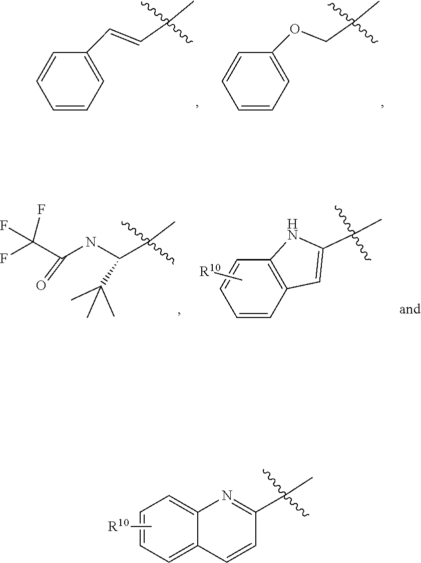

R⁴ is selected from the group consisting of H, C₁-C₄ alkyl, (C₀-C₂ alkyl)cyclopropyl, and c-propylmethyl;
R⁶⁵ is H, or R⁶² and R⁶⁵ together with the atoms to which they are attached form an optionally substituted 5 or 6 membered heterocyclic ring or a 7 membered bicyclic ring, wherein the substituents of the optionally substituted 5 or 6 membered heterocyclic ring are halo, optionally fluorine, optionally wherein the 7 membered bicyclic ring is norbornane;
R¹⁰ is H, halo or —O(C₁-C₄ alkyl);
R²⁰ is halo, optionally F, or H; and
R²⁵ is halo, CN, CONHR, NHR, COOR, phenyl sulfone, or fluorophenyl, wherein R is H or C₁-C₄ alkyl.

In one embodiment an Mpro inhibitor is provided having the general structure of

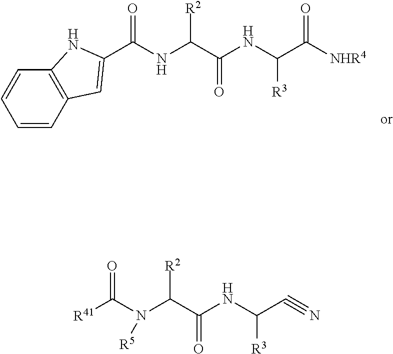

wherein
   R⁴¹ is selected from the group consisting of

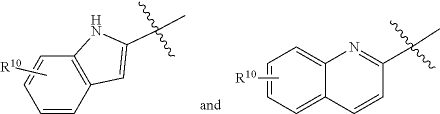

R² is selected from the group consisting of C₁-C₅ alkyl,

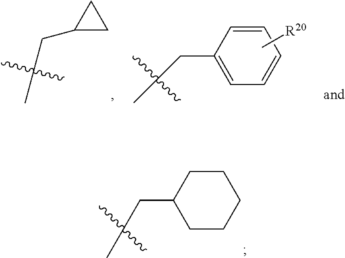

R³ is C₁-C₆ alkyl, (C₁-C₄ alkyl)R²⁵,

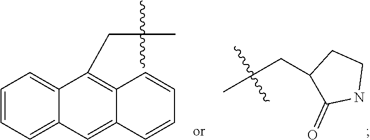 [SMILES: *Cc1c2ccccc2cc2ccccc12 ; *CC1CCNC1=O]

R⁴ is selected from the group consisting of H, C₁-C₄ alkyl, (C₀-C₂ alkyl)cyclopropyl, and c-propylmethyl;
R⁵ is H, or R² and R⁵ together with the atoms to which they are attached form an optionally substituted 5 or 6 membered heterocyclic ring or a 7 membered bicyclic ring, wherein the substituents of the optionally substituted 5 or 6 membered heterocyclic ring are halo, optionally fluorine, optionally wherein the 7 membered bicyclic ring is norbornane;
R¹⁰ is H, halo or —O(C₁-C₄ alkyl);
R²⁰ is halo, optionally F, or H; and
R²¹ is halo, CN, CONHR, NHR, COOR, phenyl sulfone, or fluorophenyl, wherein R is H or C₁-C₄ alkyl. In a further embodiment the compound has the general structure of

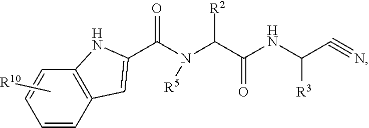

wherein
R¹⁰ is H or methoxy;
R² is selected from the group consisting of C₁-C₅ alkyl,

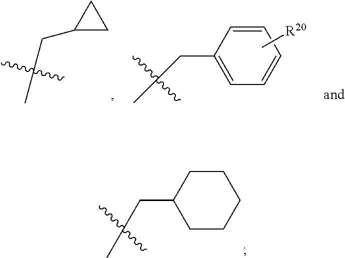

R³ is C₁-C₆ alkyl, (C₁-C₄ alkyl)R²⁵,

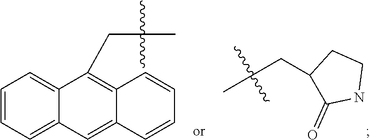 [SMILES: *Cc1c2ccccc2cc2ccccc12 ; *CC1CCNC1=O]

R⁵ is H, or R² and R⁵ together with the atoms to which they are attached form an optionally substituted 5 or 6 membered heterocyclic ring or a 7 membered bicyclic ring, wherein the substituents of the optionally substituted 5 or 6 membered heterocyclic ring are halo, optionally fluorine, optionally wherein the 7 membered bicyclic ring is norbornane; and
R²⁵ is halo, CN, CONHR, NHR, COOR, phenyl sulfone, or fluorophenyl, wherein R is H or C₁-C₄ alkyl.

In one embodiment the compound has the general structure of

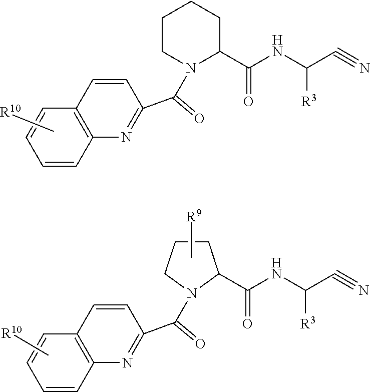

wherein
R³ is C₁-C₆ alkyl, (C₁-C₄ alkyl)R²⁵,

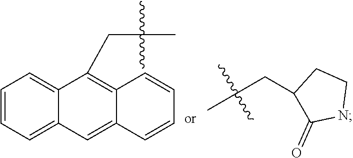

R⁹ is H or F;
R¹⁰ is H or methoxy; and
R²⁵ is halo, CN, CONHR, NHR, COOR, phenyl sulfone, or fluorophenyl, wherein R is H or C₁-C₄ alkyl.

In one embodiment the compound has the general structure of

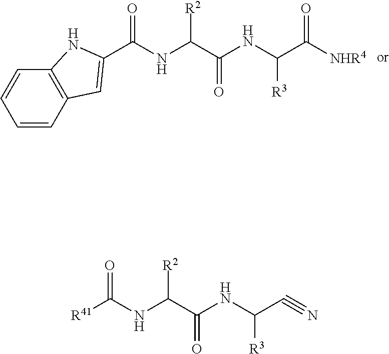

wherein
   R² is selected from the group consisting of C₁-C₄ alkyl,

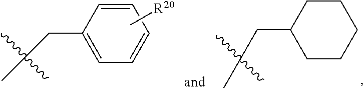

R³ is C₁-C₆ alkyl, (C₁-C₄ alkyl)R²⁵,

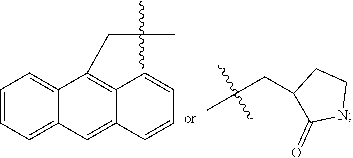

R⁴ is selected from the group consisting of H, C₁-C₄ alkyl, (C₀-C₂ alkyl)cyclopropyl, and c-propylmethyl;
R²⁰ is halo, optionally F, or H; and
R²⁵ is halo, CN, CONHR, NHR. COOR, phenyl sulfone, or fluorophenyl, wherein R is H or C₁-C₄ alkyl are provided, with the proviso that the compound is not

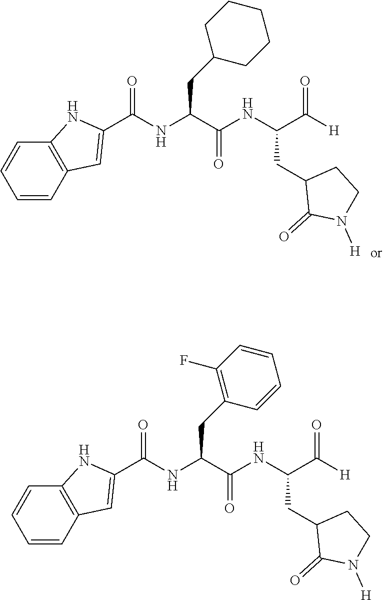 [SMILES: O=C[C@H](CC1CCNC1=O)NC(=O)[C@H](CC1CCCCC1)NC(=O)c1cc2ccccc2[nH]1 ; O=C[C@H](CC1CCNC1=O)NC(=O)[C@H](Cc1ccccc1F)NC(=O)c1cc2ccccc2[nH]1]

and wherein the compounds have activity in inhibiting the activity of M^{pro}. In accordance with one embodiment any of the compounds shown in the Representative Embodiments can be used to inhibit the activity of M^{pro} and prevent or treat a SARS-CoV-2 infection. The invention also provides pharmaceutical compositions comprising such antiviral compounds or antiviral compositions as well as methods to use the antiviral compounds, antiviral compositions and pharmaceutical compositions to inhibit, SARS-CoV-2 replication or reactivation, and to treat disease conditions associated with or caused by SARS-CoV-2. Further objects of this invention are described herein below.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein

### BRIEF DESCRIPTION OF THE DRAWINGS

FIG. 1 shows a synthesis scheme for forming the inhibitors having an amide.

FIG. 2 shows a synthesis scheme for converting the amide to a nitrile.

FIG. 3 shows an exemplary synthetic scheme.

FIG. 4 is a graph showing the inhibition ability of Mpro for several disclosed inhibitors.

FIG. 5 is a graph showing the inhibitors reduce the plaque formation of cells infected with a SARS-CoV2 virus.

FIG. 6 is a graph showing the increased inhibition ability of Compound No. 112 with increased concentration.

FIG. 7 is a graph showing the ability of several inhibitors at 2 micromolar.

FIG. 8 is a graph showing the ability of several inhibitors at 2 micromolar.

FIG. 9 shows graphs comparing inhibition to cell toxicity.

FIG. 10 shows graphs comparing inhibition to cell toxicity.

FIG. 11 shows graphs comparing inhibition to cell toxicity.

### DETAILED DESCRIPTION

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present disclosure can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted. As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C1-30 for straight chains, C3-30 for branched chains), and more preferably 20 or fewer. Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. Coalkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

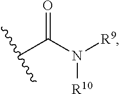

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

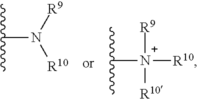

wherein R⁹, R¹⁰, and R¹⁰, each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

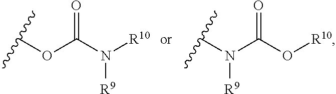

wherein R⁹ and R¹¹ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo [2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbonate” is art-recognized and refers to a group —OCO₂—.

The term “carboxy”, as used herein, refers to a group represented by the formula CO₂H.

The term “ester”, as used herein, refers to a group —C(O)OR⁸, wherein R⁸ represents a hydrocarbyl group.

The term “ketone”, as used herein, refers to a group —C(O)R⁷, wherein R⁷ represents a hydrocarbyl group (e.g., alkyl, aryl, heteroaryl).

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

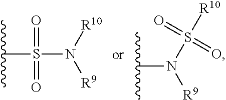

wherein R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—R⁹, wherein R⁹ represents hydrocarbyl (e.g., alkyl, aryl, heteroaryl).

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁸ or —SC(O)R⁸ wherein R⁸ represents a hydrocarbyl.

The term “thioether”, as used herein, is equivalent to an ether, wherein the oxygen is replaced with a sulfur.

The term “urea” is art-recognized and may be represented by the general formula

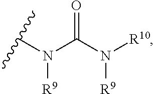

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients. The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

As used herein, the term “Mpro inhibitor” as used herein refers to a compound that prevents, reverses, slows, or inhibits the activity of the viral main protease (Mpro).

In treatment methods according to the disclosure, an “effective amount” means an amount or dose sufficient to generally bring about the desired therapeutic benefit in subjects needing such treatment. Effective amounts or doses of the compounds of the disclosure may be ascertained by routine methods, such as modeling, dose escalation, or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about from about 0.1 mg to 1 g daily, or about 1 mg to 50 mg daily, or about 50 to 250 mg daily, or about 250 mg to 1 g daily. The total dosage may be given in single or divided dosage units (e.g., BID, TID, QID).

### Embodiments

In accordance with the present disclosure compositions and methods are provided for inhibiting the replication of coronaviruses including severe acute respiratory syndrome coronavirus 2 (SARS-CoV-2). More particularly, inhibitor are provided that bind to the SARS-CoV-2 M^{pro} protease. In one embodiment an inhibitor of M^{pro} (also referred to herein as Mpro) is provided wherein the inhibitor has the general structure of:
compounds having the general structure of Formula (I)

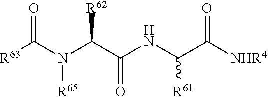

### or Formula (II)

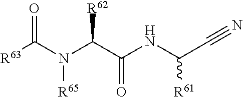

wherein
   R⁶¹ is C₁-C₆ alkyl, (C₁-C₄ alkyl)R²⁵,

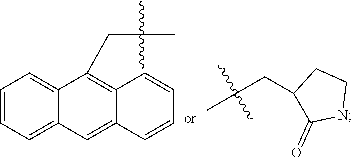

R⁶² is selected from the group consisting of C₁-C₅ alkyl,

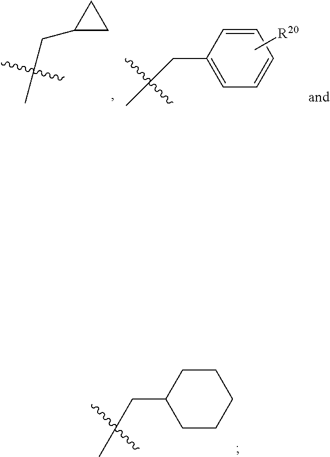

R⁶³ is selected from the group consisting of

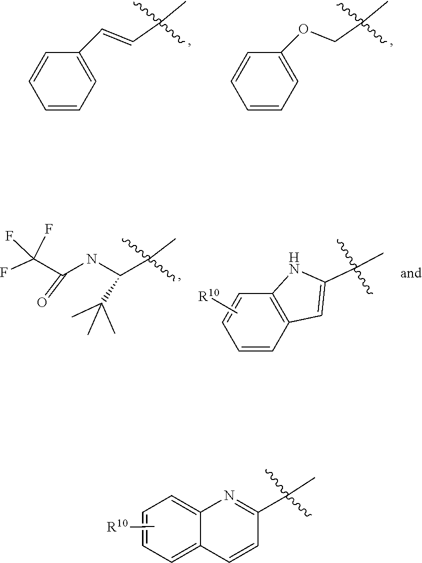

R⁴ is selected from the group consisting of H, C₁-C₄ alkyl, (C₀-C₂ alkyl)cyclopropyl, and c-propylmethyl;
R⁶⁵ is H, or R⁶² and R⁶⁵ together with the atoms to which they are attached form an optionally substituted 5 or 6 membered heterocyclic ring or a 7 membered bicyclic ring, wherein the substituents of the optionally substituted 5 or 6 membered heterocyclic ring are halo, optionally fluorine, optionally wherein the 7 membered bicyclic ring is norbornane;
R¹⁰ is H, halo or —O(C₁-C₄ alkyl);
R²⁰ is halo, optionally F, or H; and
R²⁵ is halo, CN, CONHR, NHR, COOR, phenyl sulfone, or fluorophenyl, wherein R is H or C₁-C₄ alkyl.

In one embodiment a Mpro inhibitor is provided having the general structure of

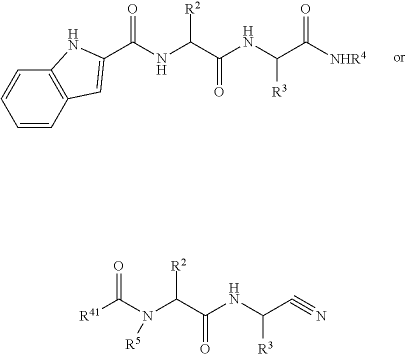

wherein
   R⁴¹ is selected from the group consisting of

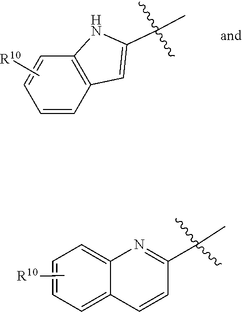

R² is selected from the group consisting of C₁-C₅ alkyl,

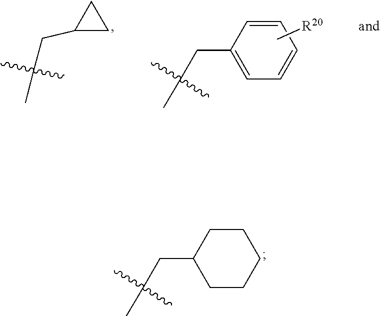

R³ is C₁-C₆ alkyl, (C₁-C₄ alkyl)R²⁵,

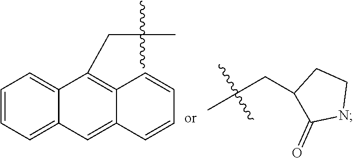

R⁴ is selected from the group consisting of H, C₁-C₄ alkyl, (C₀-C₂ alkyl)cyclopropyl, and c-propylmethyl;
R⁵ is H, or R² and R⁵ together with the atoms to which they are attached form an optionally substituted 5 or 6 membered heterocyclic ring or a 7 membered bicyclic ring, wherein the substituents of the optionally substituted 5 or 6 membered heterocyclic ring are halo, optionally fluorine, optionally wherein the 7 membered bicyclic ring is norbornane;
R¹⁰ is H, halo or —O(C₁-C₄ alkyl);
R²⁰ is halo, optionally F, or H; and
R²⁵ is halo, CN, CONHR, NHR, COOR, phenyl sulfone, or fluorophenyl, wherein R is H or C₁-C₄ alkyl. In a further embodiment the compound has the general structure of

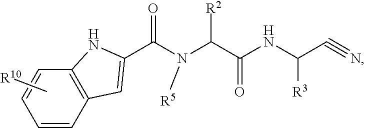

R, wherein
R¹⁰ is H or methoxy;
R² is selected from the group consisting of C₁-C₅ alkyl,

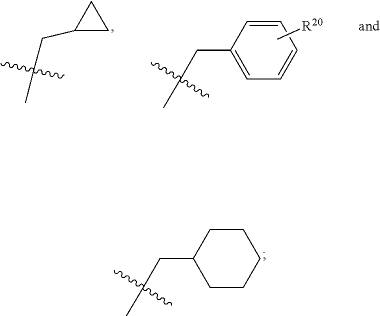

R³ is C₁-C₆ alkyl, (C₁-C₄ alkyl)R²⁵,

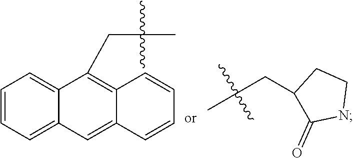

R⁵ is H, or R² and R⁵ together with the atoms to which they are attached form an optionally substituted 5 or 6 membered heterocyclic ring or a 7 membered bicyclic ring, wherein the substituents of the optionally substituted 5 or 6 membered heterocyclic ring are halo, optionally fluorine, optionally wherein the 7 membered bicyclic ring is norbornane; and
R²¹ is halo, CN, CONHR, NHR, COOR, phenyl sulfone, or fluorophenyl, wherein R is H or C₁-C₄ alkyl.

In one embodiment the compound has the general structure of

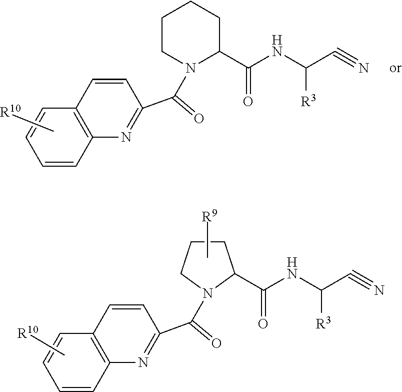

wherein
R³ is C₁-C₆ alkyl, (C₁-C₄ alkyl)R²⁵,

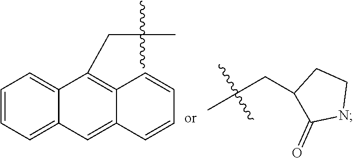

R⁹ is H or F;
R¹⁰ is H or methoxy; and
R²¹ is halo, CN, CONHR, NHR, COOR, phenyl sulfone, or fluorophenyl, wherein R is H or C₁-C₄ alkyl.

In one embodiment the compound has the general structure of

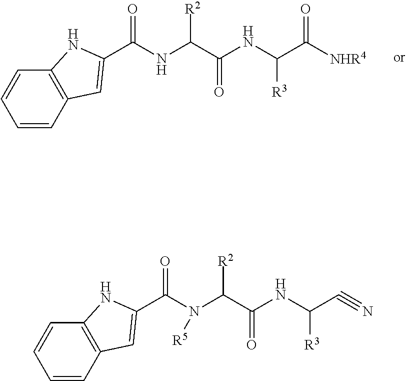

wherein
   R² is selected from the group consisting of C₁-C₅ alkyl,

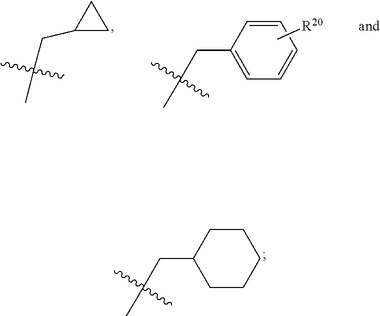

R³ is C₁-C₆ alkyl, (C₁-C₄ alkyl)R²⁵,

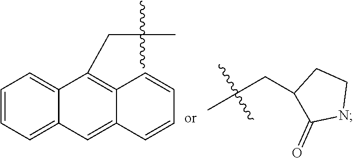

R⁴ is selected from the group consisting of H, C₁-C₄ alkyl, (C₀-C₂ alkyl)cyclopropyl, and c-propylmethyl;
R⁵ is H, or R² and R⁵ together with the atoms to which they are attached form an optionally substituted 5 or 6 membered heterocyclic ring or a 7 membered bicyclic ring, wherein the substituents of the optionally substituted 5 or 6 membered heterocyclic ring are halo, optionally fluorine, optionally wherein the 7 membered bicyclic ring is norbornane;
R²⁰ is halo, optionally F, or H; and
R²⁵ is halo, CN, CONHR, NHR, COOR, phenyl sulfone, or fluorophenyl, wherein R is H or C₁-C₄ alkyl. In one embodiment a compound of Formula I is provided wherein R² is isobutyl. In one embodiment a compound of Formula I is provided wherein
R³ is

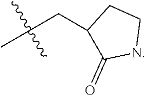

In one embodiment one or more compounds as disclosed in the Representative Embodiments is used to inhibit M^{pro} activity and prevent or treat a SARS-CoV-2 infection.

In one embodiment an inhibitor of M^{pro} is provided wherein the inhibitor has the general structure of:

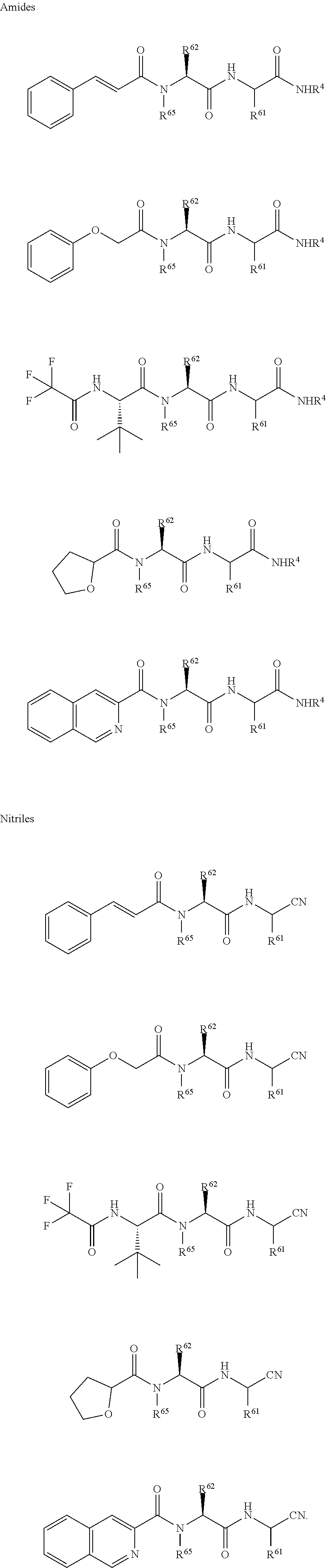

wherein
R⁶¹ is C₁-C₆ alkyl, (C₁-C₄ alkyl)R²⁵,

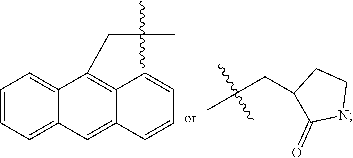

R⁶² is selected from the group consisting of C₁-C₅ alkyl,

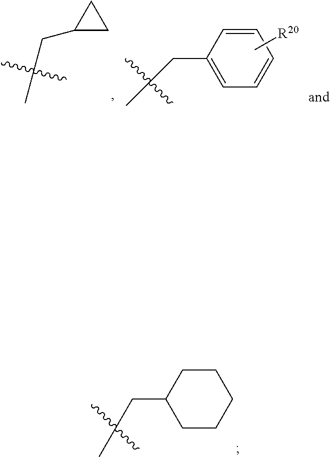

R⁴ is selected from the group consisting of H, C₁-C₄ alkyl, (C₀-C₂ alkyl)cyclopropyl, and c-propylmethyl;
R⁶⁵ is H, or R⁶² and R⁶⁵ together with the atoms to which they are attached form an optionally substituted 5 or 6 membered heterocyclic ring or a 7 membered bicyclic ring, wherein the substituents of the optionally substituted 5 or 6 membered heterocyclic ring are halo, optionally fluorine, optionally wherein the 7 membered bicyclic ring is norbornane;
R¹⁰ is H, halo or —O(C₁-C₄ alkyl);
R²⁰ is halo, optionally F, or H; and
R²⁵ is halo, CN, CONHR, NHR, COOR, phenyl sulfone, or fluorophenyl, wherein R is H or C₁-C₄ alkyl.

In one embodiment an inhibitor of M^{pro} is provided wherein the inhibitor has the general structure of:

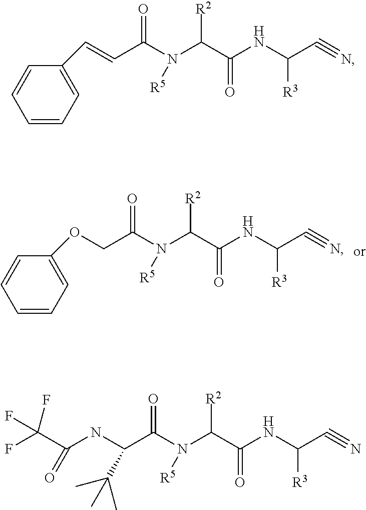

wherein
   R² is selected from the group consisting of C₁-C₅ alkyl;
   R³ is

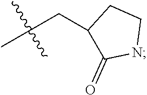

R⁵ is H, or R² and R⁵ together with the atoms to which they are attached form an optionally substituted 5 or 6 membered heterocyclic ring or a 7 membered bicyclic ring, wherein the substituents of the optionally substituted 5 or 6 membered heterocyclic ring are halo, optionally fluorine, optionally wherein the 7 membered bicyclic ring is norbornane.

In one embodiment an inhibitor of M^{pro} is provided wherein the inhibitor has the general structure of:

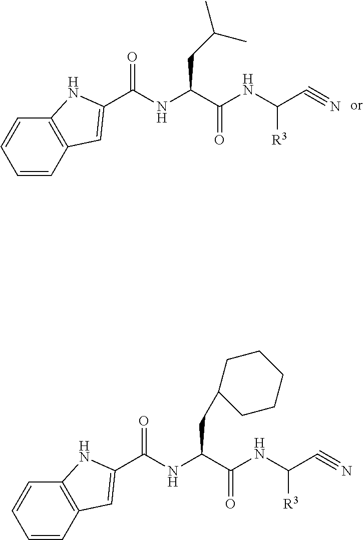

wherein
   R³ is C₁-C₆ alkyl, (C₁-C₅ alkyl)R²⁵,

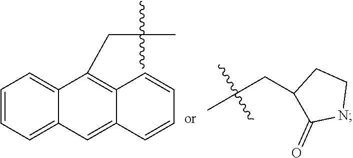

R²⁵ is halo, CN, CONHR, NHR, COOR, phenyl sulfone, or fluorophenyl, wherein R is H or C₁-C₄ alkyl.

In one embodiment an M^{pro} inhibitor is provided wherein the compound has the structure of

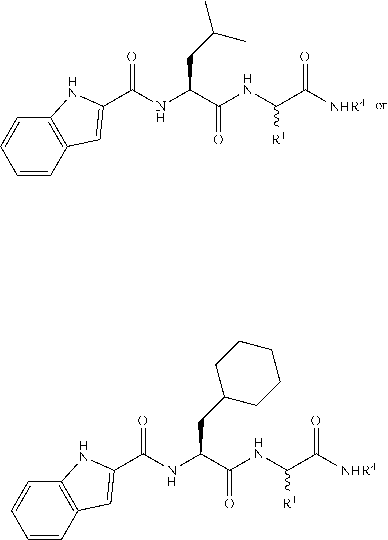

wherein R¹ is C₁-C₆ alkyl, (C₁-C₄ alkyl)R²⁵

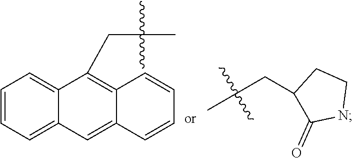

R⁴ is selected from the group consisting of H, C₁-C₄ alkyl; and
R²⁵ is halo, CN, CONHR, NHR. COOR, phenyl sulfone, or fluorophenyl, wherein R is H or C₁-C₄ alkyl.

In one embodiment an Mpro inhibitor is provided wherein the compound has the structure of

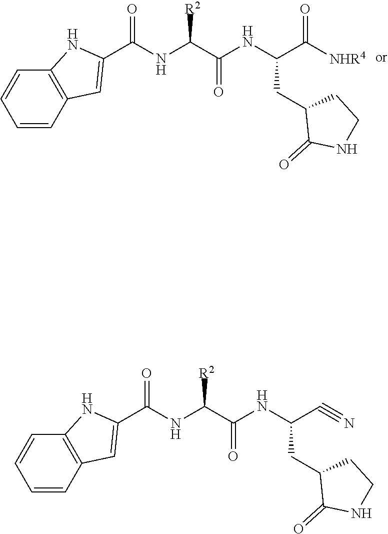

wherein R² is C₁-C₄ alkyl or

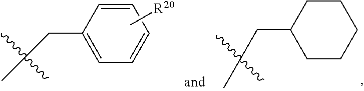

wherein R²⁰ is Fluoro, optionally wherein R² is Me, i-Bu, 3FBn, n-Bu, 4FBn, or c-C₆H₁₁CH₂; and
   R⁴ is H or C₁-C₄ alkyl, optionally wherein R⁴ is Me, n-propyl, i-Bu, c-propyl, or c-propylmethyl. In one embodiment one or more compounds as disclosed in the Representative Embodiments is used to inhibit Mpro activity and prevent or treat a SARS-CoV-2 infection.

In accordance with one embodiment a method of treating or preventing COVID-19 and/or a SARS-CoV-2 infection in a patient is provided by administering to the patient an antivirally effective amount of an antiviral compound or antiviral composition or pharmaceutical composition comprising any of the M^{pro} inhibitors disclosed herein either alone or in combination with at least one other antiviral agent, administered together or separately.

In accordance with one embodiment a method of inhibiting the replication of SARS-CoV-2 comprising exposing the virus to an effective amount of any of the Mpro inhibitors disclosed herein either alone or in combination with at least one other antiviral agent, administered together or separately under conditions where replication of SARS-CoV-2 is inhibited. This method can be practiced in vitro or in vivo.

One aspect of the present disclosure is directed to pharmaceutical compositions comprising a Mpro inhibitor as disclosed herein. In one embodiment the antiviral compounds or antiviral compositions of the present disclosure is co-administered with at least one additional agent selected from: a virus entry inhibitor, a virus early transcription event inhibitor, another virus RNA polymerase inhibitor such as remdesivir, a virus protease inhibitor such as lopinavir or ritonavir, a virus terminase inhibitor, a virus maturation inhibitor, an inhibitor of another target in the virus life cycle such as, hydroxychloroqione or pharmaceutically acceptable salts thereof.

These additional agents may be combined with the M^{pro} inhibitors disclosed to create a single pharmaceutical dosage form. Alternatively, these additional agents may be separately administered to the patient as part of a multiple dosage form, for example, using a kit. Such additional agents may be administered to the patient prior to, concurrently with, or following the administration of a pharmaceutical composition comprising one or more of the M^{pro} inhibitors disclosed herein.

The pharmaceutical compositions of the present disclosure can be administered by known methods, including oral, parenteral, inhalation, and the like. In certain embodiments, the Mpro inhibitors disclosed herein is administered orally, as a pill, lozenge, troche, capsule. In other embodiments, pharmaceutical compositions of the invention are administered by injection or infusion. In other embodiments, the pharmaceutical composition is administered intranasally or by inhalation.

The antiviral compounds or antiviral compositions disclosed herein may also be used in combination with other agents, including for example an additional antiviral agent other than the antiviral compounds or antiviral compositions of the invention for treatment of a viral infection in a subject. The term “combination”, as used herein is meant either a fixed combination in one dosage unit form, as separate dosage forms suitable for use together either simultaneously or sequentially, or as a kit of parts for the combined administration where a compound of the present invention and a combination partner may be administered independently at the same time or separately within time intervals that especially allow that the combination partners show a cooperative, e.g., synergistic, effect, or any combination thereof.

REPRESENTATIVE EMBODIMENTS

| Compound | |
|---|---|
| Number | Structure |
| 1 | 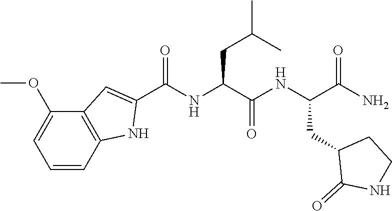 [SMILES: COc1cccc2[nH]c(C(=O)N[C@@H](CC(C)C)C(=O)N[C@@H](C[C@@H]3CCNC3=O)C(N)=O)cc12] |
| 2 | 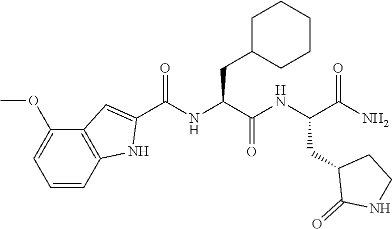 [SMILES: COc1cccc2[nH]c(C(=O)N[C@@H](CC3CCCCC3)C(=O)N[C@@H](C[C@@H]3CCNC3=O)C(N)=O)cc12] |
| 3 | 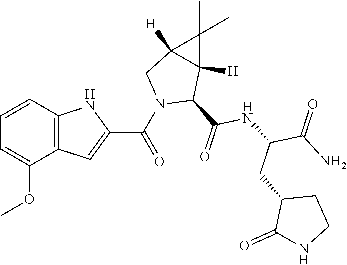 [SMILES: COc1cccc2[nH]c(C(=O)N3C[C@H]4[C@@H]([C@H]3C(=O)N[C@@H](C[C@@H]3CCNC3=O)C(N)=O)C4(C)C)cc12] |
| 4 | 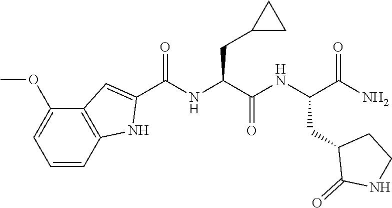 [SMILES: COc1cccc2[nH]c(C(=O)N[C@@H](CC3CC3)C(=O)N[C@@H](C[C@@H]3CCNC3=O)C(N)=O)cc12] |
| 5 | 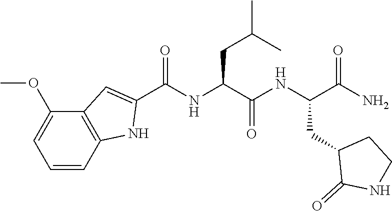 [SMILES: COc1cccc2[nH]c(C(=O)N[C@@H](CC(C)C)C(=O)N[C@@H](C[C@@H]3CCNC3=O)C(N)=O)cc12] |
| 6 | 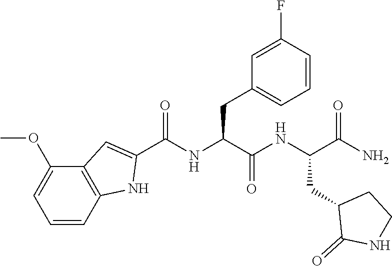 [SMILES: COc1cccc2[nH]c(C(=O)N[C@@H](Cc3cccc(F)c3)C(=O)N[C@@H](C[C@@H]3CCNC3=O)C(N)=O)cc12] |
| 7 | 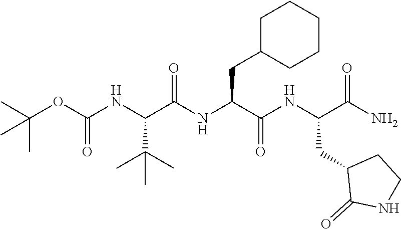 [SMILES: CC(C)(C)OC(=O)N[C@H](C(=O)N[C@@H](CC1CCCCC1)C(=O)N[C@@H](C[C@@H]1CCNC1=O)C(N)=O)C(C)(C)C] |
| 8 | 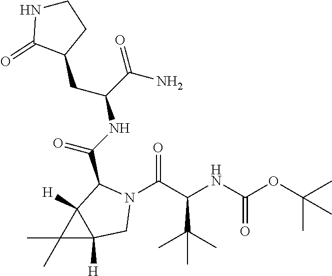 [SMILES: CC(C)(C)OC(=O)N[C@H](C(=O)N1C[C@H]2[C@@H]([C@H]1C(=O)N[C@@H](C[C@@H]1CCNC1=O)C(N)=O)C2(C)C)C(C)(C)C] |
| 9 | 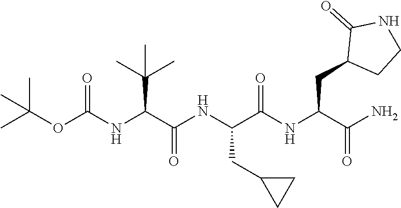 [SMILES: CC(C)(C)OC(=O)N[C@H](C(=O)N[C@@H](CC1CC1)C(=O)N[C@@H](C[C@@H]1CCNC1=O)C(N)=O)C(C)(C)C] |
| 10 | 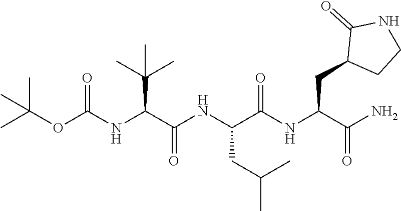 [SMILES: CC(C)C[C@H](NC(=O)[C@@H](NC(=O)OC(C)(C)C)C(C)(C)C)C(=O)N[C@@H](C[C@@H]1CCNC1=O)C(N)=O] |
| 11 | 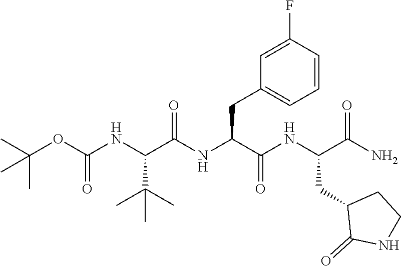 [SMILES: CC(C)(C)OC(=O)N[C@H](C(=O)N[C@@H](Cc1cccc(F)c1)C(=O)N[C@@H](C[C@@H]1CCNC1=O)C(N)=O)C(C)(C)C] |
| 12 | 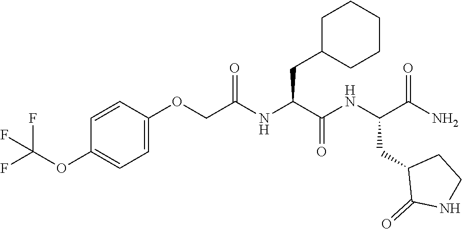 [SMILES: NC(=O)[C@H](C[C@@H]1CCNC1=O)NC(=O)[C@H](CC1CCCCC1)NC(=O)COc1ccc(OC(F)(F)F)cc1] |
| 13 | 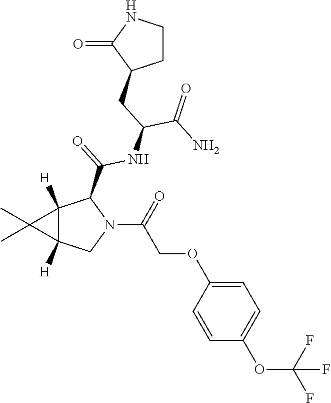 [SMILES: CC1(C)[C@@H]2[C@@H](C(=O)N[C@@H](C[C@@H]3CCNC3=O)C(N)=O)N(C(=O)COc3ccc(OC(F)(F)F)cc3)C[C@@H]21] |
| 14 | 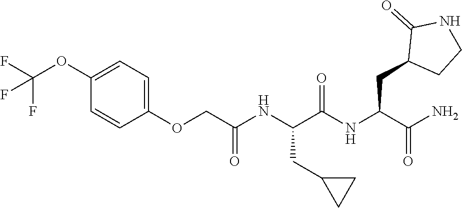 [SMILES: NC(=O)[C@H](C[C@@H]1CCNC1=O)NC(=O)[C@H](CC1CC1)NC(=O)COc1ccc(OC(F)(F)F)cc1] |
| 15 | 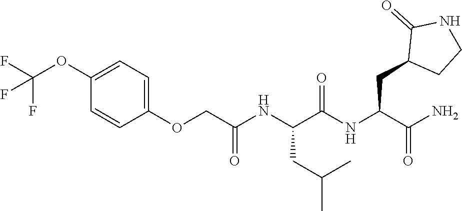 [SMILES: CC(C)C[C@H](NC(=O)COc1ccc(OC(F)(F)F)cc1)C(=O)N[C@@H](C[C@@H]1CCNC1=O)C(N)=O] |
| 16 | 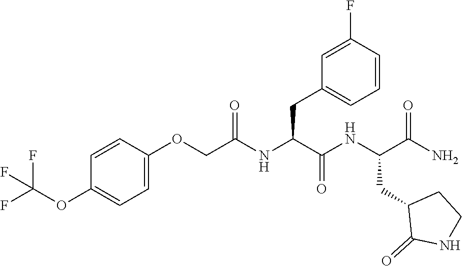 [SMILES: NC(=O)[C@H](C[C@@H]1CCNC1=O)NC(=O)[C@H](Cc1cccc(F)c1)NC(=O)COc1ccc(OC(F)(F)F)cc1] |
| 17 | 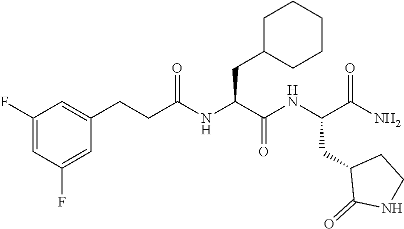 [SMILES: NC(=O)[C@H](C[C@@H]1CCNC1=O)NC(=O)[C@H](CC1CCCCC1)NC(=O)CCc1cc(F)cc(F)c1] |
| 18 | 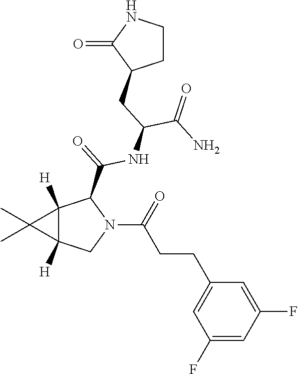 [SMILES: CC1(C)[C@@H]2[C@@H](C(=O)N[C@@H](C[C@@H]3CCNC3=O)C(N)=O)N(C(=O)CCc3cc(F)cc(F)c3)C[C@@H]21] |
| 19 | 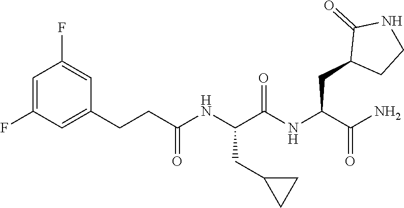 [SMILES: NC(=O)[C@H](C[C@@H]1CCNC1=O)NC(=O)[C@H](CC1CC1)NC(=O)CCc1cc(F)cc(F)c1] |
| 20 | 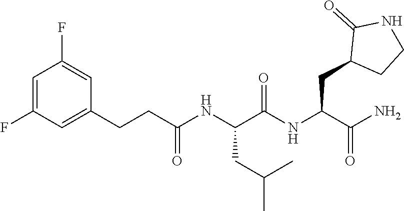 [SMILES: CC(C)C[C@H](NC(=O)CCc1cc(F)cc(F)c1)C(=O)N[C@@H](C[C@@H]1CCNC1=O)C(N)=O] |
| 21 | 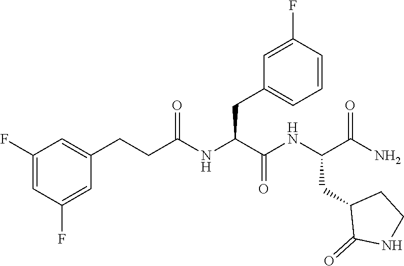 [SMILES: NC(=O)[C@H](C[C@@H]1CCNC1=O)NC(=O)[C@H](Cc1cccc(F)c1)NC(=O)CCc1cc(F)cc(F)c1] |
| 22 | 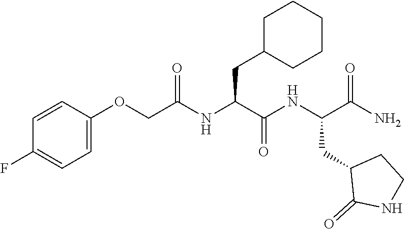 [SMILES: NC(=O)[C@H](C[C@@H]1CCNC1=O)NC(=O)[C@H](CC1CCCCC1)NC(=O)COc1ccc(F)cc1] |
| 23 | 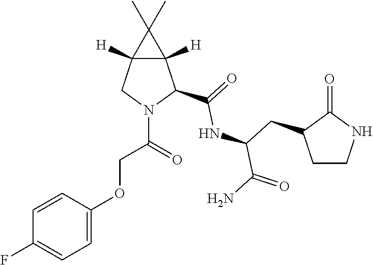 |
| 24 | 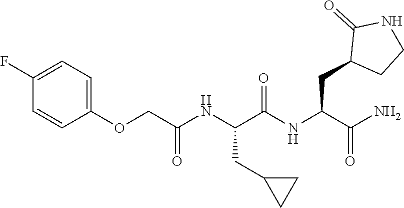 [SMILES: NC(=O)[C@H](C[C@@H]1CCNC1=O)NC(=O)[C@H](CC1CC1)NC(=O)COc1ccc(F)cc1] |
| 25 | 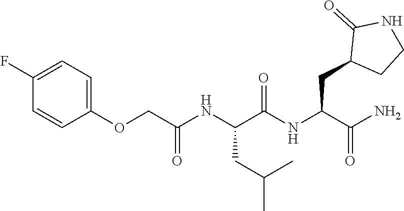 [SMILES: CC(C)C[C@H](NC(=O)COc1ccc(F)cc1)C(=O)N[C@@H](C[C@@H]1CCNC1=O)C(N)=O] |
| 26 | 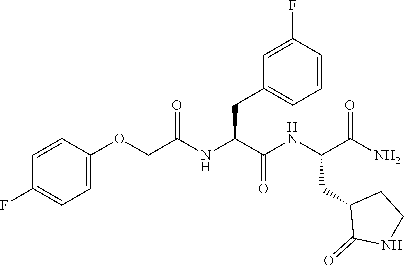 [SMILES: NC(=O)[C@H](C[C@@H]1CCNC1=O)NC(=O)[C@H](Cc1cccc(F)c1)NC(=O)COc1ccc(F)cc1] |
| 27 | 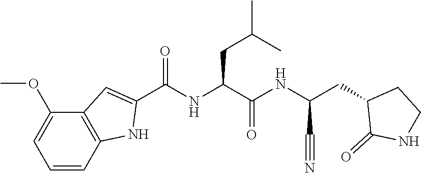 [SMILES: COc1cccc2[nH]c(C(=O)N[C@@H](CC(C)C)C(=O)N[C@H](C#N)C[C@@H]3CCNC3=O)cc12] |
| 28 | 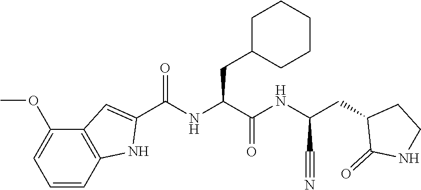 [SMILES: COc1cccc2[nH]c(C(=O)N[C@@H](CC3CCCCC3)C(=O)N[C@H](C#N)C[C@@H]3CCNC3=O)cc12] |
| 29 | 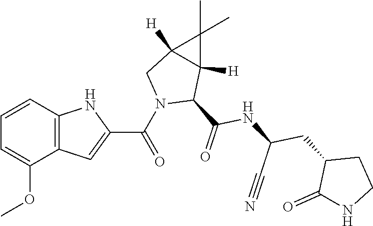 [SMILES: COc1cccc2[nH]c(C(=O)N3C[C@H]4[C@@H]([C@H]3C(=O)N[C@H](C#N)C[C@@H]3CCNC3=O)C4(C)C)cc12] |
| 30 | 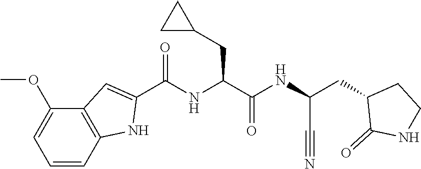 [SMILES: COc1cccc2[nH]c(C(=O)N[C@@H](CC3CC3)C(=O)N[C@H](C#N)C[C@@H]3CCNC3=O)cc12] |
| 31 | 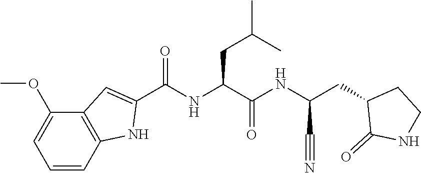 [SMILES: COc1cccc2[nH]c(C(=O)N[C@@H](CC(C)C)C(=O)N[C@H](C#N)C[C@@H]3CCNC3=O)cc12] |
| 32 | 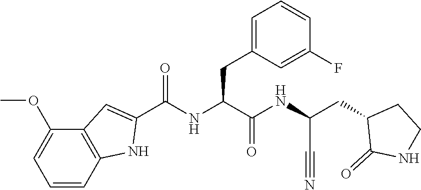 [SMILES: COc1cccc2[nH]c(C(=O)N[C@@H](Cc3cccc(F)c3)C(=O)N[C@H](C#N)C[C@@H]3CCNC3=O)cc12] |
| 33 | 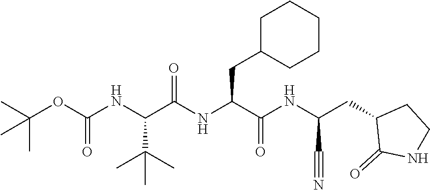 [SMILES: CC(C)(C)OC(=O)N[C@H](C(=O)N[C@@H](CC1CCCCC1)C(=O)N[C@H](C#N)C[C@@H]1CCNC1=O)C(C)(C)C] |
| 34 | 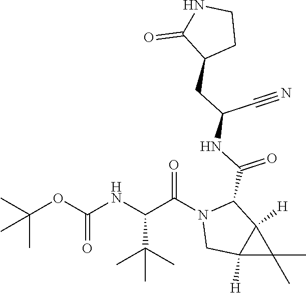 [SMILES: CC(C)(C)OC(=O)N[C@H](C(=O)N1C[C@H]2[C@@H]([C@H]1C(=O)N[C@H](C#N)C[C@@H]1CCNC1=O)C2(C)C)C(C)(C)C] |
| 35 | 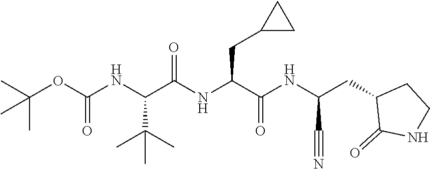 [SMILES: CC(C)(C)OC(=O)N[C@H](C(=O)N[C@@H](CC1CC1)C(=O)N[C@H](C#N)C[C@@H]1CCNC1=O)C(C)(C)C] |
| 36 | 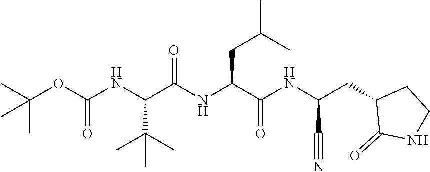 [SMILES: CC(C)C[C@H](NC(=O)[C@@H](NC(=O)OC(C)(C)C)C(C)(C)C)C(=O)N[C@H](C#N)C[C@@H]1CCNC1=O] |
| 37 | 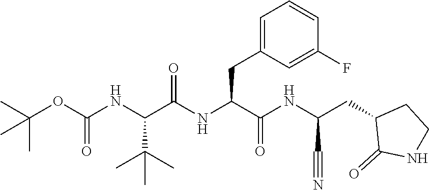 [SMILES: CC(C)(C)OC(=O)N[C@H](C(=O)N[C@@H](Cc1cccc(F)c1)C(=O)N[C@H](C#N)C[C@@H]1CCNC1=O)C(C)(C)C] |
| 38 | 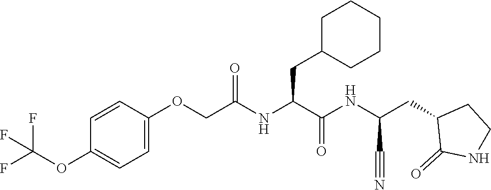 [SMILES: N#C[C@H](C[C@@H]1CCNC1=O)NC(=O)[C@H](CC1CCCCC1)NC(=O)COc1ccc(OC(F)(F)F)cc1] |
| 39 | 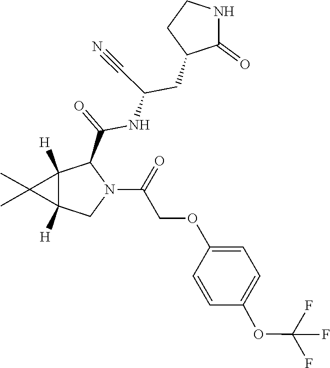 [SMILES: CC1(C)[C@@H]2[C@@H](C(=O)N[C@H](C#N)C[C@@H]3CCNC3=O)N(C(=O)COc3ccc(OC(F)(F)F)cc3)C[C@@H]21] |
| 40 | 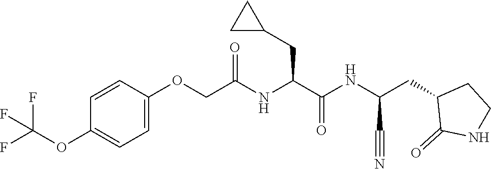 [SMILES: N#C[C@H](C[C@@H]1CCNC1=O)NC(=O)[C@H](CC1CC1)NC(=O)COc1ccc(OC(F)(F)F)cc1] |
| 41 | 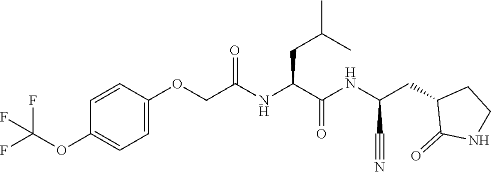 [SMILES: CC(C)C[C@H](NC(=O)COc1ccc(OC(F)(F)F)cc1)C(=O)N[C@H](C#N)C[C@@H]1CCNC1=O] |
| 42 | 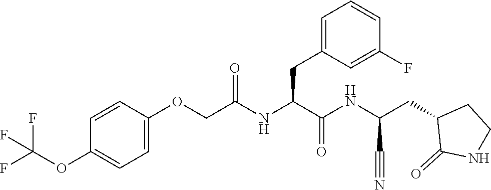 [SMILES: N#C[C@H](C[C@@H]1CCNC1=O)NC(=O)[C@H](Cc1cccc(F)c1)NC(=O)COc1ccc(OC(F)(F)F)cc1] |
| 43 | 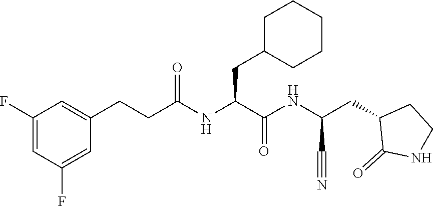 [SMILES: N#C[C@H](C[C@@H]1CCNC1=O)NC(=O)[C@H](CC1CCCCC1)NC(=O)CCc1cc(F)cc(F)c1] |
| 44 | 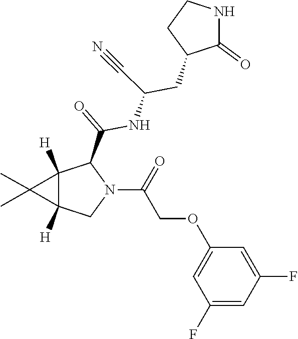 [SMILES: CC1(C)[C@@H]2[C@@H](C(=O)N[C@H](C#N)C[C@@H]3CCNC3=O)N(C(=O)COc3cc(F)cc(F)c3)C[C@@H]21] |
| 45 | 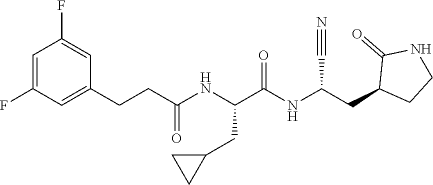 [SMILES: N#C[C@H](C[C@@H]1CCNC1=O)NC(=O)[C@H](CC1CC1)NC(=O)CCc1cc(F)cc(F)c1] |
| 46 | 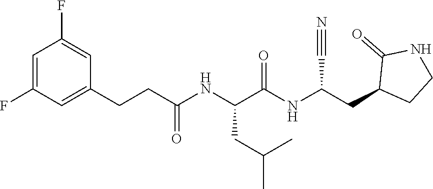 [SMILES: CC(C)C[C@H](NC(=O)CCc1cc(F)cc(F)c1)C(=O)N[C@H](C#N)C[C@@H]1CCNC1=O] |
| 47 | 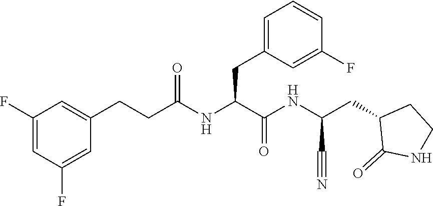 [SMILES: N#C[C@H](C[C@@H]1CCNC1=O)NC(=O)[C@H](Cc1cccc(F)c1)NC(=O)CCc1cc(F)cc(F)c1] |
| 48 | 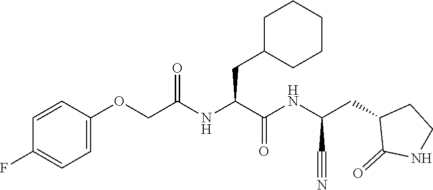 [SMILES: N#C[C@H](C[C@@H]1CCNC1=O)NC(=O)[C@H](CC1CCCCC1)NC(=O)COc1ccc(F)cc1] |
| 49 | 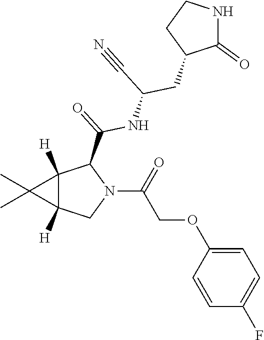 [SMILES: CC1(C)[C@@H]2[C@@H](C(=O)N[C@H](C#N)C[C@@H]3CCNC3=O)N(C(=O)COc3ccc(F)cc3)C[C@@H]21] |
| 50 | 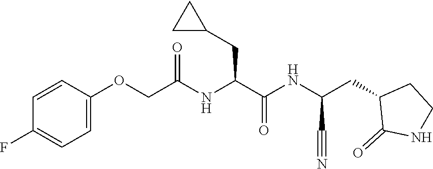 [SMILES: N#C[C@H](C[C@@H]1CCNC1=O)NC(=O)[C@H](CC1CC1)NC(=O)COc1ccc(F)cc1] |
| 51 | 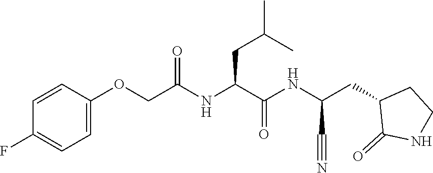 [SMILES: CC(C)C[C@H](NC(=O)COc1ccc(F)cc1)C(=O)N[C@H](C#N)C[C@@H]1CCNC1=O] |
| 52 | 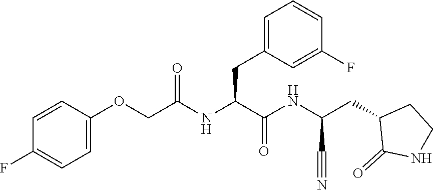 [SMILES: N#C[C@H](C[C@@H]1CCNC1=O)NC(=O)[C@H](Cc1cccc(F)c1)NC(=O)COc1ccc(F)cc1] |
| 53 | 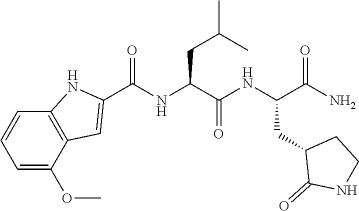 [SMILES: COc1cccc2[nH]c(C(=O)N[C@@H](CC(C)C)C(=O)N[C@@H](C[C@@H]3CCNC3=O)C(N)=O)cc12] |
| 54 | 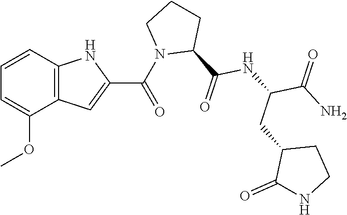 [SMILES: COc1cccc2[nH]c(C(=O)N3CCC[C@H]3C(=O)N[C@@H](C[C@@H]3CCNC3=O)C(N)=O)cc12] |
| 55 | 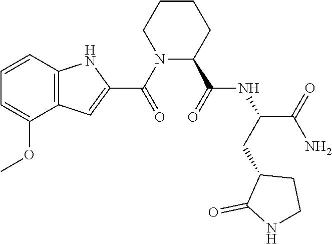 [SMILES: COc1cccc2[nH]c(C(=O)N3CCCC[C@H]3C(=O)N[C@@H](C[C@@H]3CCNC3=O)C(N)=O)cc12] |
| 56 | 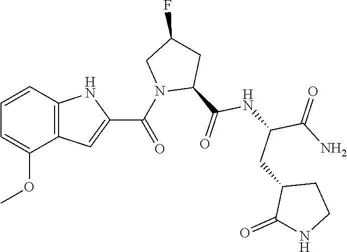 [SMILES: COc1cccc2[nH]c(C(=O)N3C[C@@H](F)C[C@H]3C(=O)N[C@@H](C[C@@H]3CCNC3=O)C(N)=O)cc12] |
| 57 | 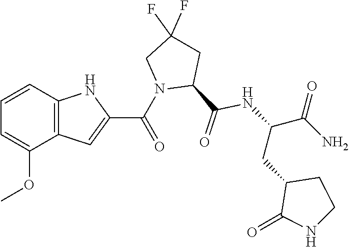 [SMILES: COc1cccc2[nH]c(C(=O)N3CC(F)(F)C[C@H]3C(=O)N[C@@H](C[C@@H]3CCNC3=O)C(N)=O)cc12] |
| 58 | 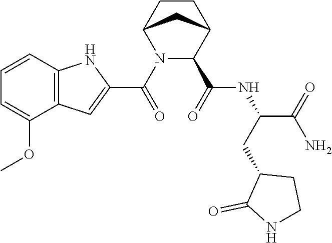 [SMILES: COc1cccc2[nH]c(C(=O)N3[C@@H]4CC[C@@H](C4)[C@H]3C(=O)N[C@@H](C[C@@H]3CCNC3=O)C(N)=O)cc12] |
| 59 | 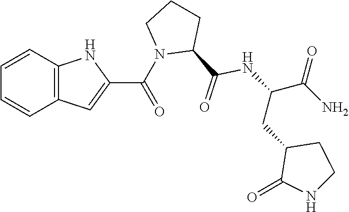 [SMILES: NC(=O)[C@H](C[C@@H]1CCNC1=O)NC(=O)[C@@H]1CCCN1C(=O)c1cc2ccccc2[nH]1] |
| 60 | 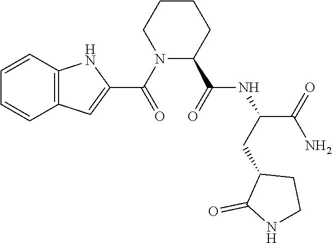 [SMILES: NC(=O)[C@H](C[C@@H]1CCNC1=O)NC(=O)[C@@H]1CCCCN1C(=O)c1cc2ccccc2[nH]1] |
| 61 | 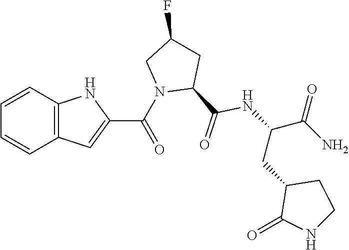 [SMILES: NC(=O)[C@H](C[C@@H]1CCNC1=O)NC(=O)[C@@H]1C[C@H](F)CN1C(=O)c1cc2ccccc2[nH]1] |
| 62 | 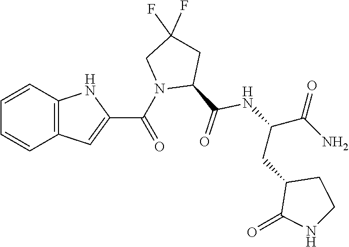 [SMILES: NC(=O)[C@H](C[C@@H]1CCNC1=O)NC(=O)[C@@H]1CC(F)(F)CN1C(=O)c1cc2ccccc2[nH]1] |
| 63 | 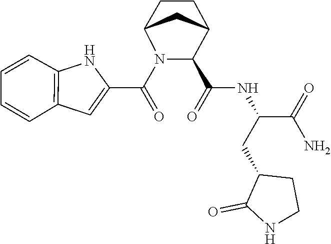 [SMILES: NC(=O)[C@H](C[C@@H]1CCNC1=O)NC(=O)[C@@H]1[C@H]2CC[C@H](C2)N1C(=O)c1cc2ccccc2[nH]1] |
| 64 | 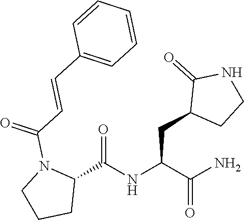 [SMILES: NC(=O)[C@H](C[C@@H]1CCNC1=O)NC(=O)[C@@H]1CCCN1C(=O)/C=C/c1ccccc1] |
| 65 | 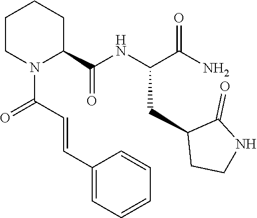 [SMILES: NC(=O)[C@H](C[C@@H]1CCNC1=O)NC(=O)[C@@H]1CCCCN1C(=O)/C=C/c1ccccc1] |
| 66 | 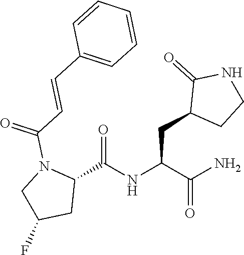 [SMILES: NC(=O)[C@H](C[C@@H]1CCNC1=O)NC(=O)[C@@H]1C[C@H](F)CN1C(=O)/C=C/c1ccccc1] |
| 67 | 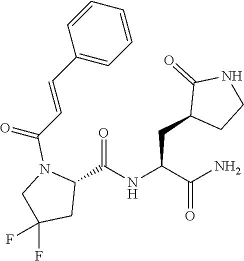 [SMILES: NC(=O)[C@H](C[C@@H]1CCNC1=O)NC(=O)[C@@H]1CC(F)(F)CN1C(=O)/C=C/c1ccccc1] |
| 68 | 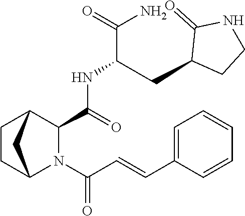 [SMILES: NC(=O)[C@H](C[C@@H]1CCNC1=O)NC(=O)[C@@H]1[C@H]2CC[C@H](C2)N1C(=O)/C=C/c1ccccc1] |
| 69 | 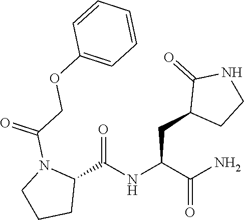 [SMILES: NC(=O)[C@H](C[C@@H]1CCNC1=O)NC(=O)[C@@H]1CCCN1C(=O)COc1ccccc1] |
| 70 | 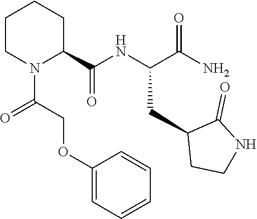 [SMILES: NC(=O)[C@H](C[C@@H]1CCNC1=O)NC(=O)[C@@H]1CCCCN1C(=O)COc1ccccc1] |
| 71 | 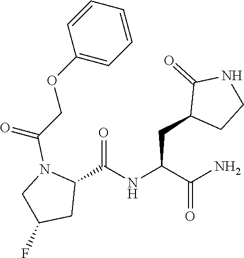 [SMILES: NC(=O)[C@H](C[C@@H]1CCNC1=O)NC(=O)[C@@H]1C[C@H](F)CN1C(=O)COc1ccccc1] |
| 72 | 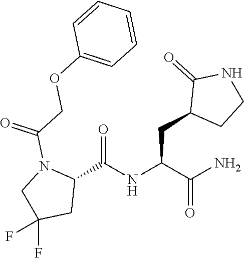 [SMILES: NC(=O)[C@H](C[C@@H]1CCNC1=O)NC(=O)[C@@H]1CC(F)(F)CN1C(=O)COc1ccccc1] |
| 73 | 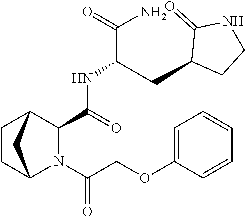 [SMILES: NC(=O)[C@H](C[C@@H]1CCNC1=O)NC(=O)[C@@H]1[C@H]2CC[C@H](C2)N1C(=O)COc1ccccc1] |
| 74 | 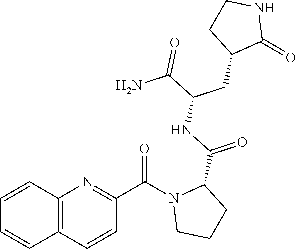 [SMILES: NC(=O)[C@H](C[C@@H]1CCNC1=O)NC(=O)[C@@H]1CCCN1C(=O)c1ccc2ccccc2n1] |
| 75 | 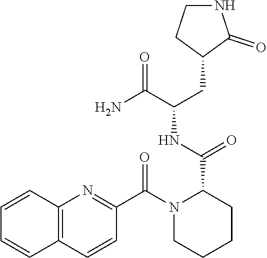 [SMILES: NC(=O)[C@H](C[C@@H]1CCNC1=O)NC(=O)[C@@H]1CCCCN1C(=O)c1ccc2ccccc2n1] |
| 76 | 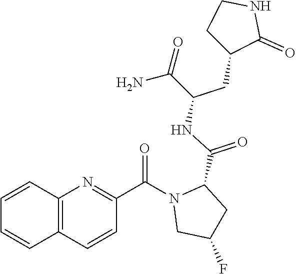 [SMILES: NC(=O)[C@H](C[C@@H]1CCNC1=O)NC(=O)[C@@H]1C[C@H](F)CN1C(=O)c1ccc2ccccc2n1] |
| 77 | 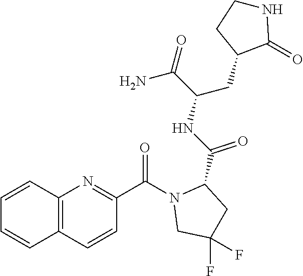 [SMILES: NC(=O)[C@H](C[C@@H]1CCNC1=O)NC(=O)[C@@H]1CC(F)(F)CN1C(=O)c1ccc2ccccc2n1] |
| 78 | 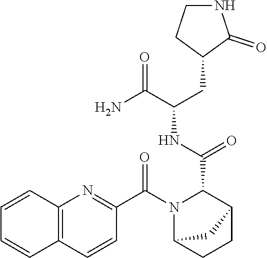 [SMILES: NC(=O)[C@H](C[C@@H]1CCNC1=O)NC(=O)[C@@H]1[C@H]2CC[C@H](C2)N1C(=O)c1ccc2ccccc2n1] |
| 79 | 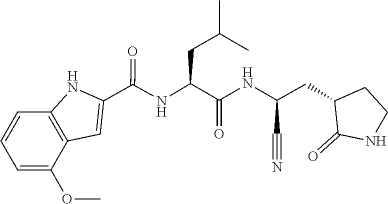 [SMILES: COc1cccc2[nH]c(C(=O)N[C@@H](CC(C)C)C(=O)N[C@H](C#N)C[C@@H]3CCNC3=O)cc12] |
| 80 | 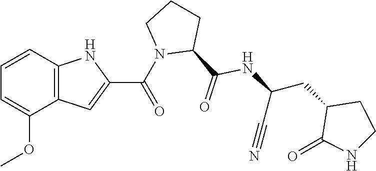 [SMILES: COc1cccc2[nH]c(C(=O)N3CCC[C@H]3C(=O)N[C@H](C#N)C[C@@H]3CCNC3=O)cc12] |
| 81 | 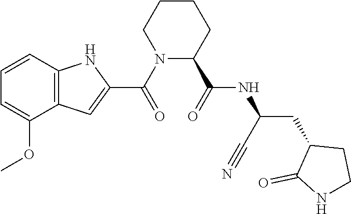 [SMILES: COc1cccc2[nH]c(C(=O)N3CCCC[C@H]3C(=O)N[C@H](C#N)C[C@@H]3CCNC3=O)cc12] |
| 82 | 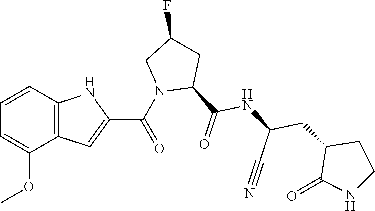 [SMILES: COc1cccc2[nH]c(C(=O)N3C[C@@H](F)C[C@H]3C(=O)N[C@H](C#N)C[C@@H]3CCNC3=O)cc12] |
| 83 | 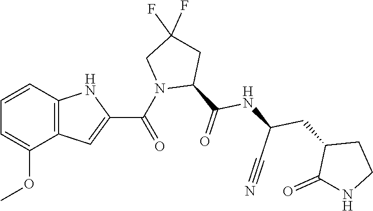 [SMILES: COc1cccc2[nH]c(C(=O)N3CC(F)(F)C[C@H]3C(=O)N[C@H](C#N)C[C@@H]3CCNC3=O)cc12] |
| 84 | 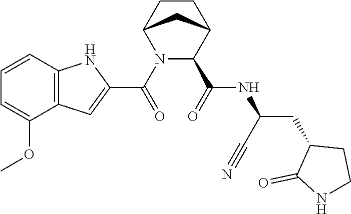 [SMILES: COc1cccc2[nH]c(C(=O)N3[C@@H]4CC[C@@H](C4)[C@H]3C(=O)N[C@H](C#N)C[C@@H]3CCNC3=O)cc12] |
| 85 | 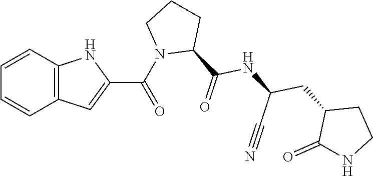 [SMILES: N#C[C@H](C[C@@H]1CCNC1=O)NC(=O)[C@@H]1CCCN1C(=O)c1cc2ccccc2[nH]1] |
| 86 | 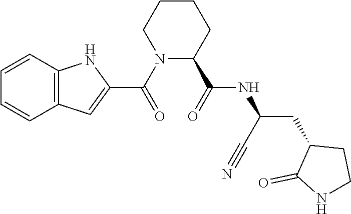 [SMILES: N#C[C@H](C[C@@H]1CCNC1=O)NC(=O)[C@@H]1CCCCN1C(=O)c1cc2ccccc2[nH]1] |
| 87 | 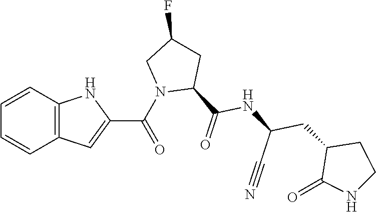 [SMILES: N#C[C@H](C[C@@H]1CCNC1=O)NC(=O)[C@@H]1C[C@H](F)CN1C(=O)c1cc2ccccc2[nH]1] |
| 88 | 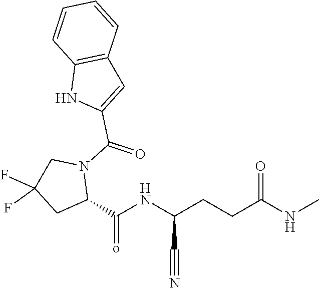 |
| 89 | 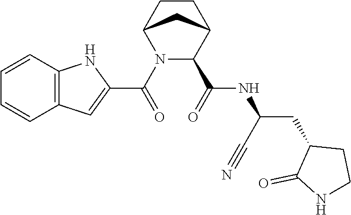 [SMILES: N#C[C@H](C[C@@H]1CCNC1=O)NC(=O)[C@@H]1[C@H]2CC[C@H](C2)N1C(=O)c1cc2ccccc2[nH]1] |
| 90 | 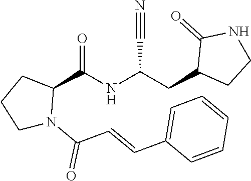 |
| 91 | 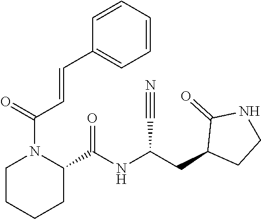 [SMILES: N#C[C@H](C[C@@H]1CCNC1=O)NC(=O)[C@@H]1CCCCN1C(=O)/C=C/c1ccccc1] |
| 92 | 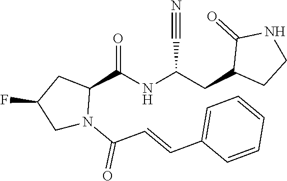 |
| 93 | 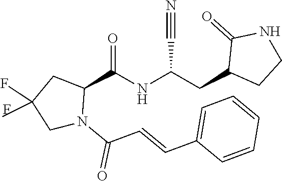 |
| 94 | 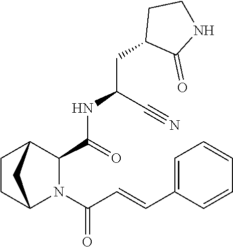 [SMILES: N#C[C@H](C[C@@H]1CCNC1=O)NC(=O)[C@@H]1[C@H]2CC[C@H](C2)N1C(=O)/C=C/c1ccccc1] |
| 95 | 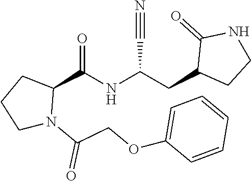 |
| 96 | 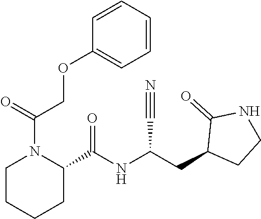 [SMILES: N#C[C@H](C[C@@H]1CCNC1=O)NC(=O)[C@@H]1CCCCN1C(=O)COc1ccccc1] |
| 96-1 | 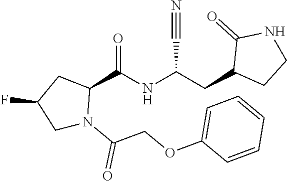 |
| 96-2 | 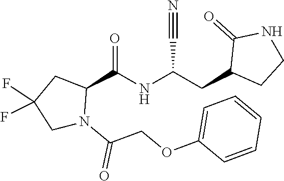 |
| 96-3 | 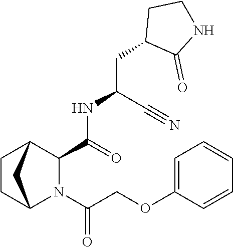 [SMILES: N#C[C@H](C[C@@H]1CCNC1=O)NC(=O)[C@@H]1[C@H]2CC[C@H](C2)N1C(=O)COc1ccccc1] |
| 96-4 | 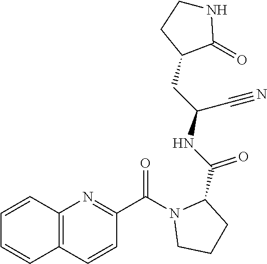 [SMILES: N#C[C@H](C[C@@H]1CCNC1=O)NC(=O)[C@@H]1CCCN1C(=O)c1ccc2ccccc2n1] |
| 96-5 | 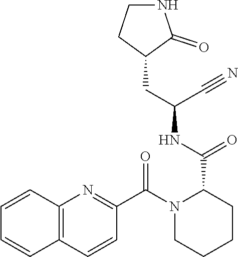 [SMILES: N#C[C@H](C[C@@H]1CCNC1=O)NC(=O)[C@@H]1CCCCN1C(=O)c1ccc2ccccc2n1] |
| 96-6 | 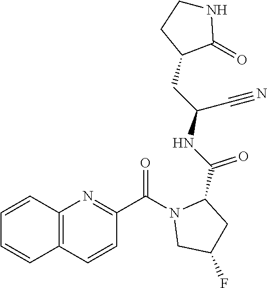 [SMILES: N#C[C@H](C[C@@H]1CCNC1=O)NC(=O)[C@@H]1C[C@H](F)CN1C(=O)c1ccc2ccccc2n1] |
| 96-7 | 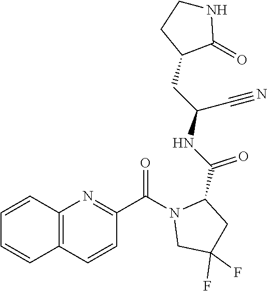 [SMILES: N#C[C@H](C[C@@H]1CCNC1=O)NC(=O)[C@@H]1CC(F)(F)CN1C(=O)c1ccc2ccccc2n1] |
| 96-8 | 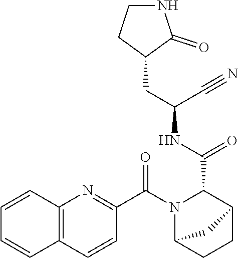 [SMILES: N#C[C@H](C[C@@H]1CCNC1=O)NC(=O)[C@@H]1[C@H]2CC[C@H](C2)N1C(=O)c1ccc2ccccc2n1] |
| 97 (prior art) | 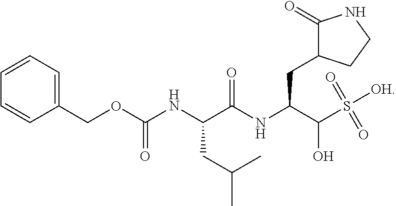 [SMILES: CC(C)C[C@H](NC(=O)OCc1ccccc1)C(=O)N[C@@H](CC1CCNC1=O)C(O)S(=O)(=O)O] |
| 98 | 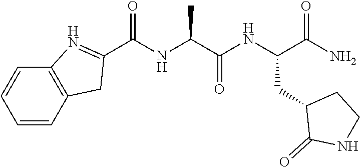 |
| 99 | 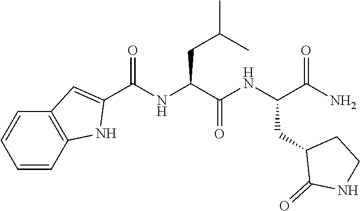 [SMILES: CC(C)C[C@H](NC(=O)c1cc2ccccc2[nH]1)C(=O)N[C@@H](C[C@@H]1CCNC1=O)C(N)=O] |
| 100 | 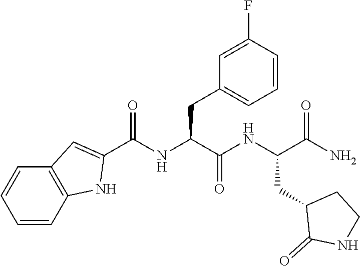 [SMILES: NC(=O)[C@H](C[C@@H]1CCNC1=O)NC(=O)[C@H](Cc1cccc(F)c1)NC(=O)c1cc2ccccc2[nH]1] |
| 101 | 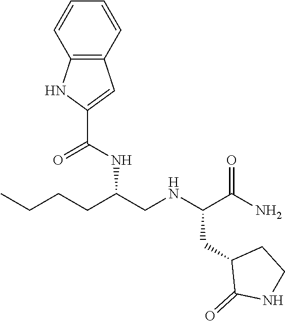 [SMILES: CCCC[C@@H](CN[C@@H](C[C@@H]1CCNC1=O)C(N)=O)NC(=O)c1cc2ccccc2[nH]1] |
| 102 | 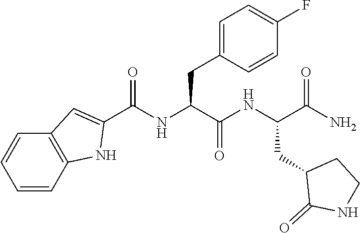 [SMILES: NC(=O)[C@H](C[C@@H]1CCNC1=O)NC(=O)[C@H](Cc1ccc(F)cc1)NC(=O)c1cc2ccccc2[nH]1] |
| 103 | 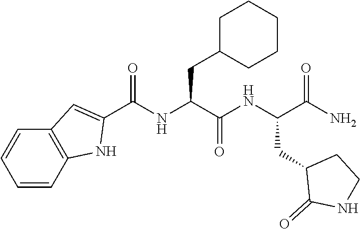 [SMILES: NC(=O)[C@H](C[C@@H]1CCNC1=O)NC(=O)[C@H](CC1CCCCC1)NC(=O)c1cc2ccccc2[nH]1] |
| 104 | 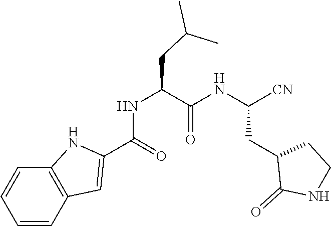 [SMILES: CC(C)C[C@H](NC(=O)c1cc2ccccc2[nH]1)C(=O)N[C@H](C#N)C[C@@H]1CCNC1=O] |
| 105 | 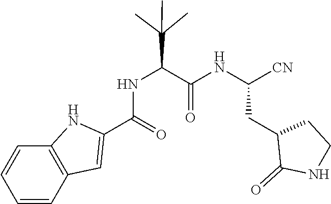 [SMILES: CC(C)(C)[C@H](NC(=O)c1cc2ccccc2[nH]1)C(=O)N[C@H](C#N)C[C@@H]1CCNC1=O] |
| 106 | 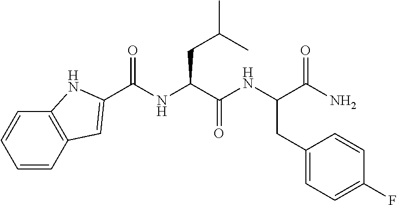 [SMILES: CC(C)C[C@H](NC(=O)c1cc2ccccc2[nH]1)C(=O)NC(Cc1ccc(F)cc1)C(N)=O] |
| 107 | 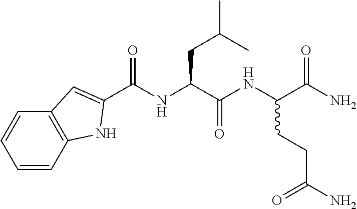 [SMILES: CC(C)C[C@H](NC(=O)c1cc2ccccc2[nH]1)C(=O)NC(CCC(N)=O)C(N)=O] |
| 108 | 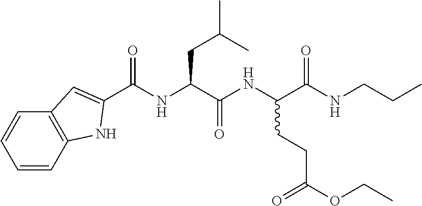 [SMILES: CCCNC(=O)C(CCC(=O)OCC)NC(=O)[C@H](CC(C)C)NC(=O)c1cc2ccccc2[nH]1] |
| 109 | 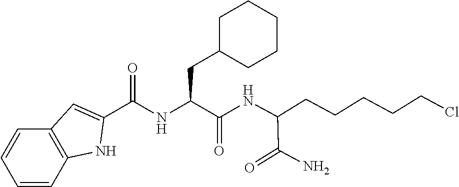 [SMILES: NC(=O)C(CCCCCCl)NC(=O)[C@H](CC1CCCCC1)NC(=O)c1cc2ccccc2[nH]1] |
| 110 | 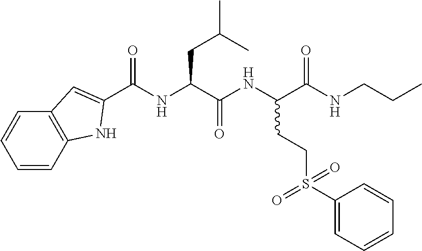 [SMILES: CCCNC(=O)C(CCS(=O)(=O)c1ccccc1)NC(=O)[C@H](CC(C)C)NC(=O)c1cc2ccccc2[nH]1] |
| 111 | 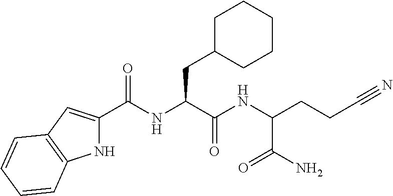 [SMILES: N#CCCC(NC(=O)[C@H](CC1CCCCC1)NC(=O)c1cc2ccccc2[nH]1)C(N)=O] |
| 112 | 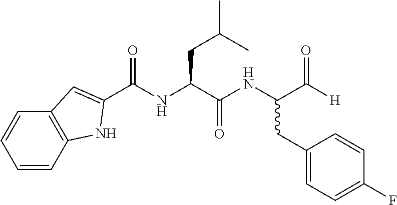 [SMILES: CC(C)C[C@H](NC(=O)c1cc2ccccc2[nH]1)C(=O)NC(C=O)Cc1ccc(F)cc1] |
| 113 | 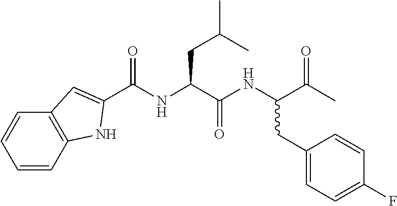 [SMILES: CC(=O)C(Cc1ccc(F)cc1)NC(=O)[C@H](CC(C)C)NC(=O)c1cc2ccccc2[nH]1] |
| 114 | 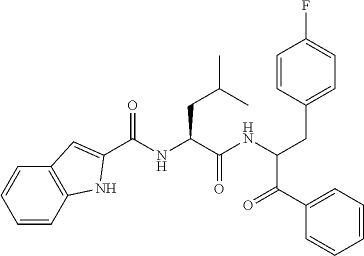 [SMILES: CC(C)C[C@H](NC(=O)c1cc2ccccc2[nH]1)C(=O)NC(Cc1ccc(F)cc1)C(=O)c1ccccc1] |
| 115 | 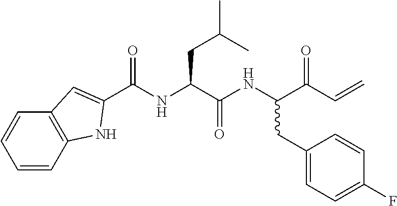 [SMILES: C=CC(=O)C(Cc1ccc(F)cc1)NC(=O)[C@H](CC(C)C)NC(=O)c1cc2ccccc2[nH]1] |
| 116 | 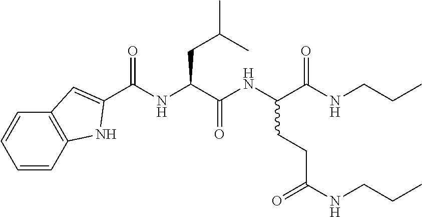 [SMILES: CCCNC(=O)CCC(NC(=O)[C@H](CC(C)C)NC(=O)c1cc2ccccc2[nH]1)C(=O)NCCC] |
| 117 | 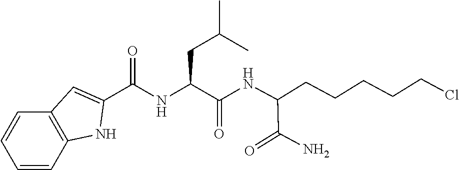 [SMILES: CC(C)C[C@H](NC(=O)c1cc2ccccc2[nH]1)C(=O)NC(CCCCCCl)C(N)=O] |
| 118 | 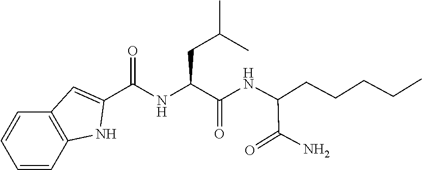 [SMILES: CCCCCC(NC(=O)[C@H](CC(C)C)NC(=O)c1cc2ccccc2[nH]1)C(N)=O] |
| 119 | 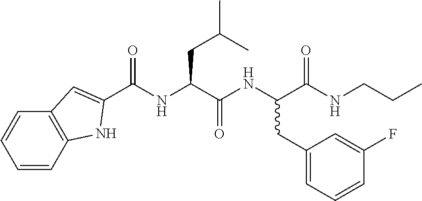 [SMILES: CCCNC(=O)C(Cc1cccc(F)c1)NC(=O)[C@H](CC(C)C)NC(=O)c1cc2ccccc2[nH]1] |
| 120 | 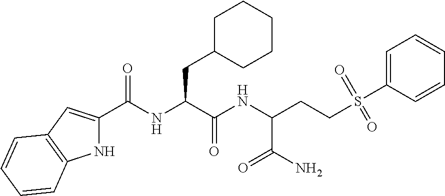 [SMILES: NC(=O)C(CCS(=O)(=O)c1ccccc1)NC(=O)[C@H](CC1CCCCC1)NC(=O)c1cc2ccccc2[nH]1] |
| 121 | 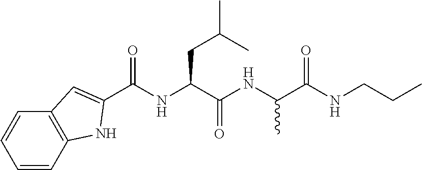 [SMILES: CCCNC(=O)C(C)NC(=O)[C@H](CC(C)C)NC(=O)c1cc2ccccc2[nH]1] |
| 122 | 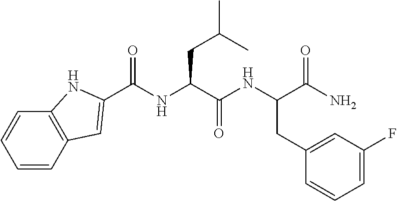 [SMILES: CC(C)C[C@H](NC(=O)c1cc2ccccc2[nH]1)C(=O)NC(Cc1cccc(F)c1)C(N)=O] |
| 123 | 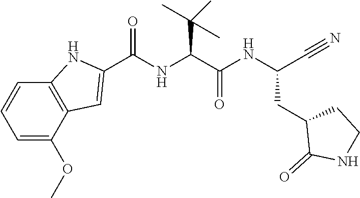 [SMILES: COc1cccc2[nH]c(C(=O)N[C@H](C(=O)N[C@H](C#N)C[C@@H]3CCNC3=O)C(C)(C)C)cc12] |
| 124 | 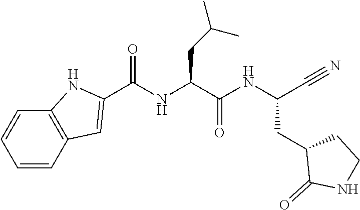 [SMILES: CC(C)C[C@H](NC(=O)c1cc2ccccc2[nH]1)C(=O)N[C@H](C#N)C[C@@H]1CCNC1=O] |
| 125 | 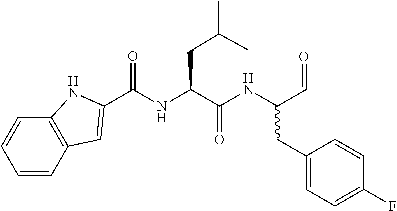 [SMILES: CC(C)C[C@H](NC(=O)c1cc2ccccc2[nH]1)C(=O)NC(C=O)Cc1ccc(F)cc1] |
| 126 | 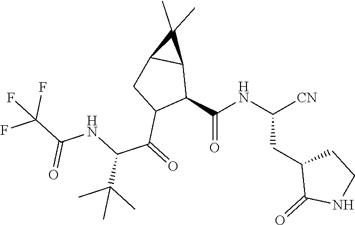 [SMILES: CC(C)(C)[C@H](NC(=O)C(F)(F)F)C(=O)C1C[C@@H]2[C@H]([C@H]1C(=O)N[C@H](C#N)C[C@@H]1CCNC1=O)C2(C)C] |
| 127 | 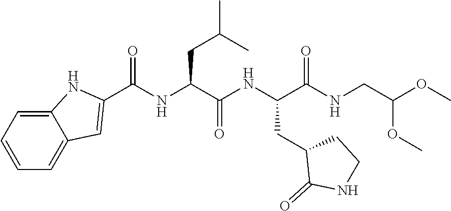 [SMILES: COC(CNC(=O)[C@H](C[C@@H]1CCNC1=O)NC(=O)[C@H](CC(C)C)NC(=O)c1cc2ccccc2[nH]1)OC] |
| 128 | 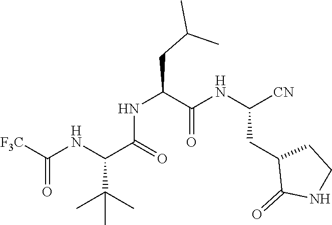 [SMILES: CC(C)C[C@H](NC(=O)[C@@H](NC(=O)C(F)(F)F)C(C)(C)C)C(=O)N[C@H](C#N)C[C@@H]1CCNC1=O] |

### Pharmaceutical Compositions

The compositions and methods of the present disclosure may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the disclosure and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the disclosure. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the disclosure. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (or preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the disclosure, with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present disclosure to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this disclosure, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with a compound of the disclosure. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the disclosure will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present disclosure, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the disclosure may be used alone or conjointly administered with another type of therapeutic agent.

The present disclosure includes the use of pharmaceutically acceptable salts of compounds of the disclosure in the compositions and methods of the present disclosure. In certain embodiments, contemplated salts of the disclosure include, but are not limited to, alkyl, dialkyl, trialkyl or tetra-alkyl ammonium salts. In certain embodiments, contemplated salts of the disclosure include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the disclosure include, but are not limited to, Na, Ca, K, Mg, Zn or other metal salts. In certain embodiments, contemplated salts of the disclosure include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

In certain embodiments, the present invention provides pharmaceutical compositions comprising a compound described herein, such as a compound of Formula I and II. In certain embodiments, the pharmaceutical compositions further comprise a pharmaceutically acceptable excipient.

In certain embodiments, the pharmaceutical compositions may be for use in treating or preventing a condition or disease as described herein.

The compounds described herein are useful, for example, as therapeutics for the treatment of diseases. In certain aspects, the present disclosure provides a pharmaceutical composition comprising a compound of the disclosure and a pharmaceutically acceptable excipient.

In certain aspects, the present disclosure provides methods of treating a viral infection caused by a Sarbecovirus or SARS-CoV virus or a mutation thereof. In some embodiments, the virus causes COVID.

In certain aspects, the compounds and pharmaceutical compositions of the disclosure specifically target a main protease (Mpro). Thus, these compounds and pharmaceutical compositions can be used to prevent, reverse, slow, or inhibit the activity of Mpro.

In certain embodiments, compounds of the invention are prodrugs of the compounds described herein. For example, wherein a hydroxyl in the parent compound is presented as an ester or a carbonate, or a carboxylic acid present in the parent compound is presented as an ester. In certain such embodiments, the prodrug is metabolized to the active parent compound in vivo (e.g., the ester is hydrolyzed to the corresponding hydroxyl or carboxylic acid).

In certain embodiments, compounds of the invention may be racemic. In certain embodiments, compounds of the invention may be enriched in one enantiomer. For example, a compound of the invention may have greater than 30% ee, 40% ee, 50% ee, 60% ee, 70% ee, 80% ee, 90% ee, or even 95% or greater ee. In certain embodiments, compounds of the invention may have more than one stereocenter. In certain such embodiments, compounds of the invention may be enriched in one or more diastereomers. For example, a compound of the invention may have greater than 30% de, 40% de, 50% de, 60% de, 70% de, 80% de, 90% de, or even 95% or greater de.

### Chemical Synthesis Methods

The following examples are offered to illustrate but not to limit the disclosure. One of skill in the art will recognize that the following synthetic reactions and schemes may be modified by choice of suitable starting materials and reagents in order to access other compounds of Formula I and II.

Abbreviations: The examples described herein use materials, including but not limited to, those described by the following abbreviations known to those skilled in the art:

| | |
|---|---|
| g | grams |
| eq | equivalents |
| mmol | millimoles |
| mL | milliliters |
| EtOAc | ethyl acetate |
| MHz | megahertz |
| ppm | parts per million |
| CDCl₃ | deuterated chloroform |
| δ | chemical shift |
| s | singlet |
| d | doublet |
| t | triplet |
| q | quartet |
| quin | quintet |
| br | broad |
| m | multiplet |
| Hz | hertz |
| THF | tetrahydrofuran |
| ° C. | degrees Celsius |
| PE | petroleum ether |
| EA | ethyl acetate |
| R_{f} | retardation factor |
| N | normal |
| J | coupling constant |
| DMSO-d₆ | deuterated dimethyl sulfoxide |
| n-BuOH | n-butanol |
| DIEA | n,n-diisopropylethylamine |
| TMSCl | trimethylsilyl chloride |
| min | minutes |
| hr | hours |
| Me | methyl |
| Et | ethyl |
| i-Pr | isopropyl |
| TLC | thin layer chromatography |
| M | molar |
| Compd# | compound number |
| MS | mass spectrum |
| ESI | Electrospray ionization |
| m/z | mass-to-charge ratio |
| Ms | methanesulfonyl |
| Boc | tert-butyloxycarbonyl |
| TFA | trifluoroacetic acid |
| Tos | toluenesulfonyl |
| DMAP | 4-(dimethylamino)pyridine |
| mM | micromolar |
| ATP | adenosine triphosphate |
| IC₅₀ | half maximal inhibitory concentration |
| U/mL | units of activity per milliliter |
| DIAD | diisopropyl azodicarboxylate |
| DMSO | dimethyl sulfoxide |
| MeTHF | 2-methyltetrahydrofuran |
| MOM | methoxymethyl |
| DCM | dichloromethane |
| DMF | N,N-dimethylformamide |
| DBU | 1,8-diazabicyclo[5.4.0]undec-7-ene |
| DIPEA | N,N-diisopropylethylamine |
| Hex | hexanes |
| Pd(dppf)Cl₂ | [1,1′-Bis(diphenylphosphino)ferrocene]dichloro- |
| | palladium(II) |
| MeCN (ACN) | Acetonitrile |
| Hunig's Base | N,N-diisopropylethylamine |
| TBAF | Tert butyl ammonium fluoride |
| HBTU | (2-(1H-benzotriazol-1-yl)-1,1,3,3-tetramethyluronium |
| | hexafluorophosphate |
| PPh₃ | Triphenyl phosphine |
| RT | Room Temperature |
| p-TSA | Para-Tolylsulfonic acid |
| t-BuOH | Tert-Butanol |
| mCPBA | Meta-Chloroperoxy benzoic acid |
| AcOH | Acetic Acid |
| DMAc | N,N-Dimethylformamide |

The proposed targets can be prepared via the conventional chemistry or following the general schemes as shown below.

### Synthesis of Mpro Inhibitors

### Primary Amides (Mpro Inhibitor Intermediates):

Reagents were purchased as at least reagent grade from Aldrich, Acros, Alfa Aesar or other vendors and used without further purification. Compounds were synthesized according to general methods described below, and purity determined using HPLC.

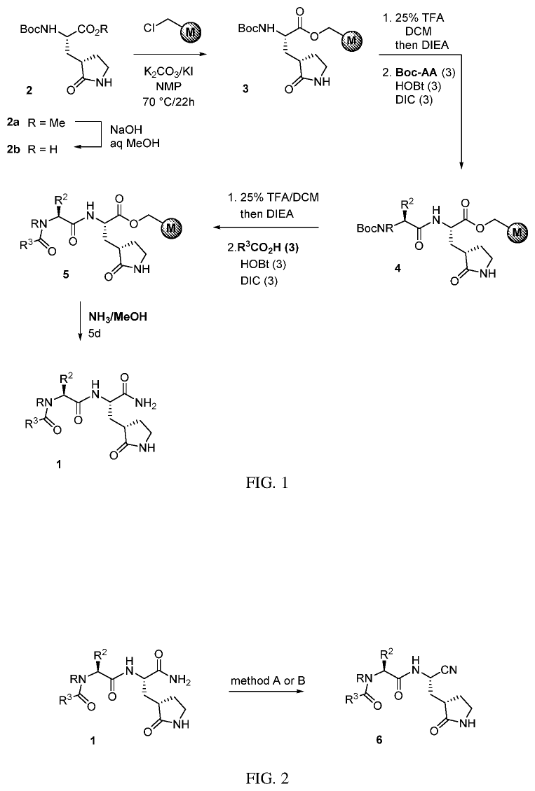

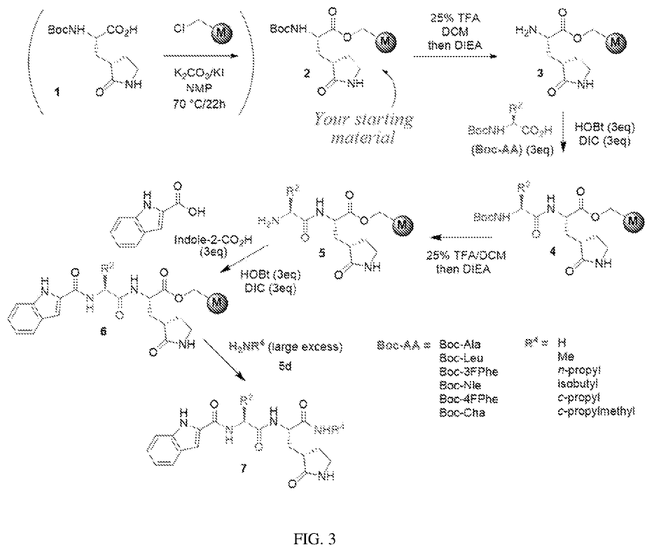

Turning to FIG. 1 shows an illustration of the synthetic scheme for the primary amides. See FIG. 3 for another example of the synthesis scheme of the primary amides.

(S)-2-((tert-Butoxycarbonyl)amino)-3-((S)-2-oxopyrrolidin-3-yl)propanoic acid (2b). A solution of 7.67 g (26.8 mmol) of 2a in 33 mL of methanol was cooled to 0-3° C. and 36.0 mL (108 mmol, 4.03 equiv) of 3.0M sodium hydroxide (pre-cooled in an ice bath) was added dropwise over 45 minutes. After 75 minutes the pH was adjusted to 5-6 using cold, concentrated HCl. Volatiles were removed in vacuo and the remaining solution was transferred to a 250-mL beaker using ethyl acetate/water (30 mL/15 mL). The pH was adjusted to approx. 3 with 1.0N HCl. The layers were separated and the aqueous phase was extracted with 2×15 mL of ethyl acetate. The combined organics were washed once with 3 mL of saturated brine and were dried (MgSO₄). Concentration gave 6.68 g (91% by weight, 83% yield) of 2b; ¹HNMR (CDCl₃) δ 1.47 (s, 9H), 1.87-1.98 (m, 2H), 2.20-2.25 (m, 1H), 2.43-2.51 (m, 1H), 2.66 (quintet, 1H, J=7.9 Hz), 3.39-3.50 (2m, 2H), 4.42 (br q, 1H, J=7.0 Hz), 5.72 (br d, 1H, J=7.5 Hz), 6.84 (br s, 1H).

Alkylation of Merrifield resin with 2b to give loaded Merrifield resin 3 (Boc-c-Gln-Merrifield). A 500-mL, 3-neck, round-bottomed flask fitted with a stir shaft and bearing, gas inlet tube carrying dry argon gas, and a glass stopper was charged with 8.944 g (8.050 mmol) of Merrifield resin (0.9 mmol/g, 100-200 mesh). N-Methylpyrrolidinone (NMP, 100 mL) was added with stirring to fully mix the contents. Stirring was stopped and 6.675 g (22.3 mmol, 2.77 equiv.) of 91% by wt 2b in 30 mL of NMP was added. The contents were then stirred briefly to fully mix the contents. To this mixture was added 3.10 g (22.4 mmol, 2.78 equiv) of potassium carbonate followed by 334 mg (2.01 mmol, 0.25 equiv.) of potassium iodide. While being stirred slowly, the mixture was heated at 70° C. overnight. After 24 h the mixture was allowed to cool and was transferred using NMP as a rinse to a 500-mL solid-phase peptide synthesis (SPPS) vessel. Using house vacuum, the vessel was drained and the resin was washed with 3×60 mL each of NMP, 1:1 NMP/DCM, DCM, 1:1 DCM/MeOH, MeOH, 1:1 MeOH/water, water, 1:1 MeOH/water, MeOH, 1:1 DCM/MeOH, and 5×60 mL of DCM. The resin was then dried overnight under a slow stream of dry nitrogen gas to give 11.1 g (Th. 10.83 g) of resin 3 (Boc-c-Gln-Merrifield).

Boc removal and acylation of Merrifield resin 3 to give Merrifield resin 4 (Boc-AA-c-Gln-Merrifield). Resin 3 (50 mmol of Boc-c-Gln on Merrifield resin), contained in a 3.5-mL SPPS vessel, was washed with 3×2 mL of DCM. Trifluoroacetic acid (30% (v/v) TFA in DCM, 1.0 mL) was added to the resin allowing excess reagent to drip from the vessel. The resin was allowed to stand for 20 minutes and was then treated in like manner with 1.0 mL of 30% TFA/DCM. After standing for an additional 25 minutes the resin was washed with 3×2 mL of DCM, was neutralized with 4×2 mL of 10% (v/v) diisopropylethyl-amine (DIEA) in DCM, and was washed with 3×2 mL of NMP. The deprotected, free base resin was then treated with 0.60 mL (150 mmol, 3.0 equiv.) of 0.25M Boc-protected amino acid Boc-AA in 0.25M (150 mmol, 3.0 equiv.) HOBt in NMP followed by 0.30 mL (150 mmol, 3.0 equiv.) of 0.5M diisopropyl-carbodiimide in NMP. The contents were mixed and allowed to stand for 48 h. The vessel was drained, and the resin was washed with 2×2 mL of NMP and 4×2 mL of DCM to give 4 (Boc-AA-c-Gln-Merrifield).

Boc removal and acylation of Merrifield resin 4 to give Merrifield resin 5 (R³CO-AA-c-Gln-Merrifield). Resin 4 (50 mmol of R³CO-AA-c-Gln on Merrifield resin), contained in a 3.5-mL uncapped SPPS vessel, was treated with 1.0 mL of 30% TFA/DCM. The resin was allowed to stand for 20 minutes and was then treated in like manner with 1.0 mL of 30% TFA/DCM. After standing for an additional 25 minutes the resin was washed with 3×2 mL of DCM, was neutralized with 4×2 mL of 10% (v/v) diisopropylethyl-amine (DIPEA) in DCM and was washed with 3×2 mL of NMP. The deprotected, free base resin was then treated with 0.60 mL (150 mmol, 3.0 equiv.) of 0.25M R³CO₂H in 0.25M (150 mmol, 3.0 equiv.) HOBt in NMP followed by 0.30 mL (150 mmol, 3.0 equiv.) of 0.5M diisopropylcarbodiimide in NMP. The contents were mixed and allowed to stand for 48 h. The vessel was drained, and the resin was washed with 2×2 mL of NMP and 4×2 mL of THF to give resin 5 (R³CO-AA-c-Gln-Merrifield).

Ammonolytic cleavage of 1 from Merrifield resin 4. To 50 mmol of resin 5 contained in a 3.5-mL SPPS vessel was added 2 mL of 7N ammonia in methanol. The contents were allowed to stand with occasional agitation for five days. The vessel was drained into a pre-weighed 4-dr vial and the resin was washed with 3×2 mL of THF, collecting the filtrates in the 4-dr vial (a portion, 120 mL, was removed for LCMS analysis). The solution was evaporated to dryness to give crude primary amide 1. Crude primary amides 1 were purified by chromatography, recrystallization, or trituration as indicated in the following characterizations.

### Characterization of Amides

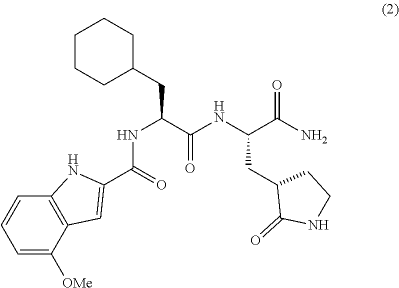

N—((S)-1-(((S)-1-amino-1-oxo-3-((S)-2-oxopyrrolidin-3-yl)propan-2-yl)amino)-3-cyclohexyl-1-oxopropan-2-yl)-4-methoxy-1H-indole-2-carboxamide (Cmpd No. 2). The crude material was dissolved in 500 mL of pure methanol and dried with nitrogen followed by ˜1 mL of chloroform and was dispensed onto a 500-mg HyperSep SI cartridge of silica gel which had been equilibrated with chloroform. Elution with chloroform/methanol (0-5% methanol) in step gradient manner afforded 30.9 mg (49.8%) of S2; ¹HNMR (methanol-d₄), d 0.86-1.07 (m, 2H), 1.10-1.36 (m, 3H), 1.41-1.55 (m, 1H), 1.57-1.89 (m, 9H), 2.171 (ddd, 1H, J=15.6, 11.4, and 4.6 Hz), 2.29 (dddd, 1H, J=15.2, 11.3, 9.1 and 2.6 Hz), 2.53 (ddd, 1H, J=14.0, 9.6 and J=4.7 Hz), 3.2-3.3 (m, 2H), 3.9 (s, 3H), 4.5 (dd, 1H, J=11.2 and 4.3 Hz), 4.67 (t, 1H, J=7.6 Hz), 6.51 (d, 1H, J=7.7 Hz), 7.04 (d, 1H, J=8.3 Hz), 7.15 (t, 1H, J=8.0 Hz), 7.32 (s, 1H); ¹³CNMR (methanol-d₄), d 25.8, 26.0, 26.2, 27.4, 32.1, 33.1, 33.4, 34.2, 38.4, 38.7, 40.1, 51.5, 51.8, 54.3, 99.0, 101.8, 104.8, 118.7, 125.0, 128.8, 138.4, 154.2, 162.9, 174.1, 175.1, 180.6.

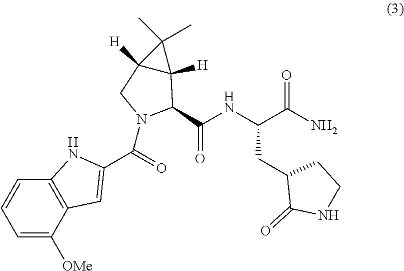

(1R,2S,5S)—N—((S)-1-Amino-1-oxo-3-((S)-2-oxopyrrolidin-3-yl)propan-2-yl)-3-(4-methoxy-1H-indole-2-carbonyl)-6,6-dimethyl-3-azabicyclo[3.1.0]hexane-2-carboxamide (Cmpd No. 3). Samples from two, 50-mmol replicate syntheses were combined and were purified by silica gel chromatography using chloroform/methanol (0-3% methanol step gradient) to afford 24.3 mg (50% from resin 3) after overnight drying at 40° C. under house vacuum: ¹HNMR (methanol-d₄), rotamer ratio 53:47, line list given for the dominant rotamer, d 0.97 (s, 3H), 1.10 (s, 3H), 1.30-1.37 (m, 1H), 1.60 (d, 1H, J=7.7 Hz), 1.69 (dd, 1H, J=7.3 and 5.5 Hz), 1.80-1.89 (m, 1H), 2.12-2.22 (m, 1H), 2.31-2.37 (m, 1H), 2.62 (ddd, 1H, J=14.3, 9.5, and 4.9 Hz), 3.27-3.31 (m, 2H), 3.80 (d, 1H, J=12.3 Hz), 3.96 (s, 3H), 4.02 (dd, 1H, J=12.3 and 5.4 Hz), 4.48 (dd, 1H, J=11.7 and 4.1 Hz), 4.62 (s, 1H), 6.53 (d, 1H, J=7.7 Hz), 7.15-7.20 (m, 3H); ¹³CNMR (methanol-d₄), line list given for the dominant rotamer, d 11.5, 19.0, 24.9, 25.1, 28.2, 30.1, 33.8, 38.4, 40.2, 48.7, 51.7, 54.3, 62.4, 98.8, 104.0, 104.7, 119.0, 125.4, 128.2, 137.5, 154.4, 161.5, 173.1, 175.3, 180.8.

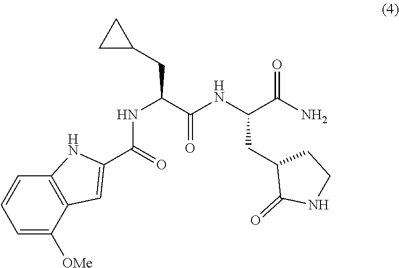

N—((S)-1-(((S)-1-amino-1-oxo-3-((S)-2-oxopyrrolidin-3-yl)propan-2-yl)amino)-3-cyclopropyl-1-oxopropan-2-yl)-4-methoxy-1H-indole-2-carboxamide (Cmpd No. 4) The crude material purified by silica gel chromatography on a 500-mg HyperSep SI cartridge. Elution with chloroform/methanol (0-4% methanol) in step gradient manner afforded 16.3 mg (36% from resin 3) of 4; ¹HNMR (methanol-d₄), d −0.031-0.078 (m, 2H), 0.28-0.39 (m, 2H), 0.66-0.76 (m, 1H), 1.54-1.72 (2m, 4H), 2.00 (ddd, 1H, J=14.2, 11.3, and 4.5 Hz), 2.15 (dddd, 1H, J=15.4, 11.7, 9.0, and 2.9 Hz), 2.39 (ddd, 1H, J=14.0, 9.6, and 4.6 Hz), 3.04-3.14 (m, 2H), 3.76 (s, 3H), 4.33 (dd, 1H, J=11.3 and 4.2 Hz), 4.46 (dd, 1H, J=8.2 and 6.1 Hz), 6.34 (d, 1H, J=7.7 Hz), 6.87 (d, 1H, J=8.3 Hz), 6.98 (t, 1H, J=8.0 Hz), 7.13 (s, 1H); ¹³CNMR (methanol-d₄) d 0.091, 1.4, 4.9, 25.0, 30.7, 33.8, 35.9, 37.6, 49.0, 51.8, 52.3, 96.4, 99.2, 102.3, 116.2, 122.5, 126.4, 135.9, 151.8, 166.3, 171.0, 172.6, 178.2.

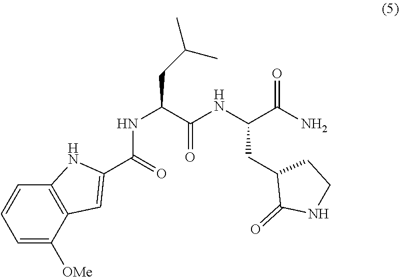

N—((S)-1-(((S)-1-amino-1-oxo-3-((S)-2-oxopyrrolidin-3-yl)propan-2-yl)amino)-4-methyl-1-oxopentan-2-yl)-4-methoxy-1H-indole-2-carboxamide (Cmpd No. 5). The crude material was dissolved in 200 mL of 95/5 chloroform/methanol and was dispensed onto a 500-mg HyperSep SI cartridge of silica gel which had been equilibrated with chloroform. Elution with chloroform/methanol (0-10% methanol) in step gradient manner afforded 12.2 mg (53% from resin 3) of 5; ¹HNMR (methanol-d₄), d 1.00 (d, 3H, J=6.3 Hz), 1.04 (d, 3H, J=6.2 Hz), 1.71-1.88 (m, 5H), 2.17 (ddd, 1H, J=14.0, 11.4, and 4.6 Hz), 2.32 (dddd, 1H, J=11.3, 9.7, 8.6 and 2.8 Hz), 2.54 (ddd, 1H, J=14.3, 9.8, and 4.6 Hz), 3.23-3.32 (m, 2H), 3.95 (s, 3H), 4.47 (dd, 1H, J=11.4 and 4.2 Hz), 4.61 (dd, 1H, J=9.9 Hz and 5.1 Hz), 6.53 (d, 1H, J=7.6 Hz), 7.05 (d, 1H, J=8.3 Hz), 7.17 (t, 1H, J=8.0 Hz), 7.31 (d, 1H, J=0.8 Hz).

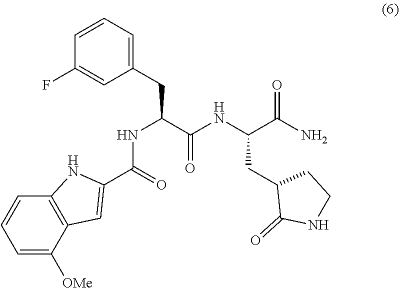

N—((S)-1-(((S)-1-amino-1-oxo-3-((S)-2-oxopyrrolidin-3-yl)propan-2-yl)amino)-3-(3-fluorophenyl)-1-oxopropan-2-yl)-4-methoxy-1H-indole-2-carboxamide (Cmpd No. 6). Samples from two, 50-mmol replicate syntheses were combined and were purified by silica gel chromatography using chloroform/methanol (0-4% methanol step gradient) to afford 29.9 mg (59% from resin 3) after overnight drying at 40° C. under house vacuum: ¹HNMR (methanol-d₄), d 1.59-1.70 (m, 2H), 2.02 (ddd, 1H, J=15.3, 11.3, and 4.6 Hz), 2.13 (dddd, 1H, J=15.1, 11.8, 9.5, and 2.3 Hz), 2.35 (ddd, 1H, J=14.1, 9.6, and 4.7 Hz), 2.99-3.19 (3m, 4H), 3.79 (s, 3H), 4.34 (dd, 1H, J=11.2 and 4.2 Hz), 4.76 (dd, 1H, J=8.9 and 6.0 Hz), 6.37 (d, 1H, J=7.7 Hz), 6.79 (t, 1H, J=8.5 Hz), 6.89 (d, 1H, J=8.2 Hz), 6.95-7.03 (2m, 3H), 7.10-7.15 (2m, 2H); ¹³CNMR (methanol-d₄) δ 27.4, 33.1, 36.7, 38.3, 40.1, 51.6, 54.3, 55.0, 98.9, 101.6, 104.8, 113.2 (d, ²J_{CF}=21.2 Hz), 115.7 (d, ²J_{CF}=21.6 Hz), 118.7, 124.9 (d, ⁴J_{CF}=2.7 Hz), 125.0, 128.7, 129.8 (d, ³J_{CF}=8.5 Hz), 138.4, 140.0 (d, ³J_{CF}=7.4 Hz), 154.2, 162.6, 162.8 (d, ¹J_{CF}=244 Hz), 172.5, 174.9, 180.6.

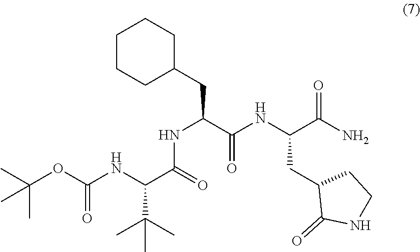

tert-Butyl ((S)-1-(((S)-1-(((S)-1-amino-1-oxo-3-((S)-2-oxopyrrolidin-3-yl)propan-2-yl)amino)-3-cyclohexyl-1-oxopropan-2-yl)amino)-3,3-dimethyl-1-oxobutan-2-yl)carbamate (Cmpd No. 7). The crude material was dissolved in 500 mL of pure methanol and dried with nitrogen followed by ˜1 mL of chloroform and was dispensed onto a 500-mg HyperSep SI cartridge of silica gel which had been equilibrated with chloroform. Elution with chloroform/methanol (0-4% methanol) in step gradient manner afforded 12.7 mg (29.5%) of 7; ¹HNMR (methanol-d₄), d 0.80-1.97 (m, 33H), 2.19 (ddd, 1H, J=15.6, 11.5, and 4.3 Hz), 2.33 (dddd, 1H, J=15.2 Hz, J=11.1, 8.9, and 2.5 Hz), 2.53 (ddd, 1H, J=13.1, 9.9 and 3.9 Hz), 3.25-3.35 (m, 2H), 3.94 (s, 1H), 4.40-4.50 (2m, 2H); ¹³CNMR (methanol-d₄), d 25.8, 25.9, 26.0, 26.2, 27.3, 27.4, 32.1, 33.4, 33.4, 33.86, 33.88, 38.2, 38.7, 40.0, 51.1, 51.3, 62.3, 79.2, 156.4, 172.1, 173.4, 174.9, 180.5.

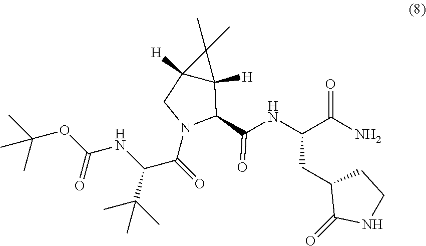 [SMILES: CC(C)(C)OC(=O)N[C@H](C(=O)N1C[C@H]2[C@@H]([C@H]1C(=O)N[C@@H](C[C@@H]1CCNC1=O)C(N)=O)C2(C)C)C(C)(C)C]

tert-Butyl ((S)-1-((1R,2S,5S)-2-(((S)-1-amino-1-oxo-3-((S)-2-oxopyrrolidin-3-yl)propan-2-yl)carbamoyl)-6,6-dimethyl-3-azabicyclo[3.1.0]hexan-3-yl)-3,3-dimethyl-1-oxobutan-2-yl)carbamate, (Cmpd No. 8). Samples from two, 50-mmol replicate syntheses were combined and were purified by silica gel chromatography using chloroform/methanol (0-3% methanol step gradient) to afford 29.2 mg (56% from resin 3) after overnight drying at 40° C. under house vacuum: Samples from two, 50-mmol replicate syntheses were combined and were purified by silica gel chromatography using chloroform/methanol (0-3% methanol step gradient) to afford 29.2 mg (56% from resin 3) after overnight drying at 40° C. under house vacuum: ¹HNMR (methanol-d₄), rotamer ratio indeterminate, line list given for the dominant rotamer, d 0.97 (s, 3H), 1.02 (s, 9H), 1.09 (s, 3H), 1.43 (s, 9H), 1.53 (d, 1H, J=7.7 Hz), 1.59 (dd, 1H, J=7.3 and 4.2 Hz), 1.74-1.90 (m, 2H), 2.17 (ddd, 1H, 13.8, 12.2, and 4.3 Hz), 2.35 (m, 1H), 2.67 (ddd, 1H, J=14.0, 10.0, and 4.3 Hz), 3.26-3.35 (m, 2H), 3.97-4.04 (m, 2H), 4.22 (d, 1H, J=9.7 Hz), 4.35 (s, 1H), 4.48 (dd, 1H, J=11.8 and 3.8 Hz), 6.39 (br d, 1H, J=9.7 Hz, NH); ¹³CNMR (methanol-d₄), line list given for the dominant rotamer, d 11.7, 19.0, 25.1, 25.6, 27.2, 27.4, 27.7, 30.8, 33.5, 34.4, 38.1, 40.1, 51.2, 58.8, 58.9, 60.8, 79.2, 156.7, 171.3, 172.6, 175.1, 180.7.

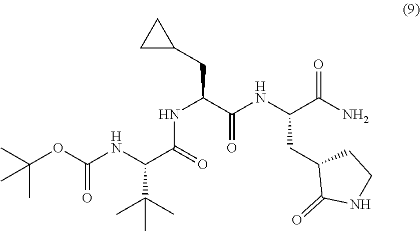 [SMILES: CC(C)(C)OC(=O)N[C@H](C(=O)N[C@@H](CC1CC1)C(=O)N[C@@H](C[C@@H]1CCNC1=O)C(N)=O)C(C)(C)C]

tert-Butyl ((S)-1-(((S)-1-(((S)-1-amino-1-oxo-3-((S)-2-oxopyrrolidin-3-yl)propan-2-yl)amino)-3-cyclopropyl-1-oxopropan-2-yl)amino)-3,3-dimethyl-1-oxobutan-2-yl)carbamate (Cmpd No. 9). Samples from two, 50-mmol replicate syntheses were combined and were triturated under 4-5 mL of boiling chloroform. The solution was allowed to cool to room temperature and was decanted with a Pasteur pipet onto a 500-mg HyperSep SI cartridge of silica gel equilibrated with chloroform leaving behind 24.1 mg of a gelatinous material. The column was eluted with chloroform/methanol mobile phases (0-5% methanol) in step gradient manner to afford 8.5 mg (17% from resin 3) of 9; ¹HNMR (methanol-d₄), d −0.054-0.046 (m, 2H), 0.27-0.38 (m, 2H), 0.61-0.70 (m, 1H), 0.85 (s, 9H), 1.31 (s, 9H), 1.41-1.48 (m, 1H), 1.53-1.63 (m, 2H), 1.65-1.75 (m, 1H), 2.04 (ddd, 1H, J=14.1, 11.5, and 4.1 Hz), 2.19 (ddd, 1H, J=15.2, 11.0, 8.8, and 2.5 Hz), 2.43 (ddd, 1H, J=13.2, 9.9, 3.9 Hz), 3.10-3.21 (m, 2H), 3.80 (s, 1H), 4.23 (t, 1H, J=7.3 Hz), 4.34 (dd, 1H, J=11.4 and 4.0 Hz).

The gelatinous material from above was dissolved in 100 mL of methanol and was dispensed onto a dry 500-mg HyperSep SI cartridge of silica gel. The column was dried using a slow, reverse-flow of dry nitrogen gas. The column was then eluted with chloroform/methanol mobile phases (0-5% methanol) in step gradient manner to afford 16.1 mg (32% from resin 3) of 9; the proton NMR spectrum was identical to the sample of 9 described above;); ¹³CNMR (methanol-d₄) δ 1.28, 1.42, 4.88, 23.5, 25.06, 25.15, 31.3, 31.7, 34.0, 35.9, 37.8, 48.8, 52.0, 59.9, 76.9, 154.1, 169.7, 170.6, 172.6, 178.2.

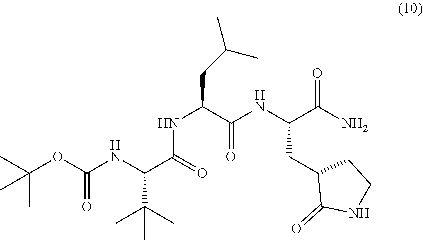 [SMILES: CC(C)C[C@H](NC(=O)[C@@H](NC(=O)OC(C)(C)C)C(C)(C)C)C(=O)N[C@@H](C[C@@H]1CCNC1=O)C(N)=O]

tert-Butyl ((S)-1-(((S)-1-(((S)-1-amino-1-oxo-3-((S)-2-oxopyrrolidin-3-yl)propan-2-yl)amino)-4-methyl-1-oxopentan-2-yl)amino)-3,3-dimethyl-1-oxobutan-2-yl)carbamate (Cmpd No. 10). Samples from two, 50-mmol replicate syntheses were combined in 100 mL of methanol. To the solution was added approximately 100 mg of silica gel and the mixture was dried to a powder and was then poured onto a dry, 500-mg HyperSep SI cartridge of silica gel. The column was eluted with chloroform/methanol mobile phases (0-4% methanol) in step gradient manner to afford 22.7 mg (17% from resin 3) of 10 after overnight drying at 40° C. under house vacuum: ¹HNMR (methanol-d₄), d 0.94 (d, 3H, J=6.4 Hz), 0.99 (d, 3H), 1.00 (s, 9H), 1.46 (s, 9H), 1.60-1.65 (m, 2H), 1.68-1.90 (m, 3H), 2.19 (ddd, 1H, J=14.3, 11.5, and 4.3 Hz), 2.33 (dddd, 1H, J=15.2, 11.1, 8.8, 2.5 Hz), 2.55 (ddd, 1H, J=13.5, 9.9, 4.1 Hz), 3.26-3.36 (m, 2H), 3.95 (s, 1H), 4.42 (t, 1H, J=7.6 Hz), 4.47 (dd, 1H, J=11.4 and 4.1 Hz); ¹³CNMR (methanol-d₄) δ 20.7, 22.0, 24.4, 25.8, 27.3, 27.4, 33.4, 33.9, 38.2, 40.0, 40.2, 51.1, 52.1, 62.2, 79.2, 156.4, 172.1, 173.3, 174.9, 180.5.

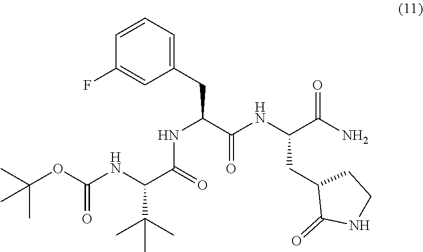 [SMILES: CC(C)(C)OC(=O)N[C@H](C(=O)N[C@@H](Cc1cccc(F)c1)C(=O)N[C@@H](C[C@@H]1CCNC1=O)C(N)=O)C(C)(C)C]

tert-Butyl ((S)-1-(((S)-1-(((S)-1-amino-1-oxo-3-((S)-2-oxopyrrolidin-3-yl)propan-2-yl)amino)-3-(3-fluorophenyl)-1-oxopropan-2-yl)amino)-3,3-dimethyl-1-oxobutan-2-yl)carbamate (Cmpd No. 11). Samples from two, 50-mmol replicate syntheses were combined and the crude material was dissolved in approximately 2 mL of boiling 97/3 chloroform/methanol, was allowed to cool and was dispensed onto a 500-mg HyperSep SI cartridge of silica gel which had been equilibrated in chloroform. The column was eluted with chloroform/methanol (0-5% methanol) in a step gradient manner to give 34.8 mg (63% from resin 3) of 11 after drying overnight under house vacuum at 40° C.; ¹HNMR (methanol-d₄), d 0.92 (s, 9H), 1.33 (s, 9H), 1.60-1.74 (m, 2H), 2.00 (ddd, 1H, J=14.1, 11.6, and 4.1 Hz), 2.18 (dddd, 1H, J=15.2, 11.0, 8.9, and 2.5 Hz), 2.36 (ddd, 1H, J=13.2, 10.1, and 4.1 Hz), 2.89 (dd, 1H, J=13.8 and 8.2 Hz), 3.02 (dd, 1H, J=13.8 and 7.0 Hz), 3.12-3.23 (m, 2H), 3.80 (s, 1H), 4.27 (dd, 1H, J=11.5 and 4.0 Hz), 4.53 (t, 1H, J=7.5 Hz), 6.84 (dt, 1H, j=8.5 and 2.1 Hz), 6.94 (d, 1H, J=9.9 Hz), 6.98 (d, 1H, J=7.7 Hz), 7.17 (q, 1H, J=7.3 Hz); ¹³CNMR (methanol-d₄) δ 25.7, 27.3, 33.2, 34.0, 36.8, 38.0, 40.0, 51.2, 54.6, 62.2, 79.2, 113.3 (d, ²J_{CF}=21.2 Hz), 115.8 (d, ²J_{CF}=21.6 Hz), 124.9 (d, ⁴J_{CF}=2.3 Hz), 129.8 (d, ³J_{CF}=8.2 Hz), 139.6 (d, ³J_{CF}=7.6 Hz), 156.3, 162.8 (d, ¹J_{CF}=245 Hz), 171.9 (two overlapping lines?), 174.7, 180.4.

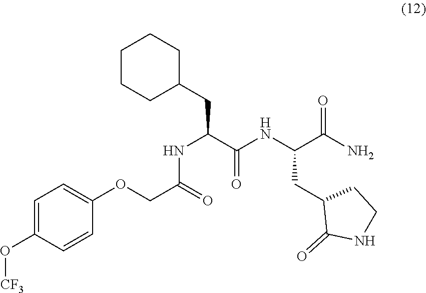 [SMILES: NC(=O)[C@H](C[C@@H]1CCNC1=O)NC(=O)[C@H](CC1CCCCC1)NC(=O)COc1ccc(OC(F)(F)F)cc1]

(S)—N—((S)-1-amino-1-oxo-3-((S)-2-oxopyrrolidin-3-yl)propan-2-yl)-3-cyclohexyl-2-(2-(4-(trifluoromethoxy)phenoxy)acetamido)propenamide (Cmpd No. 12). Samples from two, 50-mmol replicate syntheses were combined in 250 mL of chloroform and were chromatographed on a 500-mg HyperSep SI cartridge of silica gel which had been equilibrated in chloroform. Elution with chloroform/methanol mobile phases (0-3% methanol) in a step gradient manner gave 30.1 mg (55% from resin 3) of 12 after drying in vacuo overnight at 40° C.; ¹HNMR (methanol-d₄), d 0.87-1.01 (m, 2H), 1.11-1.34 (m, 4H), 1.60-1.88 (m, 9H), 2.14 (ddd, 1H, J=14.1, 11.5, and 4.7 Hz), 2.29 (dddd, 1H, J=15.2, 10.9, 9.0, and 2.4 Hz), 2.52 (ddd, 1H, J=14.1, 9.4, 4.8 Hz), 3.22-3.33 (m, 2H), 4.45 (dd, 1H, J=11.4 and 4.2 Hz), 4.50 (dd, 1H, J=99.9 and 5.3 Hz), 4.62 (d, 1H, J=15.0 Hz), 4.69 (d, 1H, J=15.0 Hz), 7.08 (d, 2H, J=9.2 Hz), 7.24 (d, 2H, J=8.8 Hz); ¹³CNMR (methanol-d₄) d 25.7, 25.9, 26.1, 27.5, 31.8, 33.2, 33.5, 33.9, 38.4, 38.7, 40.1, 115.6, 120.6 (q, ¹J_{CF}=255 Hz), 122.2, 143.3 (q, ³J_{CF}=2.0 Hz), 156.6, 169.8, 173.5, 175.0, 180.6.

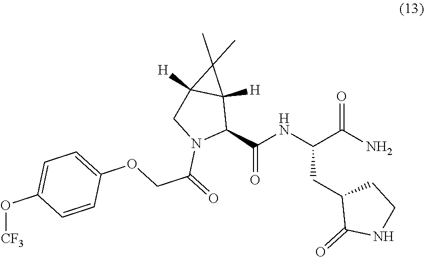 [SMILES: CC1(C)[C@@H]2[C@@H](C(=O)N[C@@H](C[C@@H]3CCNC3=O)C(N)=O)N(C(=O)COc3ccc(OC(F)(F)F)cc3)C[C@@H]21]

(1R,2S,5S)—N—((S)-1-Amino-1-oxo-3-((S)-2-oxopyrrolidin-3-yl)propan-2-yl)-6,6-dimethyl-3-(2-(4-(trifluoromethoxy)phenoxy)acetyl)-3-azabicyclo[3.1.0]hexane-2-carboxamide (Cmpd No. 13). Purified by silica gel chromatography using chloroform/methanol (0-4% methanol step gradient) to afford 16.7 mg (63% from resin 3) after overnight drying at 40° C. under house vacuum: ¹HNMR (methanol-d₄), rotamer ratio 85:15, line list given for the dominant rotamer, d 1.01 (s, 3H), 1.12 (s, 3H), 1.58 (d, 1H, J=7.6 Hz), 1.64 (dd, 1H, J=7.4 and 5.4 Hz), 1.77-1.86 (m, 2H), 2.08-2.15 (m, 1H), 2.21-2.28 (m, 1H), 2.60 (ddd, 1H, J=14.4, 9.4, and 5.1 Hz), 3.14 (ddd, 1H, J=16.7, 9.5, and 7.2 Hz), 3.23 (dt, 1H, J=10.8, 9.2, and 1.8 Hz), 3.65 (d, 1H, J=10.5 Hz), 4.00 (dd, 1H, J=10.4 and 5.4 Hz), 4.37 (s, 1H), 4.43 (dd, 1H, J 12.0 and 4.0 Hz), 4.78 (d, 1H, J=15.5 Hz), 4.83 (d, 1H, J=15.6 Hz), 7.01 (d, 2H, J=9.1 Hz), 7.19 (d, 2H, J=8.7 Hz); ¹³CNMR (methanol-d₄), line list given for the dominant rotamer, d 11.6, 19.1, 24.9, 27.6, 27.7, 30.6, 33.1, 38.3, 40.1, 45.9, 51.7, 61.2, 66.1, 115.5, 120.6 (q, ¹J_{CF}=254 Hz), 122.0, 143.0, 156.9, 167.6, 172.5, 175.2, 180.9.

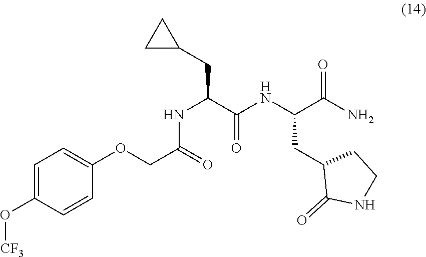 [SMILES: NC(=O)[C@H](C[C@@H]1CCNC1=O)NC(=O)[C@H](CC1CC1)NC(=O)COc1ccc(OC(F)(F)F)cc1]

(S)—N—((S)-1-amino-1-oxo-3-((S)-2-oxopyrrolidin-3-yl)propan-2-yl)-3-cyclopropyl-2-(2-(4-(trifluoro-methoxy)phenoxy)acetamido)propenamide (Cmpd No. 14). Samples from two, 50-mmol replicate syntheses were combined and were recrystallized from methanol to give 17.0 mg (34%) of S23-14; ¹HNMR (methanol-d₄), d 0.10-0.18 (m, 2H), 0.42-0.51 (m, 2H), 0.70-0.78 (m, 1H), 1.64-1.69 (m, 1H), 1.74-1.88 (m, 3H), 2.14 (dd, 1H, J=8.6, and 4.7 Hz), 2.27-2.32 (m, 1H, J=1H), 2.50-2.57 (m, 1H), 3.22-3.31 (m, 2H), 4.44 (2m, 2H), 4.62 (d, 1H, J=14.8 Hz), 4.67 (d, 1H, J=14.9 Hz), 7.09 (d, 2H, J=8.0 Hz), 7.24 (d, 2H, J=8.5 Hz); ¹³CNMR (methanol-d₄) δ 1.42, 1.92, 5.23, 25.7, 31.5, 34.4, 36.5, 38.3, 49.7, 52.3, 65.2, 113.8, 118.8 (q, ¹J_{CF}=255 Hz), 120.4, 141.5, 154.7, 167.7, 171.0, 173.1, 178.8.

The filtrate from the above recrystallization was evaporated to dryness and the residue was recrystallized from methanol to give 4.2 mg (8.3% from resin 3) of 14. Its proton NMR spectrum was identical to that of 14 obtained from the first recrystallization.

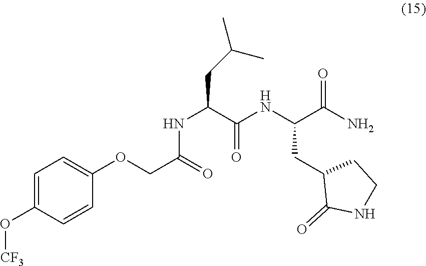 [SMILES: CC(C)C[C@H](NC(=O)COc1ccc(OC(F)(F)F)cc1)C(=O)N[C@@H](C[C@@H]1CCNC1=O)C(N)=O]

(S)—N—((S)-1-Amino-1-oxo-3-((S)-2-oxopyrrolidin-3-yl)propan-2-yl)-4-methyl-2-(2-(4-(trifluormeth-oxy)phenoxy)acetamido)pentanamide (15). Samples from two, 50-mmol replicate syntheses were combined and were triturated under 4-5 mL of boiling chloroform. The solution was allowed to cool to room temperature and was dispensed onto a 500-mg HyperSep SI cartridge of silica gel which had been equilibrated with chloroform. The column was eluted with chloroform/methanol mobile phases (0-4% methanol) in step gradient manner to afford 24.4 mg (40% from 3) of 15 contaminated with approximately 17% by weight of the un-N-capped dipeptide. A fraction of 15 was isolated (3.1 mg, 6% from 3), which was found to be 90-95% pure by weight by proton NMR analysis: ¹HNMR (methanol-d₄), d 0.91 (d, 3H, J=6.3 Hz), 0.95 (d, 3H, J=6.4 Hz), 1.55-1.63 (m, 1H), 1.63-1.71 (m, 2H), 1.77-1.87 (m, 2H), 2.14 (ddd, 1H, J=14.1, 13.5, and 4.8 Hz), 2.29 (dddd, 1H, J=15.5, 11.2, 8.7, and 2.7 Hz), 2.52 (ddd, 1H, J=14.4, 9.6, and 4.9 Hz), 3.22-3.35 (m, 2H), 4.43 (dd, 1H, J=11.5 and 4.2 Hz), 4.46 (dd, 1H, J=8.9 and 6.0 Hz), 4.62 (d, 1H, J=14.9 Hz), 4.69 (d, 1H, J=14.9 Hz), 7.08 (d, 2H, J=9.1 Hz), 7.24 (d, 2H, J=8.6 Hz); ¹³CNMR (methanol-d₄) δ 20.3, 22.0, 24.5, 27.5, 33.2, 38.4, 40.0, 40.1, 51.6, 51.9, 67.0, 115.6, 120.6 (q, ¹J_{CF}=255 Hz), 122.2, 143.3, 156.5, 169.8, 173.4, 175.0, 180.7.

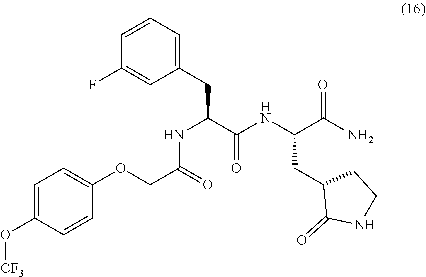 [SMILES: NC(=O)[C@H](C[C@@H]1CCNC1=O)NC(=O)[C@H](Cc1cccc(F)c1)NC(=O)COc1ccc(OC(F)(F)F)cc1]

(S)—N—((S)-1-amino-1-oxo-3-((S)-2-oxopyrrolidin-3-yl)propan-2-yl)-3-(3-fluorophenyl)-2-(2-(4-(trifluoromethoxy)phenoxy)acetamido)propenamide (Cmpd No. 16). Samples from two, 50-mmol replicate syntheses were combined in 200 mL and the solution was dispensed onto a dry 500-mg HyperSep SI cartridge of silica gel. The column was then dried with a slow stream of nitrogen gas in reverse direction. The column was eluted using chloroform/methanol (0-5% methanol step gradient) to afford 14.1 mg (25% from resin 3) after overnight drying at 40° C. under house vacuum: ¹HNMR (methanol-d₄), 1.63-1.78 (m, 2H), 1.95-2.05 (m, 1H), 2.12-2.23 (m, 1H), 2.29-2.40 (m, 1H), 2.90-2.97 (m, 1H), 3.09-3.20 (m, 3H), 4.28-4.34 (m, 1H), 4.40 (d, 1H, J=15.0 Hz), 4.48 (d, 1H, J=15.0 Hz), 4.58-4.65 (m, 1H), 6.79-6.88 (m, 3H), 6.88-7.00 (m, 2H), 7.03-7.11 (m, 2H), 7.11-7.20 (m, 1H); ¹³CNMR (methanol-d₄) δ 27.5, 33.2, 36.6, 38.3, 40.1, 51.7, 54.3, 67.0, 113.3 (d, ²J_{CF}=21.3 Hz), 115.5, 115.7 (d, ²J_{CF}=21.6 Hz), 120.6 (q, ¹J_{CF}=255 Hz), 122.2, 124.9 (d, ⁴J_{CF}=1.9 Hz), 129.8 (d, ³J_{CF}=8.5 Hz), 139.5 (d, ³J_{CF}=7.4 Hz), 143.3, 156.5, 162.8 (d, ¹J_{CF}=244 Hz), 169.6, 171.8, 174.9, 180.6.

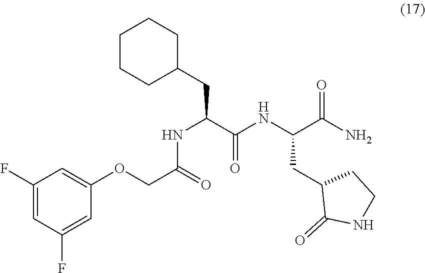 [SMILES: NC(=O)[C@H](C[C@@H]1CCNC1=O)NC(=O)[C@H](CC1CCCCC1)NC(=O)COc1cc(F)cc(F)c1]

(S)—N—((S)-1-amino-1-oxo-3-((S)-2-oxopyrrolidin-3-yl)propan-2-yl)-3-cyclohexyl-2-(3-(3,5-difluorophenyl)propanamido)propenamide (Cmpd No. 17). Purified by silica gel chromatography using chloroform/methanol (0-5% methanol step gradient) to afford 15.3 mg (62% from resin 3) after overnight drying at 40° C. under house vacuum: ¹HNMR (methanol-d₄), 0.89-1.01 (m, 2H), 1.14-1.33 (m, 4H), 1.49-1.64 (m, 2H), 1.64-1.76 (m, 5H), 1.78-1.90 (m, 2H), 2.12 (ddd, 1H, J=14.0, 11.4, and 4.8 Hz), 2.29-2.37 (m, 1H), 2.52 (ddd, 1H, J=14.2, 9.3, and 4.9 Hz), 2.61 (t, 2H, J=7.3 Hz), 2.96 (t, 2H, J=7.4 Hz), 3.33-3.37 (m, 2H), 4.32 (dd, 1H, J=10.1 and 5.0 Hz), 4.41 (dd, 1H, J=11.4 and 4.2 Hz), 6.77 (tt, 1H, J=9.2 and 2.2 Hz), 6.86 (d, 2H, j=6.6 Hz); ¹³CNMR (methanol-d₄) δ 25.7, 25.9, 26.1, 30.7, 31.8, 33.1, 33.5, 33.8, 36.0, 38.4, 38.7, 40.2, 51.58, 51.62, 100.9 (t, ²J_{CF}=25.7 Hz), 110.9 (dd, ²J_{CF}=18.4 Hz and ⁴J_{CF}=6.6 Hz), 145.4 (t, ³J_{CF}=9.1 Hz), 163.1 (dd, ¹J_{CF}=245 Hz and ³J_{CF}=12.9 Hz), 173.6, 174.0, 175.1, 180.7.

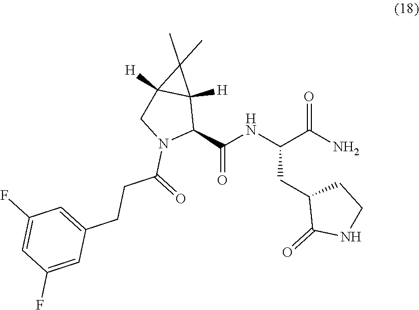 [SMILES: CC1(C)[C@@H]2[C@@H](C(=O)N[C@@H](C[C@@H]3CCNC3=O)C(N)=O)N(C(=O)CCc3cc(F)cc(F)c3)C[C@@H]21]

(1R,2S,5S)—N—((S)-1-Amino-1-oxo-3-((S)-2-oxopyrrolidin-3-yl)propan-2-yl)-3-(3-(3,5-difluorophenyl)-propanoyl)-6,6-dimethyl-3-azabicyclo[3.1.0]hexane-2-carboxamide (Cmpd No. 18). Samples from two, 50-mmol replicate syntheses were combined and were purified by silica gel chromatography using chloroform/methanol (0-3.5% methanol step gradient) to afford 22.4 mg (47% from resin 3) of 18 after overnight drying at 40° C. under house vacuum: ¹HNMR (methanol-d₄), rotamer ratio 83:17, line list given for the dominant rotamer, d 0.87 (s, 3H), 1.05 (s, 3H), 1.48-1.53 (m, 2H), 1.80-1.86 (m, 2H), 2.05 (dd, 1H, J=14.0, 11.9, and 5.3 Hz), 2.28-2.37 (m, 1H), 2.53-2.61 (m, 1H), 2.62-2.72 (m, 2H), 2.90 (t, 2H, J=7.2 Hz), 3.29-3.32 (m, 2H), 3.50 (d, 1H, J=10.5 Hz), 3.93 (dd, 1H, J=10.6 and 5.1 Hz), 4.24 (s, 1H), 4.36 (dd, 1H, J=11.8 and 4.0 Hz), 6.72 (tt, 1H, J=9.2 and 2.5 Hz), 6.84-6.85 (m, 2H); ¹³CNMR (methanol-d₄), line list given for the dominant rotamer, d 11.4, 18.9, 24.9, 27.3, 27.7, 29.7, 31.0, 32.9, 34.7, 38.5, 40.2, 52.0, 61.0, 100.8 (t, ²J_{CF}=25.9 Hz), 111.0 (dd, ²J_{CF}=24.9 Hz and ⁴J_{CF}=6.5 Hz), 145.6 (t, ³J_{CF}=9.1 Hz), 163.1 (dd, ¹J_{CF}=246 Hz and ³J_{CF}=13.1 Hz), 171.7, 172.9, 175.3, 180.9.

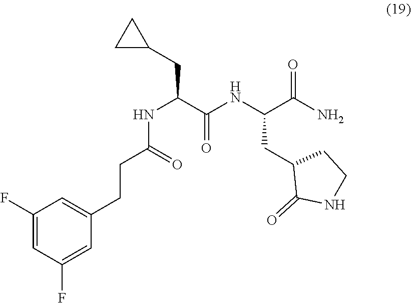 [SMILES: NC(=O)[C@H](C[C@@H]1CCNC1=O)NC(=O)[C@H](CC1CC1)NC(=O)CCc1cc(F)cc(F)c1]

(S)—N—((S)-1-amino-1-oxo-3-((S)-2-oxopyrrolidin-3-yl)propan-2-yl)-3-cyclopropyl-2-(3-(3,5-difluorophenyl)propanamido)propenamide (Cmpd No. 19). Samples from two, 50-mmol replicate syntheses were combined and were triturated under 4 mL of boiling chloroform. After cooling, the solid was collected by suction filtration to give 21.7 mg (48% from resin 3) of 19 after overnight drying at 40° C. under house vacuum: ¹HNMR (methanol-d₄), d −0.057-0.054 (m, 2H), 0.28-0.39 (m, 2H), 0.55-0.64 (m, 1H), 1.38-1.45 (m, 1H), 1.52-1.59 (m, 1H), 1.65-1.78 (m, 2H), 2.01 (ddd, 1H, J=14.6, 11.5, and 4.8 Hz), 2.22 (dddd, 1H, J=15.1, 11.6, 9.0, 2.8 Hz), 2.38-2.45 (m, 1H), 2.45-2.55 (m, 2H), 2.84 (t, 2H, J=7.4 Hz), 3.16-3.25 (m, 2H), 4.20 (dd, 1H, J=8.2 and 5.9 Hz), 4.31 (dd, 1H, J=11.3 and 4.2 Hz), 6.64 (t, 1H, J=9.2 Hz), 6.74 (d, 2H, J=6.8 Hz);); ¹³CNMR (methanol-d₄) δ 1.42, 1.97, 5.28, 25.7, 28.9, 31.3, 34.3, 34.4, 36.5, 38.3, 49.7, 52.7, 99.0 (t, ²J_{CF}=25.8 Hz), 109.0 (dd, ²J_{CF}=17.6 Hz and ⁴J_{CF}=6.5 Hz), 143.5 (t, ³J_{CF}=9.2 Hz), 161.2 (dd, ¹J_{CF}=247 Hz and ³J_{CF}=13.1 Hz), 171.4, 171.6, 173.1, 178.8.

The filtrate from the above-described trituration was dispensed onto a 500-mg column of silica gel equilibrated with chloroform. Elution with chloroform/methanol mobile phases (0-10% methanol) in step gradient manner gave 3.4 mg (7.5% from 3) of 19.

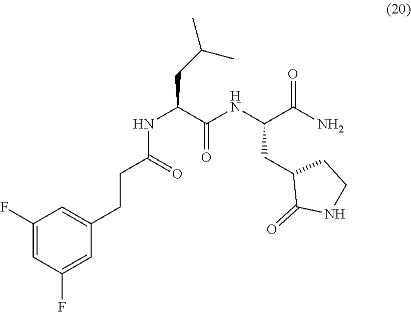 [SMILES: CC(C)C[C@H](NC(=O)CCc1cc(F)cc(F)c1)C(=O)N[C@@H](C[C@@H]1CCNC1=O)C(N)=O]

(S)—N—((S)-1-Amino-1-oxo-3-((S)-2-oxopyrrolidin-3-yl)propan-2-yl)-2-(3-(3,5-difluorophenyl)propan-amido)-4-methylpentanamide (Cmpd No. 20). Samples from two, 50-mmol replicate syntheses were combined and were triturated under 4-5 mL of boiling chloroform. The solution was allowed to cool to room temperature and was filtered to afford 13.4 mg (30% from resin 3) of 20; ¹HNMR (methanol-d₄), d 0.88 (d, 3H, J=6.1 Hz), (d, 3H, J=6.2 Hz), 1.47-1.58 (m, 3H), 1.78-1.90 (m, 2H), 2.12 (ddd, 1H, J=14.1, 11.5, and 4.9 Hz), 2.33 (dddd, 1H, J=15.4, 11.9, 9.2, 3.1 Hz), 2.51 (ddd, 1H, J=14.2, 9.4, 5.0 Hz), 2.55-2.67 (m, 2H), 2.96 (t, 2H, J=7.4 Hz), 3.28-3.37 (m, 2H), 4.31 (dd, 1H, J=8.9 and 6.6 Hz), 4.40 (dd, 1H, J=11.3 and 4.2 Hz), 6.77 (tt, 1H, J=9.2 and 2.2 Hz), 6.83-6.89 (m, 2H); ¹³CNMR (methanol-d₄) δ 20.2, 22.1, 24.4, 27.5, 30.8, 33.1, 36.2, 38.4, 40.1, 40.2, 51.6, 52.2, 100.9 (t, ²J_{CF}=25.9 Hz), 111.0 (dd, ²J_{CF}=17.8 Hz and ⁴J_{CF}=6.5 Hz), 145.3 (t, ³J_{CF}=9.1 Hz), 163.1 (dd, ¹J_{CF}=247 Hz and ³J_{CF}=13.1 Hz), 173.6, 173.9, 175.1, 180.7. The filtrate was dispensed onto a 500-mg HyperSep SI cartridge of silica gel, which had been equilibrated in chloroform. Elution with chloroform/methanol mobile phases (0-5% methanol) in step gradient manner afforded 12.4 mg (27% from resin 3) of 20 whose proton NMR spectrum was identical to the triturated material described above.

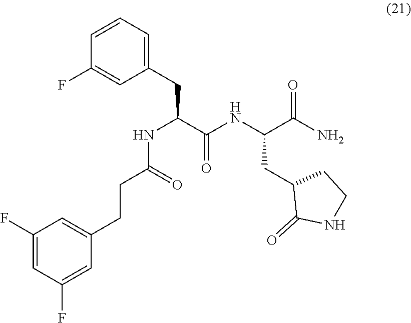 [SMILES: NC(=O)[C@H](C[C@@H]1CCNC1=O)NC(=O)[C@H](Cc1cccc(F)c1)NC(=O)CCc1cc(F)cc(F)c1]

(S)—N—((S)-1-amino-1-oxo-3-((S)-2-oxopyrrolidin-3-yl)propan-2-yl)-2-(3-(3,5-difluorophenyl)-propanamido)-3-(3-fluorophenyl)propenamide (Cmpd No. 21). Samples from two, 50-mmol replicate syntheses were combined, were dissolved in 150 mL of methanol, and the solution was dispensed onto a dry 500-mg HyperSep SI cartridge of silica gel. The column was dried under a slow, reverse flow of nitrogen gas overnight. The column was then eluted with chloroform/methanol mobile phases (0-4% methanol) in step gradient manner to afford 25.0 mg (49% from 3) of 21 after overnight drying at 40° C. under house vacuum: ¹HNMR (methanol-d₄), d 1.76-1.87 (m, 2H), 2.09 (ddd, 1H, J=14.2, 11.5, and 4.8 Hz), 2.30 (dddd, 1H, J=1H, J=15.2, 11.5, 8.9, and 2.8 Hz), 2.40-2.47 (m, 1H), 2.48-2.59 (m, 2H), 2.85 (t, 2H, J=7.5 Hz), 2.93 (dd, 1H, J=14.0 and 9.1 Hz), 3.15 (dd, 1H, J=14.0 and 5.5 Hz), 3.26-3.36 (m, 2H), 4.38 (dd, 1H, J=11.4 and 4.2 Hz), 4.60 (dd, 1H, J=9.0 and 5.5 Hz), 6.71-6.80 (m, 3H), 6.93-7.05 (m, 3H), 7.28 (q, 1H, J=7.3 Hz); ¹³CNMR (methanol-d₄) d 27.5, 30.7, 33.1, 36.1, 36.7, 38.3, 40.1, 51.7, 54.8, 100.9 (t, ²J_{CF}=25.7 Hz), 110.8 (dd, ²J_{CF}=18.4 Hz and ⁴J_{CF}=6.5 Hz), 113.2 (d, ²J_{CF}=21.2 Hz), 115.7 (d, ²J_{CF}=21.4 Hz), 124.8 (d, ⁴J_{CF}=2.7 Hz), 129.8 (d, ³J_{CF}=8.2 Hz), 139.8 (d, ³J_{CF}=7.4 Hz), 145.3 (d, ³J_{CF}=9.1 Hz), 162.8 (d, ¹J_{CF}=244 Hz), 163.0 (dd, ¹J_{CF}=247 Hz and ³J_{CF}=13.1 Hz), 172.3, 173.3, 174.9, 180.6.

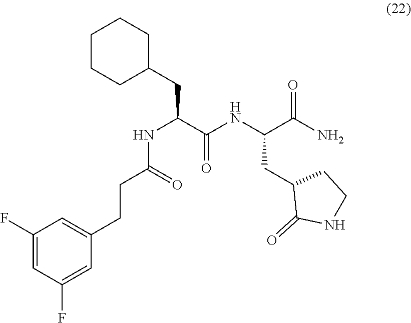 [SMILES: NC(=O)[C@H](C[C@@H]1CCNC1=O)NC(=O)[C@H](CC1CCCCC1)NC(=O)CCc1cc(F)cc(F)c1]

(S)—N—((S)-1-amino-1-oxo-3-((S)-2-oxopyrrolidin-3-yl)propan-2-yl)-3-cyclohexyl-2-(2-(4-fluorophenoxy)acetamido)propenamide (Cmpd No. 22). Samples from two, 50-mmol replicate syntheses were combined and were purified by silica gel chromatography using chloroform/methanol (0-4% methanol step gradient) to afford 16.3 mg (34% from resin 3) after overnight drying at 40° C. under house vacuum: ¹HNMR (methanol-d₄), d 0.86-1.02 (m, 2H), 1.15-1.31 (m, 4H), 1.59-1.89 (2m, 9H), 2.14 (ddd, 1H, J=14.1, 11.4, and 4.7 Hz), 2.30 (dddd, 1H, J=15.4, 11.3, 9.0, and 2.6 Hz), 2.51 (ddd, 1H, J=14.1, 9.5, and 4.8 Hz), 3.23-3.32 (m, 2H), 4.44 (dd, 1H, J=11.4 and 4.2 Hz), 4.49 (dd, 1H, J=9.8 and 5.2 Hz), 4.57 (d, 1H, J=15.0 Hz), 4.64 (d, 1H, J=15.0 Hz), 6.98-7.08 (m, 4H); ¹³CNMR (methanol-d₄) δ 25.7, 25.9, 26.1, 27.5, 31.8, 33.2, 33.5, 33.9, 38.4, 38.7, 40.1, 51.2, 51.5, 67.3, 115.5 (d, ²J_{CF}=23.5 Hz), 115.8 (d, ³J_{CF}=8.2 Hz), 154.0 (d, ⁴J_{CF}=2.1 Hz), 157.9 (d, ¹J_{CF}=238 Hz), 170.0, 173.5, 175.0, 180.6.

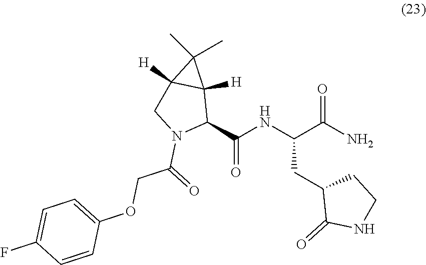 [SMILES: CC1(C)[C@@H]2[C@@H](C(=O)N[C@@H](C[C@@H]3CCNC3=O)C(N)=O)N(C(=O)COc3ccc(F)cc3)C[C@@H]21]

(1R,2S,5S)—N—((S)-1-Amino-1-oxo-3-((S)-2-oxopyrrolidin-3-yl)propan-2-yl)-3-(2-(4-fluorophenoxy)-acetyl)-6,6-dimethyl-3-azabicyclo[3.1.0]hexane-2-carboxamide (Cmpd No. 23). Samples from two, 50-mmol replicate syntheses were combined and were purified by silica gel chromatography using chloroform/methanol (0-3% methanol step gradient) to afford 26.2 mg (57% from resin 3) after overnight drying at 40° C. under house vacuum: ¹HNMR (methanol-d₄), rotamer ratio 83:17, line list given for the dominant rotamer, d 1.00 (s, 3H), 1.11 (s, 3H), 1.57 (d, 1H, J=7.5 Hz), 1.63 (dd, 1H, J=7.4 and 5.4 Hz), 1.76-1.87 (m, 2H), 2.08-2.16 (m, 1H), 2.24-2.31 (m, 1H), 2.60 (ddd, 1H, J=14.4, 9.4, and 5.1 Hz), 3.18 (ddd, 1H, J=16.8, 9.5, and 7.9 Hz), 3.25 (dt, 1H, J=10.9, 9.5, and 1.9 Hz), 3.65 (d, 1H, J=10.5 Hz), 3.99 (dd, 1H, J=10.5 and 5.4 Hz), 4.37 (s, 1H), 4.44 (dd, 1H, J 11.9 and 4.0 Hz), 4.72 (d, 1H, J=15.3 Hz), 4.77 (d, 1H, J=15.3 Hz), 6.90-6.97 (m, 2H), 6.97-7.03 (m, 2H); ¹³CNMR (methanol-d₄), line list given for the dominant rotamer, d 11.6, 19.1, 24.9, 27.6, 27.7, 30.6, 33.1, 38.3, 40.1, 45.9, 51.8, 61.2, 66.5, 115.3 (d, ²J_{CF}=23.3 Hz), 115.7 (d, ³J_{CF}=8.1 Hz), 154.4 (d, ⁴J_{CF}=2.1 Hz), 157.6 (d, ¹J_{CF}=238 Hz), 167.9, 172.5, 175.2, 180.9.

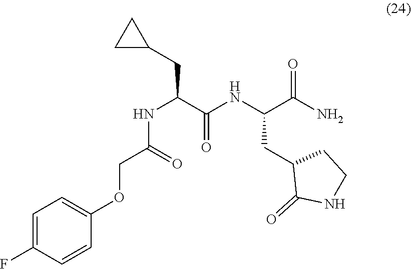 [SMILES: NC(=O)[C@H](C[C@@H]1CCNC1=O)NC(=O)[C@H](CC1CC1)NC(=O)COc1ccc(F)cc1]

(S)—N—((S)-1-amino-1-oxo-3-((S)-2-oxopyrrolidin-3-yl)propan-2-yl)-3-cyclopropyl-2-(2-(4-fluorophenoxy)-acetamido)propenamide (Cmpd No. 24). Samples from two, 50 mol replicate synthesis were combined using methanol, concentrated using nitrogen gas, and resolubilized in roughly 500 μL of 95/5 chloroform/methanol, blown down to a suitable volume, and dispensed onto a 500 mg HyperSep SI cartridge of silica gel. The material was then eluded with chloroform/methanol mobile phases (0-4.5% methanol) in step gradient manner to afford 17.1 mg (39% from 3) of 24 after overnight drying at 40° C. under house vacuum: ¹HNMR (methanol-d₄), d 0.05-0.18 (m, 2H), 0.38-0.50 (m, 2H), 0.66-0.78 (m, 1H), 1.60-1.88 (m, 4H), 2.06-2.17 (m, 1H), 2.23-2.33 (m, 1H), 2.45-2.55 (m, 1H), 3.19-3.29 (m, 2H), 4.40-4.50 (m, 2H), 4.56 (dd, 2H, J=14.9 and 20.3 Hz), 6.95-7.06 (m, 4H); ¹³CNMR (methanol-d₄), d 0.9, 1.4, 4.7, 25.1, 31.0, 33.9, 36.0, 37.8, 49.1, 51.7, 65.0, 113.1 (d, ²J_{CF}=23.5 Hz), 113.5 (d, ³J_{CF}=8.1 Hz), 151.7 (d, ⁴J_{CF}=2.1 Hz), 155.5 (d, ¹J_{CF}=238 Hz), 167.4, 170.4, 172.6, 178.3.

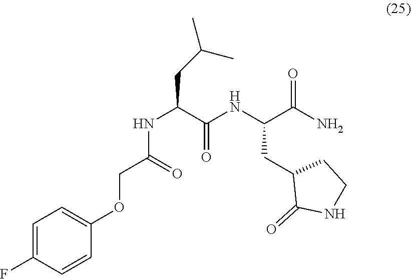 [SMILES: CC(C)C[C@H](NC(=O)COc1ccc(F)cc1)C(=O)N[C@@H](C[C@@H]1CCNC1=O)C(N)=O]

(S)—N—((S)-1-amino-1-oxo-3-((S)-2-oxopyrrolidin-3-yl)propan-2-yl)-2-(2-(4-fluorophenoxy)acetamido)-4-methylpentanamide (Cmpd No. 25). Samples from two, 50-mmol replicate syntheses were combined and were triturated under 6 mL of boiling chloroform. After cooling, the solid was collected by suction filtration to give 11.4 mg (26% from resin 3) of 25 after overnight drying at 40° C. under house vacuum: ¹HNMR (methanol-d₄), d 0.92 (d, 3H, J=6.1 Hz), 0.96 (d, 3H, J=6.3 Hz), 1.56-1.69 (2m, 3H), 1.76-1.89 (m, 2H), 2.14 (ddd, 1H, J=14.0, 11.3, and 4.7 Hz), 2.30 (dddd, 1H, J=15.3, 11.1, 8.6, and 2.7 Hz), 2.51 (ddd, 1H, J=14.2, 9.5, and 4.9 Hz), 3.23-3.33 (m, 2H), 4.44 (dd, 1H, J=11.4 and 4.2 Hz), 4.47 (t, 1H, J=6.9 Hz), 4.57 (d, 1H, J=14.9 Hz), 4.62 (d, 1H, J=15.1 Hz), 6.99-707 (m, 4H). The filtrate was dispensed onto a 500-mg HyperSep SI cartridge of silica gel which had been equilibrated in chloroform. The column was eluted with chloroform/methanol mobile phases (0-4% methanol) in a step gradient manner to afford 16.9 mg (39% from resin 3) of 25 after overnight drying at 40° C. under house vacuum. Its proton NMR spectrum was identical to the triturated sample above. ¹³CNMR (methanol-d₄) δ 20.4, 22.0, 24.5, 27.5, 33.2, 38.4, 40.12, 40.13, 51.5, 51.9, 67.3, 115.5 (d, ²J_{CF}=23.3 Hz), 115.8 (d, ³J_{CF}=8.2 Hz), 154.1 (d, ⁴J_{CF}=2.9 Hz), 157.8 (d, ¹J_{CF}=238 Hz), 170.0, 173.4, 175.0, 180.6.

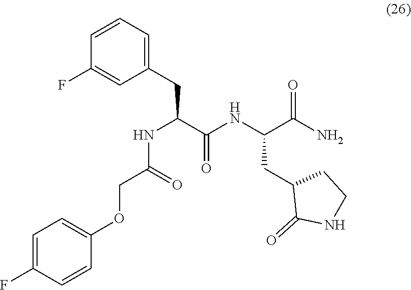 [SMILES: NC(=O)[C@H](C[C@@H]1CCNC1=O)NC(=O)[C@H](Cc1cccc(F)c1)NC(=O)COc1ccc(F)cc1]

(S)—N—((S)-1-Amino-1-oxo-3-((S)-2-oxopyrrolidin-3-yl)propan-2-yl)-2-(2-(4-fluorophenoxy)acetamido)-3-(3-fluorophenyl)propanamide (Cmpd No. 26). Purified by silica gel chromatography using chloroform/metha-nol (0-3% methanol step gradient) to afford 7.5 mg (31% from resin 3) after overnight drying at 40° C. under house vacuum: ¹HNMR (methanol-d₄), d 1.77-1.88 (m, 2H), 2.12 (ddd, 1H, J=14.1, 11.5, and 4.8 Hz), 2.30 (ddd, 1H, J=11.3, 8.9, 2.7 Hz), 2.45 (ddd, 1H, J=14.2, 11.5, 4.8 Hz), 3.05 (dd, 1H, J=14.0 and 8.9 Hz), 3.22-3.32 (m, 3H), 4.43 (dd, 1H, J=11.4 and 4.2 Hz), 4.46 (d, 1H, J=14.9 Hz), 4.54 (d, 1H, J=15.0 Hz), 4.73 (dd, 1H, J=8.9 and 5.4 Hz), 6.88-6.92 (m, 2H), 6.94-7.07 (m, 5H), 7.25-7.31 (m, 1H); ¹³CNMR (methanol-d₄) d 27.5, 33.2, 36.6, 38.3, 40.1, 51.7, 54.2, 67.3, 113.3 (d, ²J_{CF}=21.2 Hz), 115.5 (d, ²J_{CF}=23.5 Hz), 115.72 (d, ²J_{CF}=21.4 Hz), 115.75 (d, ³J_{CF}=8.2 Hz), 124.9 (d, ⁴J_{CF}=2.8 Hz), 129.9 (d, ³J_{CF}=8.3 Hz), 139.5 (d, ³J_{CF}=7.5 Hz), 154.0 (d, ⁴J_{CF}=2.1 Hz), 157.8 (d, ¹J_{CF}=238 Hz), 162.8 (d, ¹J_{CF}=244 Hz), 169.8, 171.8, 174.8, 180.6.

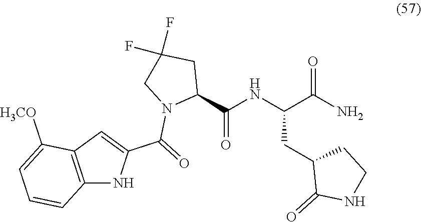 [SMILES: COc1cccc2[nH]c(C(=O)N3CC(F)(F)C[C@H]3C(=O)N[C@@H](C[C@@H]3CCNC3=O)C(N)=O)cc12]

(S)—N—((S)-1-amino-1-oxo-3-((S)-2-oxopyrrolidin-3-yl)propan-2-yl)-4,4-difluoro-1-(4-methoxy-1H-indole-2-carbonyl)pyrrolidine-2-carboxamide (Cmpd No. 57). Purified by silica gel chromatography using chloroform/methanol (0-4% methanol step gradient) to afford 14.4 mg (60% from resin 3) after overnight drying at 40° C. under house vacuum: ¹HNMR (methanol-d₄), d 1.34-2.03 (br m, 2H), 2.13 (dd, 1H, J=12.5 and 4.5 Hz), 2.20-2.75 (br m, 3H), 2.75-3.18 (br m, 1H), 3.21-3.34 (br m, 2H), 3.94 (br s, 3H), 4.05-4.66 (br m, 3H), 6.53 (d, 1H, J=7.4 Hz), 7.06 (d, 1H, J=8.3 Hz), 7.08 (m, 1H), 7.19 (t, 1H, J=8.0 Hz); ¹³CNMR (methanol-d₄) δ 27.5, 32.7, 36.2, 38.3, 40.1, 51.7, 54.2, 55.1, 59.4, 99.0, 104.0, 104.6, 118.9, 125.6, 127.5, 138.0, 154.4, 162.6, 171.8, 175.2, 180.9; LRMS (ESI+) m/z: [M+H]⁺ Calcd for C₂₂H₂₅F₂N₅O₅ 477.2; Found 477.2.

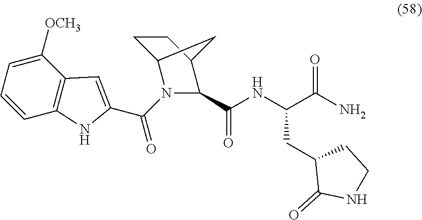 [SMILES: COc1cccc2[nH]c(C(=O)N3C4CCC(C4)[C@H]3C(=O)N[C@@H](C[C@@H]3CCNC3=O)C(N)=O)cc12]

(1R,3S,4S)—N—((S)-1-amino-1-oxo-3-((S)-2-oxopyrrolidin-3-yl)propan-2-yl)-2-(4-methoxy-1H-indole-2-carbonyl)-2-azabicyclo[2.2.1]heptane-3-carboxamide (Cmpd No. 58). Purified by silica gel chromatography using chloroform/methanol (0-4% methanol step gradient) to afford 8.4 mg (36% from resin 3) after overnight drying at 40° C. under house vacuum: ¹HNMR (methanol-d₄), rotamer ratio 61:39, line list given for the dominant rotamer, d 1.34-2.19 (several multiplets, 10H), 2.32-2.53 (m, 2H), 2.89-2.94 (m, 1H), 3.34-3.37 (m, 1H), 3.97 (s, 3H), 4.15 (s, 1H), 4.46 (dd, 1H, J=12.0 and 3.8 Hz), 5.00 (s, 1H), 6.55 (d, 1H, J=7.7 Hz), 7.07 (d, 1H, J=8.3 Hz), 7.11 (s, 1H), 7.19 (t, 1H, J=8.0 Hz); ¹³CNMR (methanol-d₄), line list given for the dominant rotamer, d 27.4 (2 carbons?), 30.6, 32.6, 35.8, 38.6, 40.2, 41.0, 51.9, 54.2, 59.6, 67.9, 98.8, 102.7, 104.7, 118.8, 125.1, 128.3, 137.7, 154.3, 162.1, 171.8, 175.5, 180.7.

### Conversion of Primary Amides to Nitriles.

Turning to FIG. 2 illustrates the synthetic scheme converting an amide to a nitrile. General procedure for the conversion of primary amides 1 to nitriles 6 (Method A, adapted from Tetrahedron Lett. 1988 29, 2155). To a 1-dr vial charged with 20-60 mmol of amide 1 with stir bar and fitted with a rubber septum stopper under dry argon gas is added via syringe 0.5-1.0 mL of dry dichloromethane (DCM, distilled from CaH₂ or dried over 3 Angstrom molecular sieves) followed by a solution of 1.4-1.9 equivalents of Burgess reagent in 0.5-0.75 mL of dry DCM also via syringe. The solution is stirred at room temperature for 2-3 hours and is then treated again with a solution of 1.4-1.9 equivalents of Burgess reagent in 0.5-0.75 mL of dry DCM. After stirring for 18-72 hours the solution is diluted with 15-20 mL of ethyl acetate or chloroform, is washed with 10 mL of 1.0 N HCl, once with 10 mL of saturated sodium carbonate, and once with 2 mL of brine. The organic phase is either dried over Na₂SO₄ (ethyl acetate) or passed through phase-separating paper (chloroform) and concentrated in vacuo to give crude nitriles which are purified by chromatography as described in the following characterizations.

General procedure for the conversion of primary amides 1 to nitriles 6 (Method B, adapted from Science 2021 374, 1586). To a 1-dr vial charged with 20-60 mmol of amide 1 with stir bar and fitted with a rubber septum stopper and under dry argon gas is added via syringe 0.50-1.0 mL of DCM followed by 0.5-1.0 mL of a solution of 2.8 equivalents of imidazole in pyridine. The contents are cooled to −35° C. using an acetonitrile/dry ice bath and then 6.8 equivalents of phosphorus oxychloride are added via syringe. The mixture is stirred at −35° C. to −10° C. for two hours and is then treated with 1 mL of 1N HCl. After 1-2 hours stirring at 0° C. the layers are separated, and the aqueous phase is extracted twice with DCM and the combined organics are dried over Na₂SO₄. Concentration gives the crude nitrile which is purified by chromatography as described in the following characterizations.

### Characterization of Nitriles

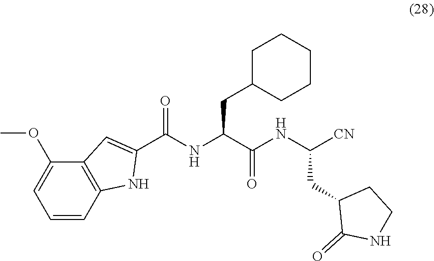 [SMILES: COc1cccc2[nH]c(C(=O)N[C@@H](CC3CCCCC3)C(=O)N[C@H](C#N)C[C@@H]3CCNC3=O)cc12]

N—((S)-1-(((S)-1-cyano-2-((S)-2-oxopyrrolidin-3-yl)ethyl)amino)-3-cyclohexyl-1-oxopropan-2-yl)-4-methoxy-1H-indole-2-carboxamide (Cmpd No. 28). Prepared by Method A. The crude sample was loaded onto a 500-mg HyperSep SI silica gel cartridge as a solution in 100 μL of chloroform. The column was eluted using chloroform and chloroform/methanol mobile phases (0-2.5% methanol) to give 8.8 mg (28%) of 28; ¹HNMR (methanol-d₄) δ 0.94-1.27 (m, 2H), 1.20-1.40 (m, 3H), 1.40-1.57 (m, 1H), 1.63-1.98 (m, 9H), 2.25-2.40 (m, 2H), 2.56-2.68 (m, 1H), 3.22-3.33 (m, 2H), 3.95 (s, 3H), 4.61 (dd, 1H, J=9.2 Hz and J=6.1 Hz), 5.07 (dd, 1H, J=5.9 Hz and J=10.2 Hz), 6.53 (d, 1H, J=7.7 Hz), 7.04 (d, 1H, J=8.3 Hz), 7.16 (t, 1H, J=8.0 Hz), 7.30 (s, 1H); ¹³CNMR (methanol-d₄) δ 25.8, 26.0, 26.2, 27.1, 32.2, 33.4, 33.7, 34.2, 37.7, 38.4, 38.8, 40.0, 51.4, 54.3, 98.9, 101.7, 104.8, 118.4, 118.7, 124.9, 128.8, 138.4, 154.3, 162.6, 173.7, 179.6.

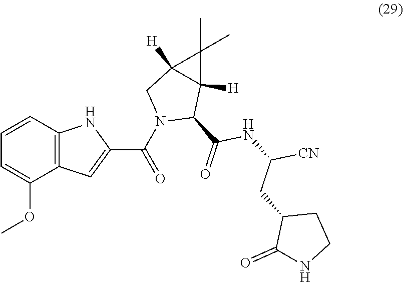 [SMILES: COc1cccc2[nH]c(C(=O)N3C[C@H]4[C@@H]([C@H]3C(=O)N[C@H](C#N)C[C@@H]3CCNC3=O)C4(C)C)cc12]

(1R,2S,5S)—N—((S)-1-cyano-2-((S)-2-oxopyrrolidin-3-yl)ethyl)-3-(4-methoxy-1H-indole-2-carbonyl)-6,6-dimethyl-3-azabicyclo[3.1.0]hexane-2-carboxamide (Cmpd No. 29). Prepared by Method A. Purification by silica gel chromatography using chloroform/methanol mobile phases (0-2% methanol) gave 9.8 mg (50%) of 29 after overnight drying under house vacuum at 40° C.; ¹HNMR (methanol-d₄), rotamer ratio 57:43, line list given for the dominant rotamer, d 0.98 (s, 3H), 1.12 (s, 3H), 1.49 (d, 1H, J=7.6 Hz), 1.74-1.77 (m, 1H), 1.79-1.87 (m, 1H), 1.94 (ddd, 1H, J=13.9, 9.6, and 5.7 Hz), 2.22-2.39 (m, 2H), 2.69 (ddd, 1H, J=14.9, 12.7, and 5.2 Hz), 3.29-3.32 (m, 2H), 3.96 (s, 3H), 4.00 (d, 1H, J=10.6 Hz), 4.31 (dd, 1H, J=10.5 and 5.6 Hz), 4.59 (s, 1H), 5.08 (m, 1H), 6.54 (d, 1H, J=7.7 Hz), 7.05 (d, 1H, J=8.3 Hz), 7.14 (s, 1H), 7.18 (t, 1H, J=8.1 Hz); ¹³CNMR (methanol-d₄), line list given for the dominant rotamer, d 11.4, 19.1, 24.9, 25.0, 28.3, 30.1, 33.8, 37.6, 38.5, 40.1, 48.7, 54.3, 61.8, 98.8, 103.9, 104.6, 118.4, 118.9, 125.4, 128.2, 137.8, 154.4, 161.3, 172.6, 179.7.

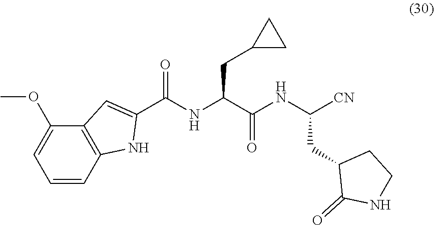 [SMILES: COc1cccc2[nH]c(C(=O)N[C@@H](CC3CC3)C(=O)N[C@H](C#N)C[C@@H]3CCNC3=O)cc12]

N—((S)-1-(((S)-1-cyano-2-((S)-2-oxopyrrolidin-3-yl)ethyl)amino)-3-cyclopropyl-1-oxopropan-2-yl)-4-methoxy-1H-indole-2-carboxamide (Cmpd No. 30). Prepared by Method A. The crude sample was thoroughly mixed with 200 mg of silica gel and methanol was dried and was applied to the top of a 500-mg HyperSep Si cartridge of silica gel. The column was then eluted with chloroform/methanol mobile phases (0-10% methanol) to afford 5.6 mg (24%) of 30 after overnight drying under house vacuum at 40° C.; ¹HNMR (methanol-d₄), d −0.05-0.06 (m, 2H), 0.35 (dd, 2H, J=18.2 Hz and 10.1 Hz), 0.61-0.74 (m, 1H), 1.43-1.53 (m, 1H), 1.57-1.78 (2m, 3H), 2.07-2.22 (m, 2H), 2.41-2.51 (m, 1H), 3.04-3.12 (m, 2H), 3.74 (s, 3H), 4.36 (t, 1H, J=7.35 Hz), 4.88 (dd, 1H, J=10.2 Hz, J=5.9 Hz), 6.32 (d, 1H, J=7.7 Hz), 6.83 (d, 1H, J=8.3 Hz), 6.96 (t, 1H, J=8.0 Hz), 7.08 (s, 1H); ¹³CNMR (methanol-d₄), d 1.4, 1.5, 5.1, 24.9, 31.5, 34.1, 35.5, 36.1, 37.8, 52.1, 52.1, 96.7, 99.4, 102.5, 116.1, 116.5, 122.7, 126.6, 136.2, 152.0, 160.3, 171.0, 177.4.

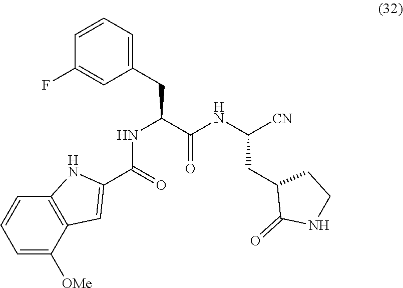

N—((S)-1-(((S)-1-cyano-2-((S)-2-oxopyrrolidin-3-yl)ethyl)amino)-3-(3-fluorophenyl)-1-oxopropan-2-yl)-4-methoxy-1H-indole-2-carboxamide (Cmpd No. 32). Prepared by Method B. The crude sample was loaded onto a dry 500-mg cartridge of HyperSep SI silica gel as solution in 0.200 mL of methanol and then the column was dried overnight using a stream of nitrogen gas. The column was eluted using chloroform and chloroform/methanol mobile phases (1-2% methanol) to give 4.4 mg (29%) of 32. ¹H NMR (methanol-d₄) δ 1.62-1.81 (m, 2H), 2.11-2.24 (m, 2H), 2.39-2.50 (m, 1H), 3.03 (dd, 1H, J=8.0 Hz and J=13.3 Hz), 3.10-3.19 (m, 3H), 3.83 (s, 3H), 4.62 (t, 1H, J=7.4 Hz), 4.90 (dd, 1H, J=5.8 Hz and J=9.8 Hz), 6.40 (d, 1H, J=7.7 Hz), 6.84 (dt, 1H, J=2.6 Hz and J=8.9 Hz), 6.91 (d, 1H, J 10=8.2 Hz), 6.94-7.07 (m, 3H), 7.11 (s, 1H), 7.19 (q, 1H, J=7.4 Hz)); ¹³C NMR (methanol-d₄) δ 27.1, 33.6, 36.8, 37.7, 38.4, 40.0, 54.3, 54.9, 98.9, 101.6, 104.7, 113.3 (d, ²J_{CF}=21.3 Hz), 115.7 (d, ²J_{CF}=21.5 Hz), 118.1, 118.7, 124.9 (d, ⁴J_{CF}=2.8 Hz), 125.0, 128.7, 129.9 (d, ³J_{CF}=8.3 Hz), 138.4, 139.6 (d, ³J_{CF}=7.6 Hz), 154.2, 162.4, 162.9 (d, ¹J_{CF}=245 Hz), 172.0, 179.6.

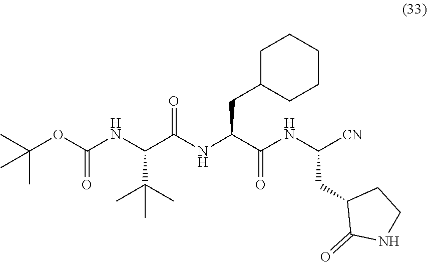 [SMILES: CC(C)(C)OC(=O)N[C@H](C(=O)N[C@@H](CC1CCCCC1)C(=O)N[C@H](C#N)C[C@@H]1CCNC1=O)C(C)(C)C]

tert-butyl ((S)-1-(((S)-1-(((S)-1-cyano-2-((S)-2-oxopyrrolidin-3-yl)ethyl)amino)-3-cyclohexyl-1-oxopropan-2-yl)amino)-3,3-dimethyl-1-oxobutan-2-yl)carbamate (Cmpd No. 33): Prepared by Method A. The crude sample was loaded onto a 500-mg HyperSep SI silica gel cartridge as a solution in 2-3 mL of chloroform. The column was eluted using chloroform and chloroform/methanol mobile phases (0-2% methanol) to give 8.1 mg (64%) of 33. ¹HNMR (methanol-d₄) δ 0.99 (s, 9H), 1.00-1.37 (m, 6H), 1.47 (s, 9H), 1.55-1.94 (m, 9H), 2.25-2.40 (m, 2H), 2.54-2.66 (m, 1H), 3.23-3.36 (m, 2H), 3.93 (s, 1H), 4.36 (t, 1H, J=7.7 Hz), 5.03 (dd, 1H, J=5.5 Hz and J=10.6 Hz); ¹³CNMR (methanol-d₄) δ 25.7, 25.9, 26.0, 26.1, 27.1, 27.3, 32.3, 33.2, 33.9, 33.9, 34.0, 37.5, 38.1, 38.7, 40.0, 51.1, 62.1, 79.2, 118.4, 156.4, 172.0, 173.0, 179.5.

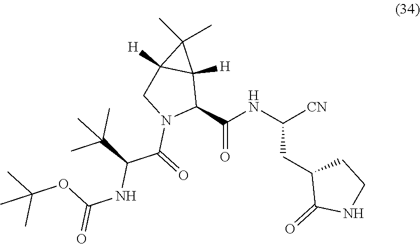 [SMILES: CC(C)(C)OC(=O)N[C@H](C(=O)N1C[C@H]2[C@@H]([C@H]1C(=O)N[C@H](C#N)C[C@@H]1CCNC1=O)C2(C)C)C(C)(C)C]

tert-Butyl ((S)-1-((1R,2S,5S)-2-(((S)-1-cyano-2-((S)-2-oxopyrrolidin-3-yl)ethyl)carbamoyl)-6,6-dimethyl-3-azabicyclo[3.1.0]hexan-3-yl)-3,3-dimethyl-1-oxobutan-2-yl)carbamate (Cmpd No. 34). Prepared by Method A. Purification by silica gel chromatography using chloroform/methanol mobile phases (0-1% methanol) gave 19.5 mg (50%) of 34 after overnight drying under house vacuum at 40° C.; ¹HNMR (CDCl₃), rotamer ratio 85:15, line list given for the dominant rotamer, d 0.89 (s, 3H), 0.98 (s, 9H), 1.06 (s, 3H), 1.39 (s, 9H), 1.55 (s, 2H), 1.78-1.87 (m, 1H), 1.89-1.97 (m, 1H), 2.30-2.45 (m, 2H), 2.53-2.62 (m, 1H), 3.31-3.35 (m, 2H), 3.90 (br d, 1H, J=10.0 Hz), 3.99 (d, 1H, J=10.3 Hz), 4.20 (d, 1H, J=10.2 Hz), 4.30 (s, 1H), 4.96 (ddd, 1H, J=13.3, 10.2, and 7.0 Hz), 6.31 (s, 1H, NH), 8.12 (d, 1H, J=7.4 Hz, NH); ¹³CNMR (CDCl₃), line list given for the dominant rotamer, d 12.5, 19.2, 26.2, 26.4, 27.9, 28.2, 28.5, 30.0, 34.1, 34.9, 37.6, 39.1, 40.4, 48.3, 58.7, 60.4, 79.7, 118.4, 155.9, 171.3, 171.6, 179.0.

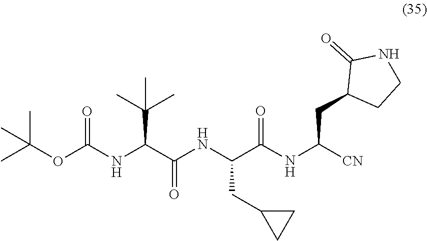 [SMILES: CC(C)(C)OC(=O)N[C@H](C(=O)N[C@@H](CC1CC1)C(=O)N[C@H](C#N)C[C@@H]1CCNC1=O)C(C)(C)C]

tert-Butyl ((S)-1-(((S)-1-(((S)-1-cyano-2-((S)-2-oxopyrrolidin-3-yl)ethyl)amino)-3-cyclopropyl-1-oxopropan-2-yl)amino)-3,3-dimethyl-1-oxobutan-2-yl)carbamate (Cmpd No. 35). Prepared by Method A. Purification by silica gel chromatography using chloroform/methanol mobile phases (0-2% methanol) gave 13.2 mg (58%) of 35 after overnight drying under house vacuum at 40° C.; ¹HNMR (CDCl₃), d 0.08-0.14 (m, 2H), 0.42-0.54 (m, 2H), 0.62-0.76 (m, 1H), 0.98 (s, 9H), 1.42 (s, 9H), 1.55-1.76 (m, 2H), 1.76-1.90 (m, 1H), 1.91-2.07 (m, 1H), 2.28-2.55 (m, 3H), 3.25-3.43 (m, 2H), 3.90 (d, 1H, J=9.41 Hz), 4.61 (dd, 1H, J=14.79 Hz, J=6.91 Hz), 4.84-5.01 (m, 1H), 5.29 (d, 1H, J=9.38 Hz), 6.84 (s, 1H), 7.09 (d, 1H, J=7.55 Hz), 8.23 (d, 1H, J=6.64 Hz); ¹³CNMR (CDCl₃), d 4.3, 4.5, 7.2, 26.6, 27.9, 28.3, 34.1, 34.5, 37.4, 37.8, 38.7, 40.5, 53.6, 62.4, 80.0, 118.1, 155.9, 170.9, 171.9, 179.0.

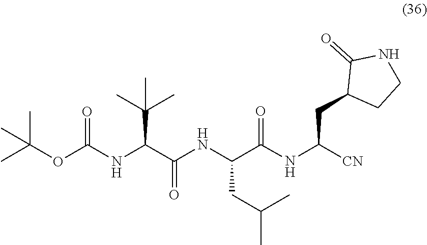 [SMILES: CC(C)C[C@H](NC(=O)[C@@H](NC(=O)OC(C)(C)C)C(C)(C)C)C(=O)N[C@H](C#N)C[C@@H]1CCNC1=O]

tert-Butyl ((S)-1-(((S)-1-(((S)-1-cyano-2-((S)-2-oxopyrrolidin-3-yl)ethyl)amino)-4-methyl-1-oxopentan-2-yl)amino)-3,3-dimethyl-1-oxobutan-2-yl)carbamate (Cmpd No. 36). Prepared by Method A. Purification by silica gel chromatography using chloroform/methanol mobile phases (0-2% methanol) gave 12.6 mg (61%) of 36 after overnight drying under house vacuum at 40° C.; ¹HNMR (CDCl₃), 0.92 (d, 3H, J=4.8 Hz), 0.94 (d, 3H, J=5.09 Hz), 0.99 (s, 9H), 1.43 (s, 9H), 1.48-1.57 (m, 1H), 1.57-1.71 (m, 2H), 1.72-1.89 (m, 1H), 1.9-2.0 (m, 1H), 2.3-2.5 (m, 3H), 3.22-3.46 (m, 2H), 3.84 (d, 1H, J=8.42 Hz), 4.51-4.65 (m, 1H), 4.89 (s, 1H), 5.22 (d, 1H, J=9.94 Hz), 6.86 (s, 1H), 6.96 (d, 1H, J=6.86 Hz), 8.13 (d, 1H, J=6.27 Hz); ¹³CNMR (CDCl₃), d 21.8, 22.9, 24.8, 26.5, 27.9, 28.3, 34.2, 34.3, 37.8, 38.7, 40.5, 41.7, 51.6, 62.6, 80.2, 118.2, 156.1, 171.1, 172.4, 179.0.

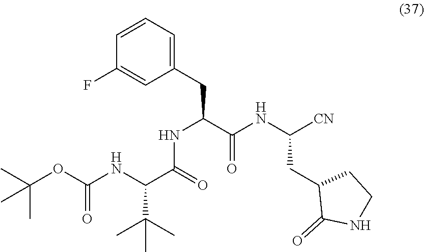

tert-Butyl ((S)-1-(((S)-1-(((S)-1-cyano-2-((S)-2-oxopyrrolidin-3-yl)ethyl)amino)-3-(3-fluorophenyl)-1-oxopropan-2-yl)amino)-3,3-dimethyl-1-oxobutan-2-yl)carbamate (Cmpd No. 37). Prepared by Method A. The crude sample was loaded onto a 500-mg cartridge of HyperSep SI silica gel as solution in 0.175 mL of chloroform (equilibrated column in chloroform). The column was eluted using chloroform and chloroform/methanol mobile phases (1-2% methanol) to give 21.1 mg (66%) of 37. ¹H NMR (methanol-d₄) δ 0.82 (s, 9H), 1.34 (s, 9H), 1.64-1.75 (m, 2H), 2.08-2.22 (m, 2H), 2.34-2.45 (m, 1H), 2.91 (dd, 1H, J=7.6 Hz and J=13.6 Hz), 2.97 (dd, 1H, J=8.1 and J=13.6 Hz), 3.11-3.19 (m, 2H), 3.79 (s, 1H), 4.39 (t, 1H, J=7.7 Hz), 4.82 (dd, 1H, J=5.6 Hz and J=5.6 Hz), 6.85 (dt, 1H, J=2.1 Hz and J=8.4 Hz), 6.92 (d, 1H, J=9.8 Hz), 6.96 (d, 1H, J=7.7 Hz), 7.15-7.23 (m, 1H); ¹³C NMR (methanol-d₄) δ 25.7, 27.1, 27.3, 33.8, 33.9, 36.8, 37.5, 38.1, 40.0, 54.7, 62.2, 79.2, 113.3 (d, ²J_{CF}=21.1 Hz), 115.7 (d, ²J_{CF}=21.7 Hz), 117.9 (CN), 124.8 (d, ⁴J_{CF}=2.7 Hz), 129.9 (d, ³J_{CF}=8.5 Hz), 139.1 (d, ³J_{CF}=7.5 Hz), 156.3, 162.9 (d, ¹J_{CF}=245.0 Hz), 171.4, 171.8, 179.4.

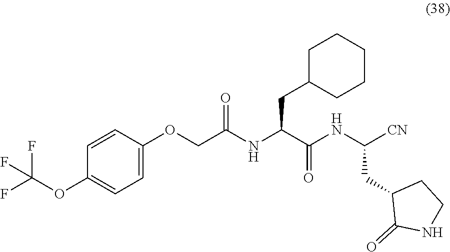 [SMILES: N#C[C@H](C[C@@H]1CCNC1=O)NC(=O)[C@H](CC1CCCCC1)NC(=O)COc1ccc(OC(F)(F)F)cc1]

(S)—N—((S)-1-cyano-2-((S)-2-oxopyrrolidin-3-yl)ethyl)-3-cyclohexyl-2-(2-(4-(trifluoromethoxy)phenoxy)-acetamido)propenamide (Cmpd No. 38): Prepared by Method A. The crude sample was loaded onto a 500 mg HyperSep SI silica gel cartridge as a solution in 2-3 mL of chloroform. The column was eluted using chloroform and chloroform/methanol mobile phases (0-2% methanol) to give 11.9 mg (41%) of 38. ¹HNMR (CDCl₃) δ 0.78-1.03 (m, 2H), 1.03-1.32 (m, 5H), 1.43-2.02 (m, 10H), 2.29-2.50 (m, 3H), 3.27-3.44 (m, 2H), 4.52 (s, 2H), 4.72 (br s, 1H), 4.78 (br s, 1H), 6.31 (s, 1H), 6.94 (d, 2H, J=9.1 Hz), 7.03 (d, 1H, J=8.5 Hz), 7.19 (d, 2H, J=8.9 Hz), 8.59 (d, 1H, J=6.3 Hz); ¹³CNMR (CDCl₃) d 26.0, 26.1, 26.3, 28.3, 32.6, 33.5, 33.5, 34.0, 38.1, 39.5, 40.2, 40.6, 50.4, 67.6, 115.7, 118.2, 120.5 (q, ¹J_{CF}=257 Hz), 122.7, 143.8, 155.6, 167.9, 172.3, 179.0.

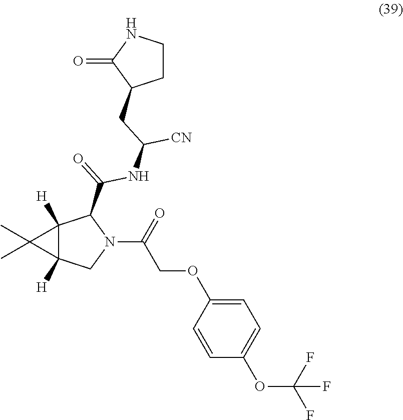 [SMILES: CC1(C)[C@@H]2[C@@H](C(=O)N[C@H](C#N)C[C@@H]3CCNC3=O)N(C(=O)COc3ccc(OC(F)(F)F)cc3)C[C@@H]21]

(1R,2S,5S)—N—((S)-1-cyano-2-((S)-2-oxopyrrolidin-3-yl)ethyl)-6,6-dimethyl-3-(2-(4-(trifluoromethoxy)-phenoxy)acetyl)-3-azabicyclo[3.1.0]hexane-2-carboxamide (Cmpd No. 39). Prepared by Method A. Purification by silica gel chromatography using chloroform/methanol mobile phases (0-2% methanol) gave 5.6 mg (36%) of 39 after overnight drying under house vacuum at 40° C.; ¹HNMR (methanol-d₄), rotamer ratio 85:15, line list given for the dominant rotamer, d 1.01 (s, 3H), 1.13 (s, 3H), 1.46 (d, 1H, J=7.6 Hz), 1.70 (dd, 1H, J=7.5 and 5.5 Hz), 1.71-1.79 (m, 1H), 1.89 (ddd, 1H, J=14.0, 10.0, and 5.3 Hz), 2.15-2.21 (m, 1H), 2.32 (ddd, 1H, J=13.9, 11.0, and 4.9 Hz), 2.57-2.63 (m, 1H), 3.07 (ddd, 1H, J=16.7, 9.5, and 7.2 Hz), 3.19 (dt, 1H, J=9.7 and 2.0 Hz), 3.66 (d, 1H, J=10.4 Hz), 3.97 (dd, 1H, J=10.4 and 5.5 Hz), 4.31 (s, 1H), 4.75 (d, 1H, J=15.5 Hz), 4.81 (d, 1H, J=15.5 Hz), 5.05 (dd, 1H, J=10.9 and 5.2 Hz), 6.99 (d, 2H, J=9.2 Hz), 7.20 (d, 2H, J=8.5 Hz; ¹³CNMR (methanol-d₄), line list given for the dominant rotamer, d 11.5, 19.2, 24.9, 27.1, 27.8, 30.5, 33.9, 37.5, 38.2, 39.9, 45.9, 60.7, 66.1, 115.5, 118.3, 120.6 (q, ¹J_{CF}=254 Hz), 122.0, 143.0, 156.8, 167.4, 172.0, 179.6.

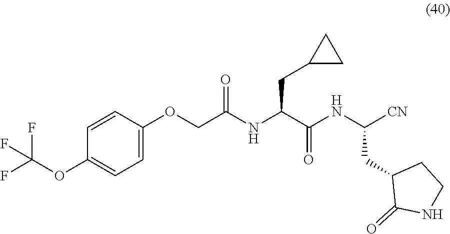 [SMILES: N#C[C@H](C[C@@H]1CCNC1=O)NC(=O)[C@H](CC1CC1)NC(=O)COc1ccc(OC(F)(F)F)cc1]

(S)—N—((S)-1-cyano-2-((S)-2-oxopyrrolidin-3-yl)ethyl)-3-cyclopropyl-2-(2-(4-(trifluoromethoxy)phenoxy)-acetamido)propenamide (Cmpd No. 40): Prepared by Method A. The crude sample was loaded onto a 500 mg Hypersep SI silica gel as a solution in 2-3 mL of chloroform. The column was eluted using chloroform and chloroform/methanol phases (0-2% methanol) to give 4.5 mg (22% over two steps) of 40. ¹HNMR (methanol-d₄) d −0.06-0.07 (m, 2H), 0.31-0.41 (m, 2H), 0.53-0.65 (m, 1H), 1.40-1.50 (m, 1H), 1.60-1.82 (m, 3H), 2.07-2.21 (m, 2H), 2.39-2.50 (m, 1H), 3.03-3.17 (m, 2H), 4.28 (t, 1H, J=7.2 Hz), 4.45 (d, 1H, J=14.9 Hz), 4.50 (d, 1H, J=14.9 Hz), 4.91 (dd, 1H, J=5.9 Hz and J=10.3 Hz), 6.93 (d, 2H, J=9.1 Hz), 7.09 (d, 2H, J=8.89 Hz); ¹³CNMR (methanol-d₄) δ 1.42, 1.51, 4.86, 25.1, 31.6, 34.2, 35.6, 36.3, 37.9, 51.7, 64.8, 113.5, 116.2, 118.5 (q, ¹J_{CF}=255 Hz), 120.1, 141.2, 154.5, 167.2, 170.4, 177.5.

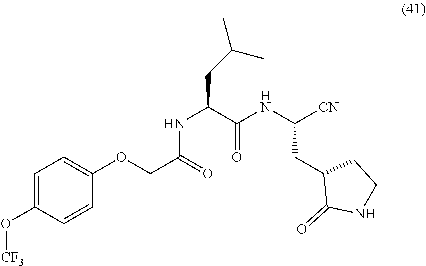 [SMILES: CC(C)C[C@H](NC(=O)COc1ccc(OC(F)(F)F)cc1)C(=O)N[C@H](C#N)C[C@@H]1CCNC1=O]

(S)—N—((S)-1-cyano-2-((S)-2-oxopyrrolidin-3-yl)ethyl)-4-methyl-2-(2-(4-(trifluoromethoxy)phenoxy)-acetamido)pentanamide (Cmpd No. 41). Prepared by Method A. Compound 41 was prepared on a 38.6 mmol scale. It was purified by silica gel chromatography using chloroform/methanol (0-1% methanol step gradient) to afford 13.2 mg (70%) of 41 after overnight drying at 40° C. under house vacuum: ¹HNMR (CDCl₃), d 0.94 (d, 6H, J=5.5 Hz), 1.55-1.64 (m, 2H), 1.67-1.74 (m, 1H), 1.80-1.89 (m, 1H), 1.96-2.02 (m, 1H), 2.34-2.47 (m, 3H), 3.31-3.41 (m, 2H), 4.49 (s, 2H), 4.69-4.78 (m, 2H), 6.51 (br s, 1H), 6.94 (d, 2H, J=9.2 Hz), 7.08 (d, 1H, J=8.7 Hz), 7.18 (d, 1H, J=8.8 Hz), 8.65 (d, 1H, J=6.3 Hz); ¹³CNMR (CDCl₃) d 22.0, 22.8, 24.7, 28.2, 33.5, 38.0, 39.4, 40.6, 41.8, 50.9, 67.6, 115.7, 118.2, 120.5 (q, ¹J_{CF}=256 Hz), 122.7, 143.8 (q, ³J_{CF}=2.1 Hz), 155.5, 167.9, 172.2, 179.0.

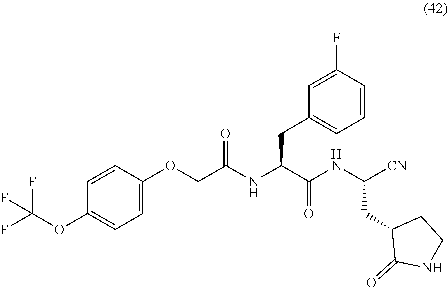 [SMILES: N#C[C@H](C[C@@H]1CCNC1=O)NC(=O)[C@H](Cc1cccc(F)c1)NC(=O)COc1ccc(OC(F)(F)F)cc1]

(S)—N—((S)-1-cyano-2-((S)-2-oxopyrrolidin-3-yl)ethyl)-3-(3-fluorophenyl)-2-(2-(4-(trifluoromethoxy)-phenoxy)acetamido)propenamide (Cmpd No. 42). Prepared by Method A. Purification by silica gel chromatography using chloroform/methanol mobile phases (0-3% methanol) gave 7.2 mg (53%) of 42 after overnight drying under house vacuum at 40° C.; ¹HNMR (methanol-d₄) d 1.57-1.88 (m, 2H), 2.05-2.24 (m, 2H), 2.27-2.48 (m, 1H), 2.89-2.99 (m, 1H), 3.04-3.19 (m, 3H), 4.40 (d, 1H, J=15.02 Hz), 4.46 (d, 1H, J=15.7 Hz), 4.53 (dd, 1H, J=8.2 Hz, J=6.3 Hz), 4.88 (dd, 1H, J=10.3 Hz, J=6.2 Hz), 6.81-6.96 (m, 4H), 7.08 (d, 2H, J=8.7 Hz), 7.17 (dd, 1H, J=13.9 Hz, J=7.9 Hz); ¹³CNMR (methanol-d₄), d 27.2, 33.6, 36.7, 37.7, 38.5, 40.0, 54.2, 66.9, 113.4 (d, ²J_{CF}=21.3 Hz), 115.6, 115.7 (d, ²J_{CF}=21.9 Hz), 118.1, 120.6 (q, ¹J_{CF}=255 Hz), 122.2, 124.9 (d, ⁴J_{CF}=2.7 Hz), 129.9 (d, ³J_{CF}=8.3 Hz), 139.2 (d, ³J_{CF}=7.6 Hz), 143.3, 156.5, 162.9 (d, ¹J_{CF}=244.8 Hz), 169.4, 171.4, 179.6.

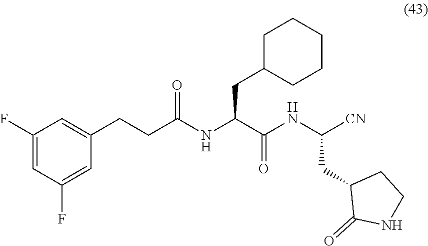 [SMILES: N#C[C@H](C[C@@H]1CCNC1=O)NC(=O)[C@H](CC1CCCCC1)NC(=O)CCc1cc(F)cc(F)c1]

(S)—N—((S)-1-cyano-2-((S)-2-oxopyrrolidin-3-yl)ethyl)-3-cyclohexyl-2-(3-(3,5-difluorophenyl)propan-amido)propenamide (Cmpd No. 43): Prepared by Method A. The crude sample was loaded onto a 500 mg Hypersep SI silica gel as a solution in 2-3 mL of chloroform. The column was eluted using chloroform and chloroform/methanol phases (0-2% methanol) to give 6.3 mg (41% over two steps) of 43. ¹HNMR (methanol-d₄) δ 0.71-0.93 (m, 2H), 0.98-1.25 (m, 5H), 1.36-1.49 (m, 2H), 1.50-1.65 (m, 4H), 1.65-1.85 (m, 2H), 2.13-2.25 (m, 2H), 2.46 (t and m, 3H, J=7.4 Hz), 2.82 (t, 2H, J=7.4 Hz), 3.15-3.26 (m, 2H), 4.19 (t, 1H, J=7.6 Hz), 4.90 (dd, 1H, J=6.0 Hz and J=10.1 Hz), 6.65 (t, 1H, J=9.2 Hz), 6.73 (d, 2H, J=6.6 Hz); ¹³CNMR (methanol-d₄) δ 25.7, 25.9, 26.1, 27.2, 30.7, 32.0, 33.4, 33.6, 33.9, 36.0, 37.7, 38.4, 38.9, 40.1, 51.1, 101.0 (t, ²J_{CF}=25.8 Hz), 110.9 (dd, ²J_{CF}=18.2 Hz and ⁴J_{CF}=6.6 Hz), 118.4, 145.3 (t, ³J_{CF}=9.1 Hz), 163.1 (dd, ¹J_{CF}=247 Hz and ³J_{CF}=13.1 Hz), 173.2, 173.5, 179.6.

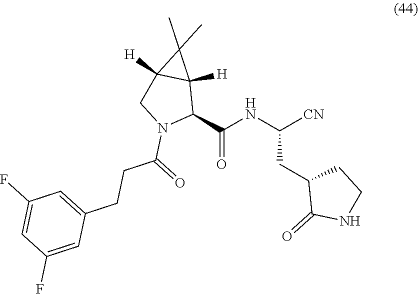 [SMILES: CC1(C)[C@@H]2[C@@H](C(=O)N[C@H](C#N)C[C@@H]3CCNC3=O)N(C(=O)CCc3cc(F)cc(F)c3)C[C@@H]21]

(1R,2S,5S)—N—((S)-1-cyano-2-((S)-2-oxopyrrolidin-3-yl)ethyl)-3-(3-(3,5-difluorophenyl)propanoyl)-6,6-dimethyl-3-azabicyclo[3.1.0]hexane-2-carboxamide (Cmpd No. 44). Prepared by Method A. A vessel containing 18 (18.4 mg, 38.6 mol) was purged with argon gas prior to capping with a rubber septum and solubilizing the starting material with 1 mL of dichloromethane (distilled over calcium hydride). The solution was stirred while adding Burgess reagent (29.4 mg, 123.4 mol, 3.2 eq) to the vessel in two aliquots over a two-hour time period. The reaction mixture was allowed to stir for three days, with the resulting reaction mixture transferred to a separatory funnel. The solution was washed with 10 mL of 1N hydrochloric acid followed by 10 mL of saturated sodium carbonate and 3 mL of brine. The organic layer was allowed to dry over sodium sulfate. Concentration gave crude nitrile which was solubilized in 250 μL of 97/3 chloroform/methanol and dispensed onto a 500-mg HyperSep SI cartridge of silica gel. The material was then eluted with chloroform/methanol mobile phases (0-3% methanol) in step gradient manner, affording 7.6 mg (43%) of 44 after overnight drying at 40° C. under house vacuum: ¹HNMR (methanol-d₄), rotamer ratio of 81:19, line list given for the dominant rotamer, d 0.87 (s, 3H), 1.06 (s, 3H), 1.37 (d, 1H, J=7.6 Hz), 1.54-1.59 (m, 1H), 1.74-1.99 (m, 2H), 2.17-2.41 (m, 2H), 2.47-2.70 (m, 3H), 2.89 (t, 2H, J=7.3 Hz), 3.16-3.29 (m, 2H), 3.51 (d, 1H, J=10.5 Hz), 3.87 (dd, 1H, J=5.5 and 5.0 Hz, 4.20 (s, 1H), 4.97-5.07 (m, 1H), 6.68-6.76 (m, 1H), 6.77-6.87 (m, 2H); ¹³CNMR (methanol-d₄), line list given for the dominant rotamer, d 11.4, 19.0, 24.9, 27.1, 27.5, 29.8, 31.0, 33.8, 34.7, 37.7, 38.4, 40.1, 60.4, 100.8 (t, ²J_{CF}=25.7 Hz), 111.0 (dd, ²J_{CF}=18.4 Hz and ⁴J_{CF}=6.5 Hz), 118.3, 145.5 (t, ³J_{CF}=8.9 Hz), 163.1 (dd, ¹J_{CF}=246 Hz and ³J_{CF}=13.1 Hz), 171.3, 172.5, 179.7.

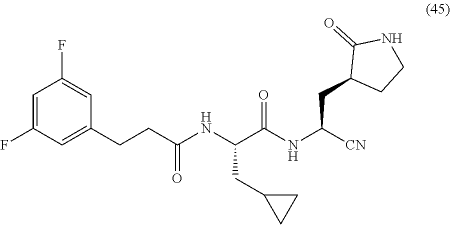 [SMILES: N#C[C@H](C[C@@H]1CCNC1=O)NC(=O)[C@H](CC1CC1)NC(=O)CCc1cc(F)cc(F)c1]

(S)—N—((S)-1-cyano-2-((S)-2-oxopyrrolidin-3-yl)ethyl)-3-cyclopropyl-2-(3-(3,5-difluorophenyl)-propanamido)propenamide (Cmpd No. 45). Prepared by Method A. Purification by silica gel chromatography using chloroform/methanol mobile phases (0-3% methanol) gave 3.4 mg (17%) of 45 after overnight drying under house vacuum at 40° C.; ¹HNMR (methanol-d₄) d −0.08-0.04 (m, 2H), 0.27-0.40 (m, 2H), 0.46-0.60 (m, 1H), 1.24-1.36 (m, 1H), 1.51-1.63 (m, 1H), 1.63-1.84 (m, 2H), 2.09-2.22 (m, 2H), 2.35-2.51 (m, 3H), 2.72-2.85 (m, 2H), 3.13-3.17 (m, 2H), 4.14 (t, 1H, J=7.09 Hz), 4.89 (dd, 1H, J=10.3 and 6.0 Hz), 6.61 (tt, 1H, J=11.4 and 2.2 Hz), 6.69 (d, 2H, J=6.6 Hz); ¹³CNMR (methanol-d₄), d 1.3, 1.4, 4.8, 24.9, 28.5, 31.5, 33.9, 34.2, 35.4, 36.1, 37.8, 51.8, 98.7 (t, ²J_{CF}=25.8 Hz), 108.7 (dd, ²J_{CF}=18.4 Hz, ⁴J_{CF}=6.6 Hz), 116.1, 143.1 (t, ³J_{CF}=9.0 Hz), 160.9 (dd, ¹J_{CF}=246 Hz and ³J_{CF}=12.9 Hz), 170.7, 170.9, 177.4.

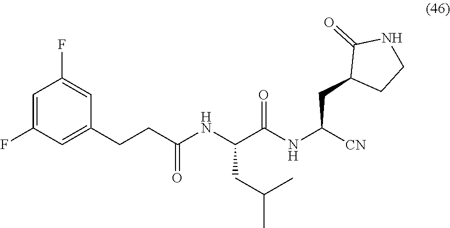 [SMILES: CC(C)C[C@H](NC(=O)CCc1cc(F)cc(F)c1)C(=O)N[C@H](C#N)C[C@@H]1CCNC1=O]

(S)—N—((S)-1-cyano-2-((S)-2-oxopyrrolidin-3-yl)ethyl)-2-(3-(3,5-difluorophenyl)propanamido)-4-methyl-pentanamide (46). Prepared by Method A. Purification by silica gel chromatography using chloroform/methanol mobile phases (0-3% methanol) gave 7.3 mg (31%) of 46 after overnight drying under house vacuum at 40° C.; ¹HNMR (CDCl₃), d 0.92 (2d, 6H, J=6.0 Hz), 1.41-1.56 (m, 2H), 1.57-1.68 (m, 1H), 1.81-1.93 (m, 1H), 1.94-2.05 (m, 1H), 2.33-2.49 (m, 3H), 2.49-2.64 (m, 2H), 2.87-3.02 (m, 2H), 3.31-3.49 (m, 2H) 4.59-4.68 (m, 1H), 4.69-4.80 (m, 1H), 6.36 (d, 1H, J=9.0 Hz), 6.55-6.69 (m, 2H), 6.73 (d, 2H, J=7.1 Hz), 8.63 (d, 1H, J=5.9 Hz); ¹³CNMR (CDCl₃), d 21.9, 22.8, 24.7, 28.3, 29.7, 31.0, 33.4, 37.3, 39.5, 40.6, 41.9, 51.2, 101.7 (t, ²J_{CF}=25.4 Hz), 111.2 (dd, ²J_{CF}=18.0 Hz, ⁴J_{CF}=6.5 Hz), 118.2, 144.5 (d, ³J_{CF}=9.1 Hz), 163.0 (dd, ¹J_{CF}=248, ³J_{CF}=12.7 Hz), 171.5, 172.9, 179.2.

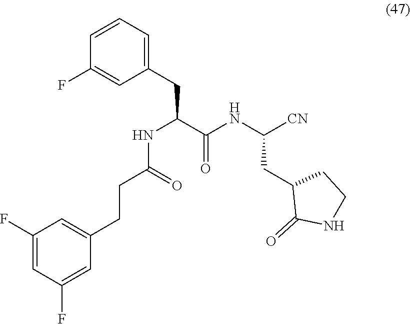 [SMILES: N#C[C@H](C[C@@H]1CCNC1=O)NC(=O)[C@H](Cc1cccc(F)c1)NC(=O)CCc1cc(F)cc(F)c1]

(S)—N—((S)-1-cyano-2-((S)-2-oxopyrrolidin-3-yl)ethyl)-2-(3-(3,5-difluorophenyl)propanamido)-3-(3-fluorophenyl)propenamide (Cmpd No. 47). Prepared by Method A. The crude sample was loaded onto a 500-mg cartridge of HyperSep SI silica gel as solution in 4 mL of chloroform that was heated to reflux (equilibrated column in chloroform). The column was eluted using chloroform and chloroform/methanol mobile phases (1-2% methanol) to give 13.5 mg of 47. This material was recrystallized from 9/1 chloroform/methanol to give 3.5 mg (15%) of 47. ¹H NMR (methanol-d₄) δ 1.63-1.80 (m, 2H), 2.06-2.23 (m, 2H), 2.32-2.47 (m, 3H), 2.74 (t, 2H, J=7.5 Hz), 2.81 (dd, 1H, J=J=13.7 and 8.4 Hz), 2.96 (dd, 1H, J=J=13.7 and 6.8 Hz), 3.13-3.21 (m, 2H), 4.39 (t, 1H, J=7.5 Hz), 4.85 (dd, 1H, J=10.0 and J=6.1 Hz), 6.58-6.70 (2m, 3H), 6.81-6.94 (2m, 3H), 7.14-7.22 (m, 1H); ¹³C NMR (methanol-d₄) δ 27.2, 30.7, 33.6, 36.1, 36.9, 37.7, 38.4, 40.0, 54.6, 100.9 (t, ²J_{CF}=25.8 Hz), 110.8 (dd, ²J_{CF}=18.4 Hz and ⁴J_{CF}=6.6 Hz), 113.3 (d, ²J_{CF}=21.2 Hz), 115.6 (d, ²J_{CF}=21.7 Hz), 118.0 (CN), 124.7 (d, ⁴J_{CF}=2.8 Hz), 129.8 (d, ³J_{CF}=8.4 Hz), 139.3 (d, ³J_{CF}=7.4 Hz), 145.2 (t, ³J_{CF}=9.1 Hz), 162.8 (d, ¹J_{CF}=244 Hz), 163.0 (dd, ¹J_{CF}=247 Hz and ³J_{CF}=12.9 Hz), 171.8, 173.1, 179.5.

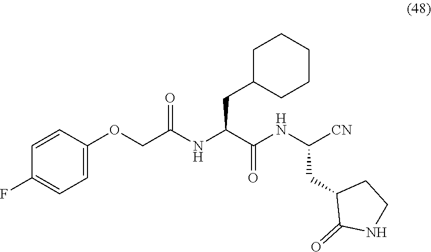 [SMILES: N#C[C@H](C[C@@H]1CCNC1=O)NC(=O)[C@H](CC1CCCCC1)NC(=O)COc1ccc(F)cc1]

(S)—N—((S)-1-cyano-2-((S)-2-oxopyrrolidin-3-yl)ethyl)-3-cyclohexyl-2-(2-(4-fluorophenoxy)acetamido)-propanamide (Cmpd No. 48). Prepared by Method A. Purification by silica gel chromatography using chloroform/methanol mobile phases (0-2% methanol) gave 6.8 mg (46%) of 48 after overnight drying under house vacuum at 40° C.; ¹HNMR (CDCl₃), d 0.84-1.02 (m, 2H), 1.06-1.31 (m, 5H), 1.51-1.82 (m, 6H), 1.82-1.93 (m, 1H), 1.94-2.05 (m, 1H), 2.31-2.50 (m, 3H), 3.30-3.42 (m, 2H), 4.47 (s, 2H), 4.63-4.72 (m, 1H), 4.72-4.81 (m, 1H), 6.29 (s, 1H), 6.85-6.92 (m, 2H), 6.97-7.05 (m, 3H), 8.55 (d, 1H, J 6.6 Hz); ¹³CNMR (CDCl₃), d 26.0, 26.1, 26.3, 28.3, 32.6, 33.5, 33.5, 34.0, 38.1, 39.5, 40.2, 40.6, 50.4, 67.9, 115.9 (d, ³J_{CF}=8.0 Hz), 116.3 (d, ²J_{CF}=23.4 Hz), 118.2, 153.3 (d, ⁴J_{CF}=2.2 Hz), 158.0 (d, ¹J_{CF}=240 Hz), 168.2, 172.3, 178.9.

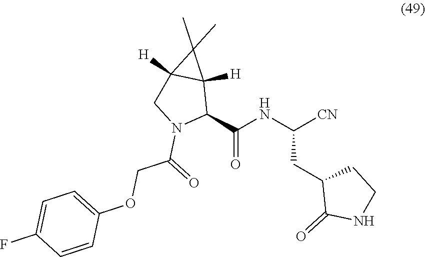 [SMILES: CC1(C)[C@@H]2[C@@H](C(=O)N[C@H](C#N)C[C@@H]3CCNC3=O)N(C(=O)COc3ccc(F)cc3)C[C@@H]21]

(1R,2S,5S)—N—((S)-1-cyano-2-((S)-2-oxopyrrolidin-3-yl)ethyl)-3-(2-(4-fluorophenoxy)acetyl)-6,6-dimethyl-3-azabicyclo[3.1.0]hexane-2-carboxamide (Cmpd No. 49). Prepared by Method A. Purified by silica gel chromatography using chloroform/methanol (0-2% methanol step gradient) to afford 13.2 mg (54%) of 49 after overnight drying at 40° C. under house vacuum: ¹HNMR (CDCl₃), rotamer ratio 79:21, line list given for the dominant rotamer, d 0.90 (s, 3H), 1.06 (s, 3H), 1.56-1.61 (2m, 2H), 1.74-1.87 (m, 1H), 1.91-1.96 (m, 1H), 2.26-2.34 (m, 2H), 2.49-2.56 (m, 1H), 3.20-3.32 (m, 2H), 3.56 (d, 1H, J=10.3 Hz), 3.90 (dd, 1H, J=9.5 and 5.6 Hz), 4.33 (s, 1H), 4.55 (s, 2H), 4.87 (ddd, 1H, J=12.7, 10.2, and 6.3 Hz), 6.19 (s, 1H, NH), 6.82-6.86 (m, 2H), 6.92-6.98 (m, 2H), 8.36 (d, 1H, J=6.7 Hz, NH); ¹³CNMR (CDCl₃), line list given for the dominant rotamer, d 12.7, 19.3, 26.2, 27.8, 28.3, 30.0, 33.6, 37.8, 39.4, 40.5, 46.6, 60.9, 67.7, 115.8 (d, ³J_{CF}=8.1 Hz), 116.0 (d, ²J_{CF}=23.3 Hz), 118.4, 154.0, (d, ⁴J_{CF}=2.1 Hz), 157.7 (d, ¹J_{CF}=239 Hz), 166.9, 171.2, 179.3.

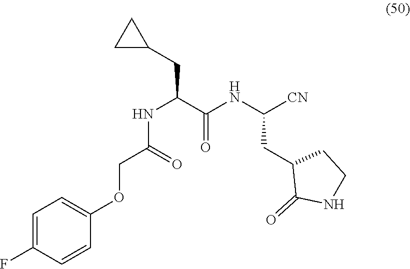 [SMILES: N#C[C@H](C[C@@H]1CCNC1=O)NC(=O)[C@H](CC1CC1)NC(=O)COc1ccc(F)cc1]

(S)—N—((S)-1-cyano-2-((S)-2-oxopyrrolidin-3-yl)ethyl)-3-cyclopropyl-2-(2-(4-fluorophenoxy)acetamido)-propenamide (Cmpd No. 50) Prepared by Method A. A vessel containing Compound 24 (17.1 mg, 39.4 mol) was purged with argon gas prior to capping with a rubber septum and solubilizing the starting material with 1 mL of dichloromethane distilled over calcium hydride. The solution was stirred while adding Burgess reagent (28.3 mg, 118.8 mol, 3 eq) to the vessel in two aliquots over the course of an hour. The reaction mixture was allowed to stir for two days, with the resulting reaction mixture transferred to a separatory funnel with roughly 17 mL of chloroform. The mixture was then washed with 3 mL of 1N hydrochloric acid followed by 3 mL of saturated sodium carbonate and 3 mL of deionized water, with the organic layer filtered using phase separating paper and concentrated under house vacuum, affording 15.8 mg of crude product. This product was then solubilized in 400 mL of chloroform and dispensed onto a 500-mg HyperSep SI cartridge of silica gel. The material was then eluded with chloroform/methanol mobile phases (0-4% methanol) in step gradient manner to afford 11.1 mg (68%) of 50 after overnight drying at 40° C. under house vacuum: ¹HNMR (methanol-d₄), d 0.06-0.19 (m, 2H), 0.44-0.52 (m, 2H), 0.66-0.76 (m, 1H), 1.53-1.61 (m, 1H), 1.74-1.84 (m, 2H), 1.85-1.94 (m, 1H), 2.21-2.33 (m, 2H), 2.51-2.61 (m, 1H), 3.18-3.28 (m, 2H), 4.41 (t, 1H, J=7.1 Hz), 4.49-4.60 (m, 2H), 5.03 (dd, 1H, J=10.2 and 5.9 Hz), 6.94-7.05 (m, 4H); ¹³CNMR (methanol-d₄), d 1.3, 1.4, 4.8, 25.0, 31.5, 34.1, 35.5, 36.2, 37.8, 51.5, 65.0, 113.3 (d, ²J_{CF}=23.5 Hz), 113.6 (d, ³J_{CF}=8.1 Hz), 116.1, 151.8 (d, ⁴J_{CF}=2.2 Hz), 155.6 (d, ¹J_{CF}=238 Hz), 167.4, 170.3, 177.4.

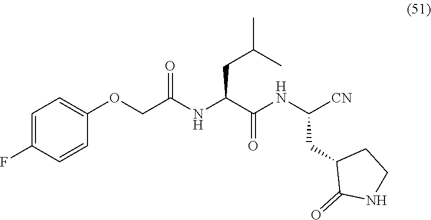 [SMILES: CC(C)C[C@H](NC(=O)COc1ccc(F)cc1)C(=O)N[C@H](C#N)C[C@@H]1CCNC1=O]

(S)—N—((S)-1-cyano-2-((S)-2-oxopyrrolidin-3-yl)ethyl)-2-(2-(4-fluorophenoxy)acetamido)-4-methylpentanamide (Cmpd No. 51). Prepared by Method A. The crude sample was loaded onto a 500-mg HyperSep SI silica gel cartridge as a solution in 2-3 mL of chloroform. The column was eluted using chloroform and chloroform/methanol phases (0-2% methanol) to give 10.9 mg (64%) of 51. ¹HNMR (CDCl₃) δ 0.93 (d, 3H, J=6.3 Hz), 0.94 (d, 3H, J=6.3 Hz), 1.52-1.65 (m, 2H), 1.65-1.74 (m, 1H), 1.75-1.89 (m, 1H), 1.92-2.02 (m, 1H), 2.32-2.46 (m, 3H), 3.29-3.42 (m, 2H), 4.45 (s, 2H), 4.66-4.77 (2m, 2H), 6.49 (s, 1H), 6.84-6.90 (m, 2H), 6.96-7.03 (m, 2H), 7.08 (d, 1H, J=8.7 Hz), 8.6 (d, 1H, J=6.4 Hz); ¹³CNMR (CDCl₃) d 21.0, 21.8, 23.7, 27.2, 32.5, 37.0, 38.3, 39.5, 40.8, 49.9, 66.9, 114.9 (d, ³J_{CF}=8.1 Hz), 115.2 (d, ²J_{CF}=23.2 Hz), 117.2, 152.2 (d, ⁴J_{CF}=2.1 Hz), 157.0 (d, ¹J_{CF}=240 Hz), 167.2, 171.2, 177.9.

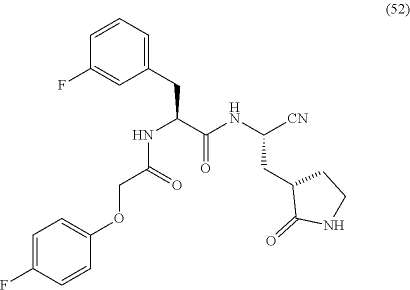 [SMILES: N#C[C@H](C[C@@H]1CCNC1=O)NC(=O)[C@H](Cc1cccc(F)c1)NC(=O)COc1ccc(F)cc1]

(S)—N—((S)-1-cyano-2-((S)-2-oxopyrrolidin-3-yl)ethyl)-2-(2-(4-fluorophenoxy)acetamido)-3-(3-fluoro-phenyl)propenamide (Cmpd No. 52). Prepared by Method A. Purified by silica gel chromatography using chloroform/methanol (0-2% methanol step gradient) to afford 9.9 mg (39%) of 52 after overnight drying at 40° C. under house vacuum: ¹HNMR (methanol-d₄), d 1.76-1.87 (m, 1H), 1.86-1.93 (m, 1H), 2.21-2.33 (2m, 2H), 2.51 (ddd, 1H, J=14.7, 9.2, and 5.6 Hz), 3.06 (dd, 1H, J=13.7 and 8.4 Hz), 3.20 (dd, 1H, J=13.7 and 6.3 Hz), 3.24-3.33 (m, 2H), 4.47 (d, 1H, J=15.0 Hz), 4.54 (d, 1H, J=15.0 Hz), 4.66 (dd, 1H, J=8.2 and 6.4 Hz), 5.01 (dd, 1H, J=10.0 and 6.0 Hz), 6.89-6.93 (m, 2H), 6.95-7.06 (m, 5H), 7.27-7.32 (m, 1H); ¹³CNMR (methanol-d₄) δ 27.2, 33.6, 36.7, 37.7, 38.5, 40.0, 54.1, 67.3, 113.4 (d, ²J_{CF}=21.2 Hz), 115.5 (d, ²J_{CF}=23.6 Hz), 115.7 (d, ²J_{CF}=21.4 Hz), 115.8 (d, ³J_{CF}=8.2 Hz), 118.1, 124.9 (d, ⁴J_{CF}=2.7 Hz), 129.9 (d, ³J_{CF}=8.3 Hz), 139.2 (d, ³J_{CF}=7.4 Hz), 154.0 (d, ⁴J_{CF}=2.2 Hz), 157.8 (d, ¹J_{CF}=238 Hz), 162.9 (d, ¹J_{CF}=245 Hz), 169.7, 171.4, 179.5.

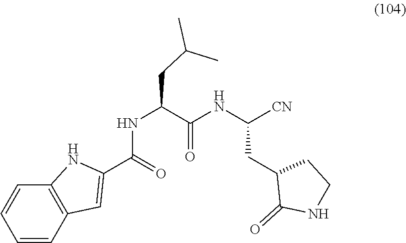 [SMILES: CC(C)C[C@H](NC(=O)c1cc2ccccc2[nH]1)C(=O)N[C@H](C#N)C[C@@H]1CCNC1=O]

N—((S)-1-(((S)-1-cyano-2-((S)-2-oxopyrrolidin-3-yl)ethyl)amino)-4-methyl-1-oxopentan-2-yl)-1H-indole-2-carboxamide (Cmpd No. 104). Prepared by Method B. Compound 104 was prepared on a 41.8 mmol scale. It was purified by silica gel chromatography using chloroform/methanol (0-2% methanol step gradient) to afford 9.8 mg (57%) of 104 after overnight drying at 40° C. under house vacuum: ¹HNMR (methanol-d₄) d 1.02 (d, 3H, J=6.4 Hz), 1.05 (d, 3H, J=6.3 Hz), 1.68-1.74 (m, 1H), 1.76-1.86 (m, 3H), 1.93 (ddd, 1H, J=13.8, 9.5, and 6.0 Hz), 2.30 (dddd, 1H, J=11.4, 9.1, 6.7, and 2.7 Hz), 2.34 (ddd, 1H, J=13.9, 10.2, and 5.3 Hz), 2.62 (ddd, 1H, J=14.9, 9.5, and 5.3 Hz), 3.24-3.32 (m, 2H), 4.61 (dd, 1H, J=9.8 and 5.4 Hz), 5.07 (dd, 1H, J=10.2 and 6.0 Hz), 7.08 (t, 1H, J=7.9 Hz), 7.21 (s, 1H), 7.23 (t, 1H, J=8.0 Hz), 7.45 (d, 1H, J=8.3 Hz), 7.63 (d, 1H, J=8.1 Hz); ¹³CNMR (methanol-d₄) δ 20.6, 22.0, 24.7, 27.1, 33.7, 37.7, 38.4, 40.0, 40.1, 52.0, 104.0, 111.6, 118.4, 119.8, 121.5, 123.8, 127.5, 130.2, 137.0, 162.7, 173.6, 179.6.

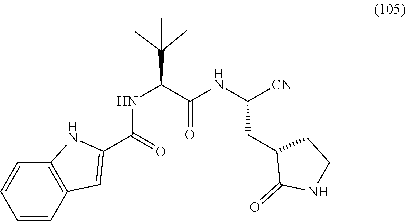 [SMILES: CC(C)(C)[C@H](NC(=O)c1cc2ccccc2[nH]1)C(=O)N[C@H](C#N)C[C@@H]1CCNC1=O]

N—((S)-1-(((S)-1-cyano-2-((S)-2-oxopyrrolidin-3-yl)ethyl)amino)-3,3-dimethyl-1-oxobutan-2-yl)-1H-indole-2-carboxamide (Cmpd No. 105). Prepared by Method B. Compound 105 was prepared on a 32.9 mmol scale. It was purified by silica gel chromatography using chloroform/methanol (0-2% methanol step gradient) to afford 9.1 mg (67%) of 105 after overnight drying at 40° C. under house vacuum: ¹HNMR (methanol-d₄) δ 1.15 (s, 9H), 1.84 (ddd, 1H, J=18.9, 12.4, and 9.0 Hz), 1.95 (ddd, 1H, J=14.0, 9.1, and 6.5 Hz), 2.29-2.35 (m, 2H), 2.61 (ddd, 1H, J=14.7, 9.0, and 5.7 Hz), 3.24-3.31 (m, 2H), 4.52 (s, 1H), 5.08 (dd, 1H, J=9.7 and 6.4 Hz), 7.09 (t, 1H, J=7.5 Hz), 7.23 (s, 1H), 7.25 (t, 1H, J=7.7 Hz), 7.45 (d, 1H, J=8.3 Hz), 7.64 (d, 1H, J=8.1 Hz); ¹³CNMR (methanol-d₄) δ 25.0, 26.4, 32.7, 33.6, 36.9, 37.5, 39.2, 60.0, 99.0, 102.0, 104.7, 118.3, 118.6, 125.1, 128.7, 138.6, 154.3, 162.3, 171.5, 179.5.

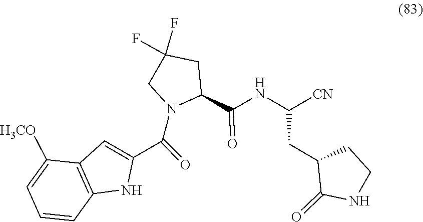

(S)—N—((S)-1-cyano-2-((S)-2-oxopyrrolidin-3-yl)ethyl)-4,4-difluoro-1-(4-methoxy-1H-indole-2-carbonyl)pyrrolidine-2-carboxamide (Cmpd No. 83). Prepared by Method A. Purification by silica gel chromatography using chloroform/methanol mobile phases (0-4% methanol) gave 4.7 mg (35%) of 83; ¹HNMR (methanol-d₄) δ 1.36-3.18 (several broad multiplets, 8H), 3.23-3.35 (m, 2H), 3.94 (br s, 3H), 4.03-4.58 (br s, 2H), 4.97-5.58 (2 br s, 1H), 6.53 (d, 1H, J=7.4 Hz), 7.06 (d, 1H, J=8.3 Hz), 7.07 (br s, 1H), 7.20 (t, 1H, J=8.0 Hz); LRMS (ESI+) m/z: [M+H]⁺ Calcd for C₂₂H₂₃F₂N₅O₄ 460.2; Found 460.2.

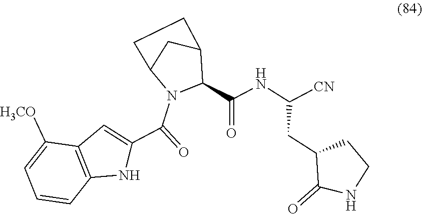

(3S)—N—((S)-1-cyano-2-((S)-2-oxopyrrolidin-3-yl)ethyl)-2-(4-methoxy-1H-indole-2-carbonyl)-2-azabicyclo[2.2.1]heptane-3-carboxamide (Cmpd No. 84). Prepared by Method A. Purified by silica gel chromatography using chloroform/methanol (0-2% methanol step gradient) to afford 7.5 mg (68%) of 84 after overnight drying at 40° C. under house vacuum: ¹HNMR (methanol-d₄), rotamer ratio 51:49, line list given for the dominant rotamer d 1.29-2.20 (several multiplets, 8H), 2.15-2.42 (several multiplets, 3H), 2.56-2.89 (m, 2H), 3.31-3.35 (m, 2H), 3.93 (s, 3H), 4.08 (s, 1H), 4.96 (br s, 1H), 5.04 (dd, 1H, J=10.1 and 5.8 Hz), 6.52 (d, 1H, J=7.6 Hz), 7.01 (d, 1H, J=8.3 Hz), 7.04 (s, 1H), 7.13 (t, 1H, J=8.0 Hz); ¹³CNMR (methanol-d₄), line list given for the dominant rotamer d 27.2, 27.3, 30.8, 33.9, 34.0, 36.9, 37.8, 40.1, 41.3, 54.2, 59.3, 66.7, 98.8, 102.4, 104.7, 118.4, 118.7, 125.1, 128.3, 137.7, 154.2, 161.5, 171.2, 179.7.

### Biological Assays and Results

The following examples illustrate but do not limit the disclosure. One of skill in the art will recognize that the following assays and methods may be modified by choice of suitable materials and methods. A coronavirus was used where a virus is introduced for an infection.

### Example A: Plaque Assay

In each of six wells about 8×10⁵ Vero-Furin cells were plated and grown in DMEM+10% FBS overnight. The next day, the cells were washed and the previous days media was replaced with DMEM+2.5% FBS and virus. The cells and viral solution were allowed to incubate for at least about two hours. The solution was then removed from the cells and replaced with a 1:1 mix of 2% agarose to 2×DMEM+5% FBS. The six well plate was then allowed incubate for three days in a tissue culture (TC) incubator. After three days, the cells were fixed in 4% formaldehyde and stained with 0.5% crystal violet solution. The resulting plaques were then counted and recorded to get an understanding of the infection rate without an inhibitor present.

### Example B: Inhibition of Viral Replication

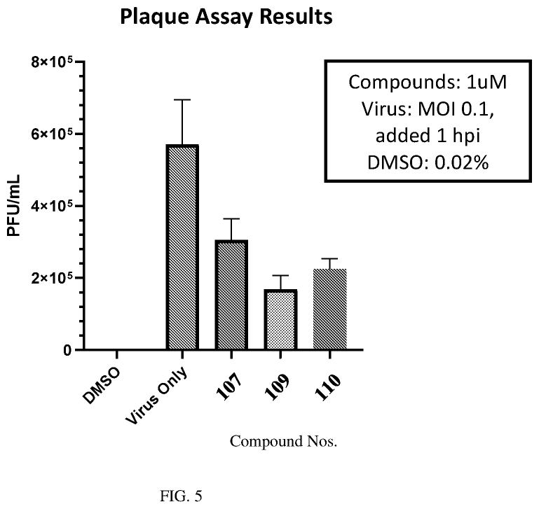

Turning to FIGS. 5, 6, 7, and 8 a tissue culture plate containing a number of wells was prepared with DMEM+10% FBS and 4×10⁴ cells per well. The cells were allowed to grow overnight. The next day, 0.1 moi of virus mixed with DMEM+2.5% FBS was added for a total of 8×10³ virions per well. The cells and viral solution were allowed incubate for at least about one hour. After at least about one hour, the viral solution was removed and a solution of DMEM+2.5% FBS and an inhibitor was added to the cells and incubated for about one day. The concentration of the inhibitor was chosen from 1 micromolar to 100 micromolar. After about one day, the cells were washed and prepared according to Example A. The plaques were then counted and recorded. Negative and positive controls were used. Negative control was DMSO instead of an inhibitor. Positive control was Nirmatrelvir.

### Example C: Direct Inhibition of Plaques Assay

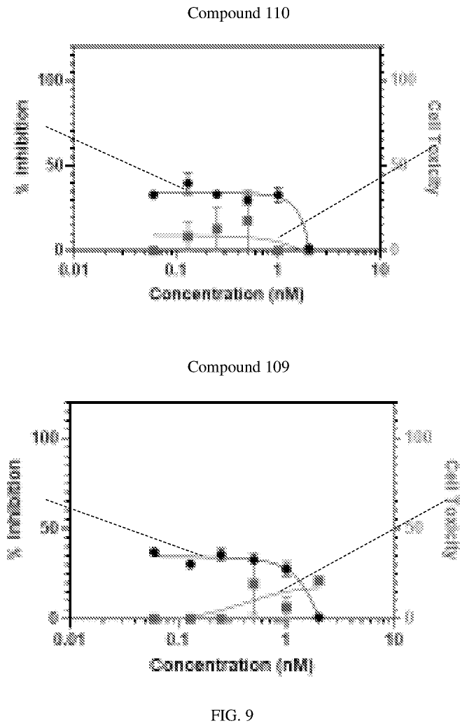

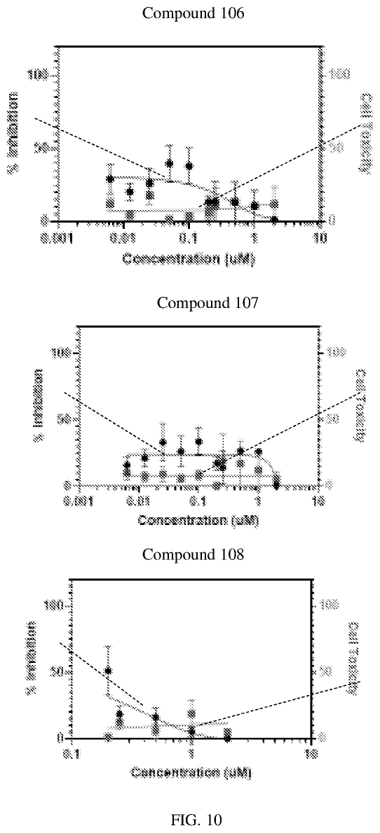

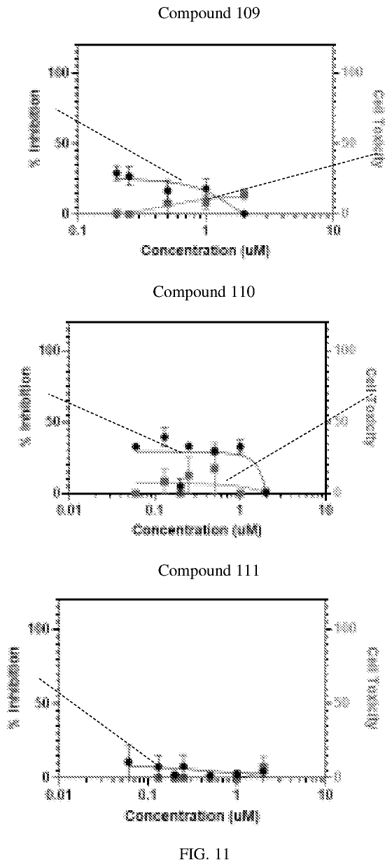

Turning to FIGS. 9, 10, and 11, a 12-well tissue culture plate was prepared by plating 4×10⁵ Vero-Furin cells per well in DMEM+10% FBS. The cells were allowed to grow overnight. The next day, a solution containing DMEM+2.5% FBS and 100 virions per well was added to each well. The virus solution was allowed to incubate with the cells of about two hours. Next, the virus solution was remove from each well, and a solution of DMEM and inhibitor was added to each. The cells were allowed to incubate for 3 days. After three days, the plaques were identified and counted as described in Example A.

### Example D: Mpro Inhibition Screening Assay

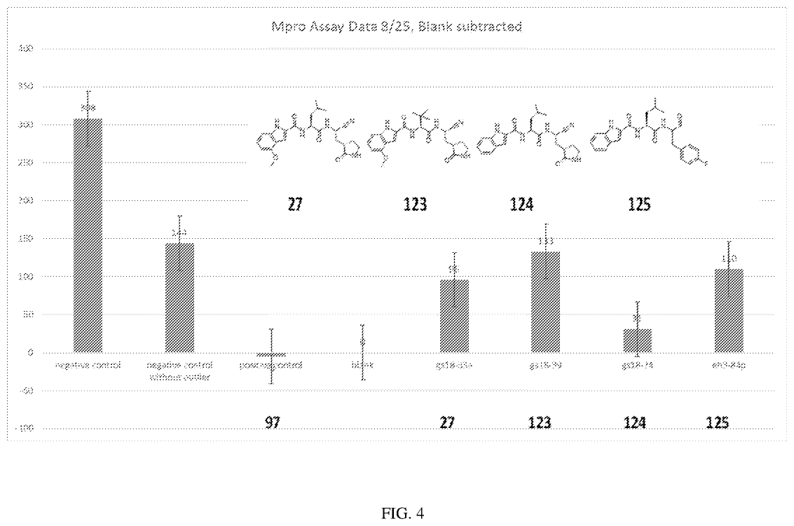

In order to assess whether the disclosed inhibitors had specific activity against the SARS-CoV conserved protein main protease (Mpro), the described inhibitors were screened as follows. Using a 3CL Fluorogenic Assay Kit sold by BPS BIOSCIENCE, one of skill in the art can determine an inhibitors ability to disrupt the protease activity of Mpro. Specifically, 3CL Protease Substrate is an internally quenched 14-mer fluorogenic (FRET) peptide (DABCYL-KTSAVLQSGFRKME-EDANS) (SEQ. ID. NO. 1). When the donor (EDANS) and acceptor (DABCYL) fluorophores are in close proximity, the energy emitted from EDANS is quenched by DABCYL (intact substrate). Upon proteolysis by Mpro, the peptide substrate is cleaved between glutamine and serine by the protease to generate the highly fluorescent peptide fragment (SGFRKME-EDANS) (SEQ. ID. NO. 2). Results are shown in FIG. 4 and Table 1, below.

The following materials were used for this assay. 1 Mpro enzyme tube containing 6 μL (40 μM) stored in 50% ethylene glycol, 1 Assay buffer tube containing 2.8 mL, Nirmatrelvir Positive Control Inhibitor (100 μM), 1 tube of Mpro Substrate containing 9.0 μL (5 mM), 1 tube containing DTT (0.5M), Test inhibitors (100 μM), 1 Black clear bottom rounded wells, a low binding 384 well plate, pipettes and tips in the following ranges P1000, P200, and P20, a Multichannel pipette 5-50 μL, 6 small centrifuge tubes, 1 clear 96 well plate, and a fluorescence spectrometer capable of measuring emission/excitation @340/490 nm.

SARS-CoV-2 main protease (3CL) enzyme (6×-His tag)=34.8 kDa

Substrate: Dabcyl-KTSAVLQSGFRKME-Edans (SEQ. ID. NO. 1)

Purchased from BPS Bio:

$$K m = 17 µM$$

Molecular Formula: C₉₅H₁₄₁N₂₅O₂₄S₂
Molecular Mass: 2081.45 g

Remove the Assay buffer tube from the ice and allow it to thaw at room temperature or hold the tube in a clasped hand to speed up the thawing. Allow the Assay buffer to completely thaw before using.

Diluent (5% DMSO): Remove 228 μL of Assay buffer from the Assay buffer tube and add to a small centrifuge tube. Add 12 μL of DMSO. Pipette in and out to mix. Label the tube “Diluent”.

Assay Buffer (1 mM DTT): The Assay buffer tube will now contain 2686 μL after making the diluent solution.
Prepare ≈2572 μL of Assay buffer(1 mM DTT). Add 5.3 μL of 0.5M DTT. Label the tube “Stock Buffer+DTT”. Invert the tube multiple times to mix. Remove 180 μL buffer from “Stock buffer+DTT” tube and add to a small centrifuge tube and label “Assay buffer +DTT”. Unused stock Assay buffer+DTT should be discarded is not used that day.

Alternatively, add 2495p L of buffer to a centrifuge tube, then add 5p L of 0.5M DTT. Label the tube “Stock Buffer+DTT”. Invert the tube multiple times to mix. Remove 180 μL buffer from “Stock buffer+DTT” tube and add to a small centrifuge tube and label “Assay buffer+DTT”.

Nirmatrelvir (Positive Control Inhibitor): Prepare a 5 μM solution of the control inhibitor. Provided with 100 μM solution, add 5 μL (100 μM) of the stock Nirmatrelvir to a labeled microfuge tube. Then add 95 μL of buffer from “Stock Buffer+DTT” to the tube. Pipette to mix.

Test Inhibitors: Prepare 100 μL (5 μM) of each of the 4 test Inhibitors. Add 5 μL (100 μM) of the stock test inhibitor to a labeled microfuge tube. Then add 95 μL of buffer from “Stock Buffer+DTT” to each tube. Pipette to mix.

Mpro enzyme: Prepare 1100 μL (67 nm) of Mpro enzyme in “Stock Assay buffer+DTT”. Add 1.85 μL of the stock enzyme(40 um) to a centrifuge tube. Label the tube “Enzyme”. Transfer 1098 μL of buffer from “Stock Buffer+DTT” to the centrifuge tube.

Substrate: Allow the Assay buffer+DTT to reach room temperature before adding to the substrate. Prepare 360 μL (125 μM) of substrate solution. Label microfuge tube “Substrate.” Add 9.0 μL (5 mM) of substrate to a microfuge tube. Then add 351 μL of room temperature “Stock Buffer+DTT”. Pipette to mix.

Plate prep: Add all the corresponding components to the appropriate wells of the plate (excluding the substrate). Once the plate is loaded, using a multichannel pipette, fill a second plate. Lightly tap the sides to mix or place on a shaker at low speed. Allow the plates to sit for about 30 minutes, incubation period is to allow the potential inhibitors to interact with the enzyme before the substrate is added. After about 30 minutes, add 10 μL of Substrate (125 μM) to all wells to start the reaction.

Fluorescence spectrometer settings: Excitation wavelength=340 nm, Emission wavelength=490 nm. Results are provided in Table 1.

TABLE 1

| | % Inhibition of Mpro @ | % Inhibition of Mpro @ |
|---|---|---|
| Compound No. | 1 μM (Single dose) | 100 μM (Single dose) |
| 27 | Nt | 33 |
| 123 | Nt | 7 |
| 124 | Nt | 79 |
| 97 | Nt | 97 |
| Nirmatrelvir | 99 | 96 |
| (positive control) | | |
| 125 | Nt | 24 |
| 83 | Nt | 64 |
| 84 | Nt | 54 |
| 35 | 22 | Nt |
| 46 | 21 | Nt |
| 43 | 20 | Nt |
| 51 | Nt | 0 |
| 32 | 8 | 0 |
| 47 | <1 | Nt |
| 44 | 10 | Nt |
| 128 | 71 | Nt |
| 53 | 0 | 35 |

Nt = not tested

1. A compound having the general structure of: 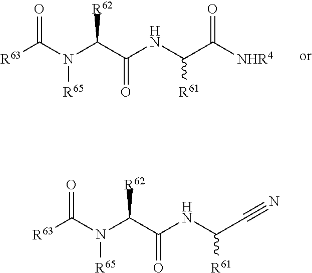
wherein
R⁶¹ is C₁-C₆ alkyl, (C₁-C₄ alkyl)R²⁵ 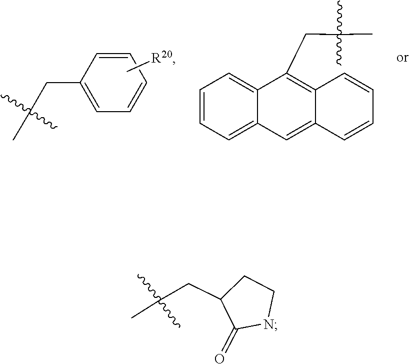
R⁶² is selected from the group consisting of C₁-C₅ alkyl, 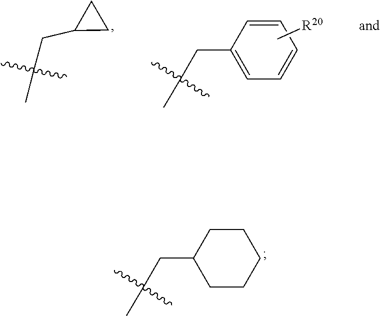
R⁶³ is selected from the group consisting of 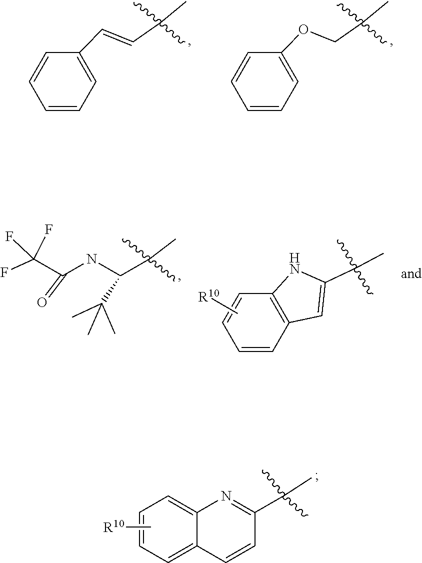
R⁴ is selected from the group consisting of H, C₁-C₄ alkyl, (C₀-C₂ alkyl)cyclopropyl, and c-propylmethyl;
R⁶⁵ is H, or R⁶² and R⁶⁵ together with the atoms to which they are attached form an optionally substituted 5 or 6 membered heterocyclic ring or a 7 membered bicyclic ring, wherein the substituents of the optionally substituted 5 or 6 membered heterocyclic ring are halo, optionally fluorine, optionally wherein the 7 membered bicyclic ring is norbornane;
R¹⁰ is H, halo or —O(C₁-C₄ alkyl);
R²⁰ is halo, optionally F or H; and
R²⁵ is halo, CN, CONHR, NHR, COOR, phenyl sulfone, or fluorophenyl, wherein R is H or C₁-C₄ alkyl.

2. The compound of claim 1 having the general structure of 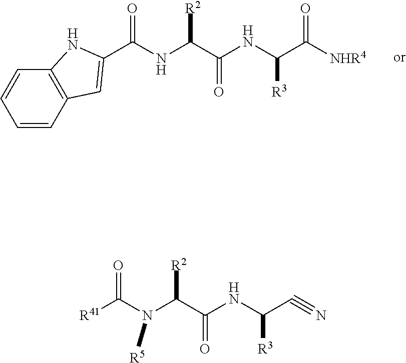
wherein
R⁴¹ is selected from the group consisting of 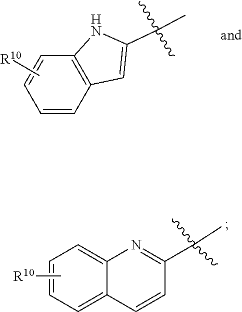
R² is selected from the group consisting of C₁-C₅ alkyl 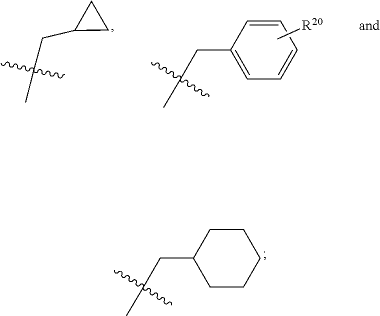
R³ is C₁-C₆ alkyl, (C₁-C₄ alkyl)R²⁵ 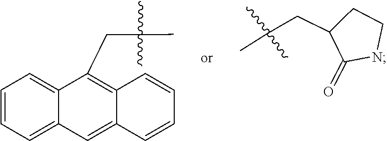
R⁴ is selected from the group consisting of H, C₁-C₄ alkyl, (C₀-C₂ alkyl)cyclopropyl, and c-propylmethyl;
R⁵ is H, or R² and R⁵ together with the atoms to which they are attached form an optionally substituted 5 or 6 membered heterocyclic ring or a 7 membered bicyclic ring, wherein the substituents of the optionally substituted 5 or 6 membered heterocyclic ring are halo, optionally fluorine, optionally wherein the 7 membered bicyclic ring is norbornane;
R¹⁰ is H, halo or —O(C₁-C₄ alkyl);
R²⁰ is halo, optionally F, or H; and
R²⁵ is halo, CN, CONHR, NHR, COOR, phenyl sulfone, or fluorophenyl, wherein R is H or C₁-C₄ alkyl.

3. The compound of claim 2 having the general structure of 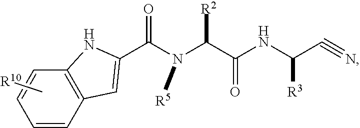
wherein
R¹⁰ is H or methoxy;
R² is selected from the group consisting of C₁-C₅ alkyl, 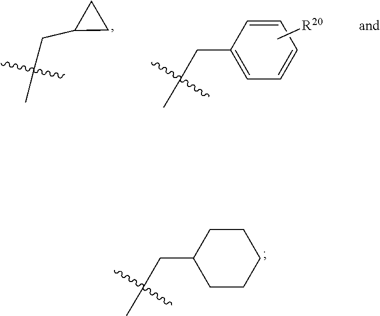
R³ is C₁-C₆ alkyl, (C₁-C₄ alkyl)R²⁵ 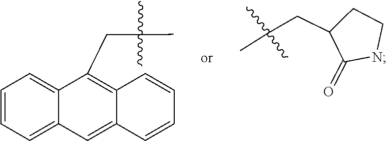
R⁵ is H, or R² and R⁵ together with the atoms to which they are attached form an optionally substituted 5 or 6 membered heterocyclic ring or a 7 membered bicyclic ring, wherein the substituents of the optionally substituted 5 or 6 membered heterocyclic ring are halo, optionally fluorine, optionally wherein the 7 membered bicyclic ring is norbornane; and
R²⁵ is halo, CN, CONHR, NHR, COOR, phenyl sulfone, or fluorophenyl, wherein R is H or C₁-C₄ alkyl.

4. A compound having the general structure of 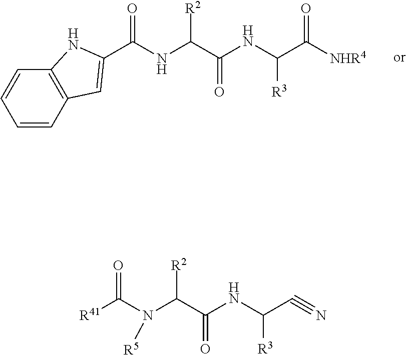
wherein
R⁴¹ is selected from the group consisting of 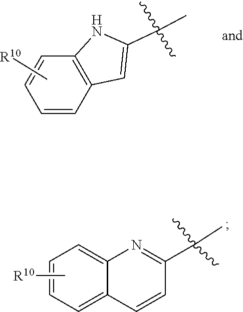
R² is selected from the group consisting of C₁-C₅ alkyl, 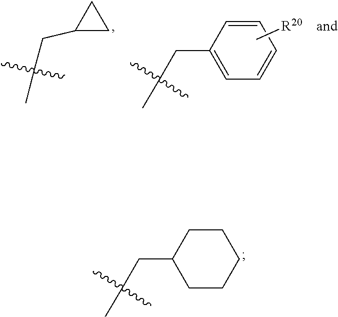
R³ is C₁-C₆ alkyl, (C₁-C₄ alkyl)R²⁵, 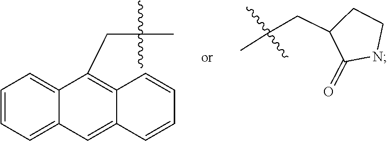
R⁴ is selected from the group consisting of H, C₁-C₄ alkyl, (C₀-C₂ alkyl)cyclopropyl, and c-propylmethyl;
R⁵ is H, or R² and R⁵ together with the atoms to which they are attached form an optionally substituted 5 or 6 membered heterocyclic ring or a 7 membered bicyclic ring, wherein the substituents of the optionally substituted 5 or 6 membered heterocyclic ring are halo, optionally fluorine, optionally wherein the 7 membered bicyclic ring is norbornane;
R¹⁰ is H, halo or —O(C₁-C₄ alkyl);
R²⁰ is halo, optionally F, or H; and
R²⁵ is halo, CN, CONHR, NHR, COOR, phenyl sulfone, or fluorophenyl, wherein R is H or C₁-C₄ alkyl.

5. The compound of claim 4 having the general structure of 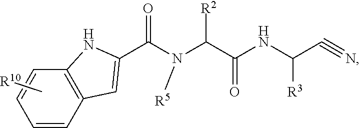
wherein
R¹⁰ is methoxy.

6. The compound of claim 1 having the general structure of 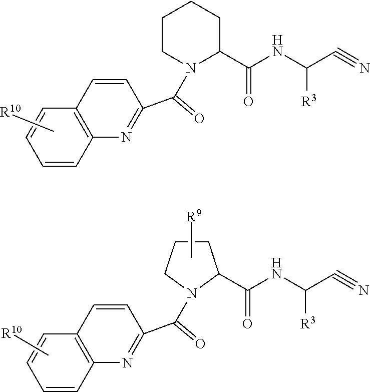
wherein R⁹ is H or F.

7. A compound in accordance with claim 1, wherein
R² is isopropyl or

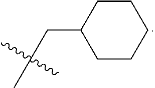 [SMILES: *CC1CCCCC1]

8. A compound in accordance with claim 1 wherein
R³ is

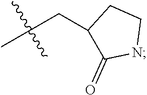

9. A compound in accordance with claim 1 wherein the compound has the structure of any of the compounds Compound No. 1-128 of the Representative Embodiments.

10. A compound having the general structure of: 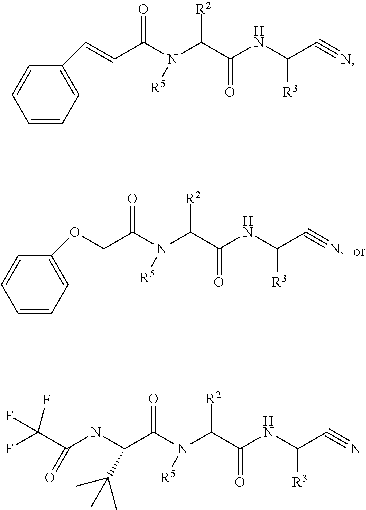
wherein
R² is selected from the group consisting of C₁-C₅ alkyl;
R³ is 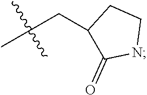
R⁵ is H, or R² and R⁵ together with the atoms to which they are attached form an optionally substituted 5 or 6 membered heterocyclic ring or a 7 membered bicyclic ring, wherein the substituents of the optionally substituted 5 or 6 membered heterocyclic ring are halo, optionally fluorine, optionally wherein the 7 membered bicyclic ring is norbornane.

11. The compound of claim 10 wherein
R² is isopropyl or R² and R⁵ together with the atoms to which they are attached form 5 or 6 membered heterocyclic ring that is optionally substituted with 1 to two fluorine atoms.

12. The compound of claim 4 having the general structure of: 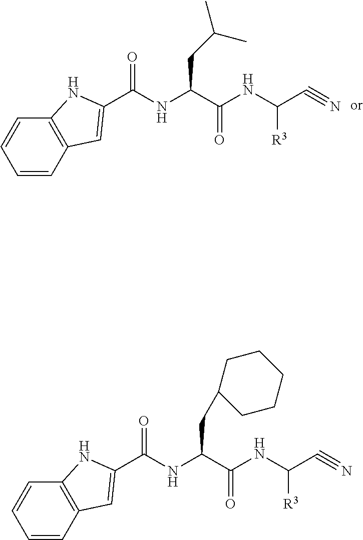
wherein
R³ is C₁-C₆ alkyl, (C₁-C₅ alkyl)R²⁵ 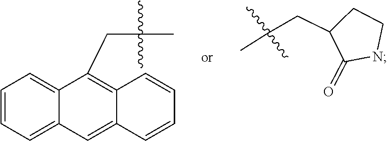
R²⁵ is halo, CN, CONHR, NHR, COOR, phenyl sulfone, or fluorophenyl, wherein R is H or C₁-C₄ alkyl.

13. An M^{pro} inhibitor of claim 1, wherein the compound has the structure of 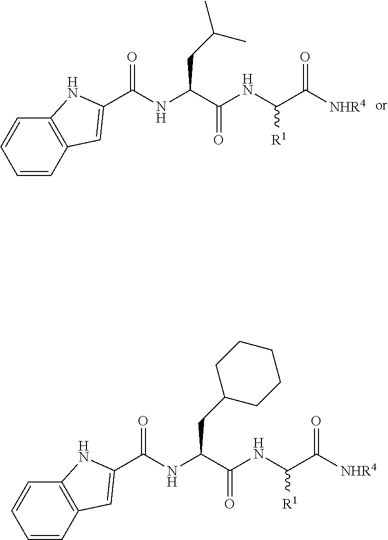
wherein R¹ is C₁-C₆ alkyl, (C₁-C₄ alkyl)R²⁵ 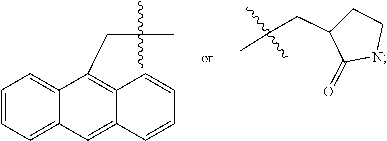
R⁴ is selected from the group consisting of H, C₁-C₄ alkyl; and
R²⁵ is halo, CN, CONHR, NHR. COOR, phenyl sulfone, or fluorophenyl, wherein R is H or C₁-C₄ alkyl.

14. A compound in accordance with claim 13 wherein R² is isopropyl.

15. A compound in accordance with claim 13 wherein
R³ is

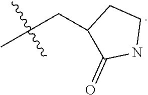 [SMILES: *CC1CCNC1=O]

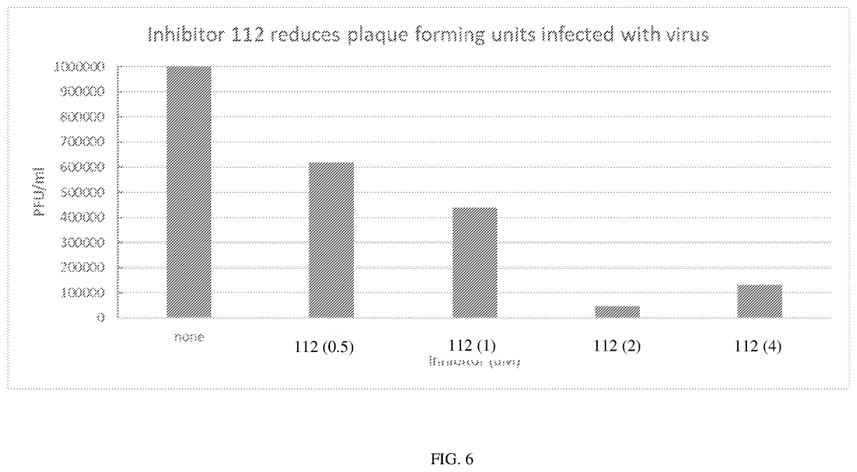

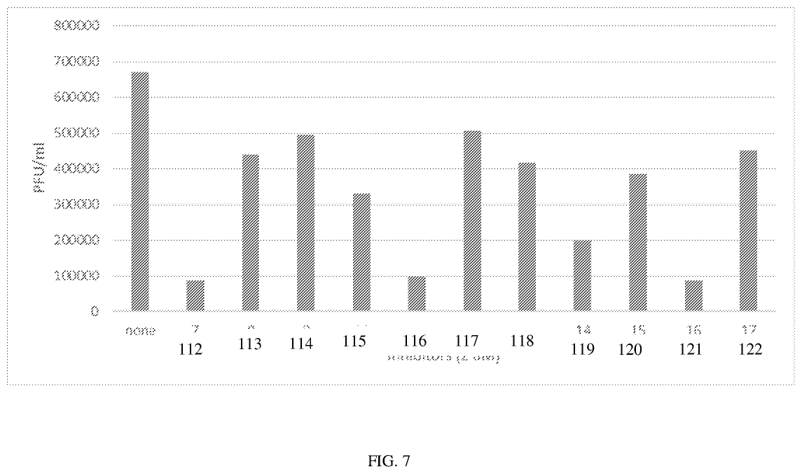

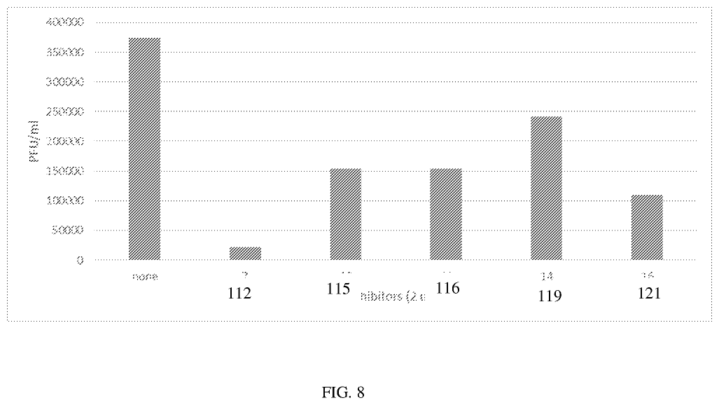

16. A compound in accordance with claim 13 wherein the compound has the structure of any of the compounds of FIG. 5, FIG. 6, FIG. 7, or FIG. 8.

17. A method of inhibiting the replication of severe acute respiratory syndrome coronavirus 2 (SARS-CoV-2), said method comprising contacting the virus with a composition comprising a compound of claim 1.
